(19) United States
(12) Patent Application Publication
(10) Pub. No.: US 2019/0231932 A1
(43) Pub. Date: Aug. 1, 2019

(54) COMPLEX LIVING INTERFACE-COORDINATED SELF-ASSEMBLING MATERIALS (CLICSAM)

(71) Applicant: PolarityTE, Inc., Salt Lake City, UT (US)

(72) Inventors: Denver Lough, Salt Lake City, UT (US); Nikolai Sopko, Salt Lake City, UT (US); Pratima Labroo, Salt Lake City, UT (US); Nicholas Baetz, Draper, UT (US)

(21) Appl. No.: 16/260,096

(22) Filed: Jan. 28, 2019

Related U.S. Application Data
(60) Provisional application No. 62/622,489, filed on Jan. 26, 2018.

(51) Int. Cl. A61L 27/38; A61L 27/36

(52) U.S. Cl. CPC A61L 27/3821; A61L 27/3633; A61L 2430/02; A61L 27/3683; A61L 27/3834

(57) ABSTRACT

Disclosed herein is a composition comprising a stimulated heterogeneous mammalian tissue interface cell aggregate that is capable of producing functional polarized tissue when administered to a subject in need thereof.

### CROSS-REFERENCE TO RELATED APPLICATIONS

This application claims priority to U.S. provisional application No. 62/622,489 filed Jan. 26, 2018, the contents of which are incorporated by reference in their entirety herein.

### TECHNICAL FIELD

The present disclosure relates generally to a synthesized composition of interfacing, self-propagating cellular and non-cellular materials (an aggregate) which can be used to generate or regenerate functional material(s), tissue(s), tissue system(s), and/or tissue compartment(s) in an area in which this aggregate of materials is placed, made present or materialized. The present disclosure also relates generally to: 1.) a method of producing such a composition; 2.) maintenance, propagation and/or storage of such a composition 3.) use of such a composition. Such composition may be called a Complex-Living, Interface-Coordinated, Self-Assembling Material (“CLICSAM”).

More particularly, the present disclosure is in the technical field(s) of neo-generative and regenerative materials and substrates which may be utilized across a variety of related technical fields which may include but are not limited to: a) Medicine, b) Medical practice, c) Devices, d) Biologics, e) Therapeutics, f) Small molecule synthesis, g) Macromolecular synthesis, h) Cellular materials synthesis, i) Sub-cellular synthesis, j) Tissue engineering, k) Bioreactor development and/or support of bio-reactive support, l) Medical research, m) Medical and/or biomedical manufacturing, n) Veterinary practice, o) Veterinary research, p) Molecular biology applications, q) Chemistry and/or chemical manufacturing and/or chemical engineering, r) Material sciences, s) Food manufacturing and/or food production, t) Nutraceutical manufacturing, u) Supplement manufacturing, v) Cosmetic development, w) Composite life systems, x) Artificial intelligent systems, y) Agriculture, z) Space research efforts and/or exploration, aa) Defense, weapons or military application(s), bb) Transplantation, immunology, tolerance and/or immune modulation of materials.

### BACKGROUND

A variety of synthetic, inorganic, organic and composite technologies and/or systems have been developed which rely on intrinsic thermodynamics forces to cause structural memory or assembly memory to drive change in systems. This type of regain of structure or assembly in a system is because it is thermodynamically favorable for such materials to organize in such manner, not because the material recognizes, senses, calculates and self-determine the response to the environment in which the material is/was placed.

The generation, regeneration, materialization and/or propagation of functionally-polarized, hierarchically-organized materials, substrates, tissue(s) and/or tissue system(s) have remained of interest in a variety of fields. Despite much interests and significant research into the development of material compositions and/or mechanisms of creating synthetic, substitutive, or altered forms of self-propagating materials, substrates, or tissue elements, such matter has not been tangibly created, established or developed.

Conventional theory, teaching and practice have continued to iterate three traditionally reductionist approaches to those efforts in pursuance to engineering, generation, regeneration, development and/or materialization of dynamic living tissue systems. These three traditional iterative approaches have commonly been referred to in published literature as a tissue engineering triad, with each point of the triad being associated with the basis of the engineered material(s). These approaches can be summarized as: 1.) a cell-based approach; 2.) a molecular-based approach; and 3.) a scaffold and/or matrix-based approach.

In theory, teaching and practice, these approaches have classically been promoted and utilized in singularity, derivatized singular systems, iterative combination(s) and/or combinatorial associations.

The cell-based approach commonly focuses on the isolation, cultivation, development or directive action development of a cellular entity to regenerate cell(s), tissue(s) related product(s) and/or to promote, drive, direct or command cells, cellular processes and/or tissues toward a biological pathway or functional outcome.

The molecular-based approach commonly focuses on the delivery of an agent (e.g., factor(s), drug(s), a gene(s) directive agent(s), particle(s)) to promote, drive, direct or command cells, cellular processes and/or tissues toward a biological pathway or functional outcome.

The scaffold or matrix based approach focuses on the use of some form of a supportive structure (e.g., a scaffold, matrix, fiber, particle), vectoral and/or carrier into a system which promotes either: 1.) cellular migration, differentiation, and/or propagation from surrounding native tissues and/or 2.) acts as a carrier of cellular entities and/or agent(s) into the tissue system.

The three traditional approaches are reductionist and incomplete as such approaches are assembled in a manner which seeks the pursuit of developing, synthesizing, and/or engineering resultant complex systems from finite and restricted cells, agents and structures which are synthetically limited and lack dynamic capabilities. As such, these limited approaches are incongruent with life in that they attempt to act as a finitely complete answer to a complex and evolving system (a tissue and/or living material substrate void requiring substantive and functional generation, regeneration and/or self-propagation).

Moreover, subsequent to the delivery of such conventional yet limited approach(es), the receiving complex, evolving, reactive and dynamic system which exists within an organism, system, or environment reacts acutely and/or chronically reacts or responds to or toward the foreign, synthetic, different, and/or altered material comprising the delivered cell, agent and/or triad-derived structure. These reactions in turn often result in drastic alterations to and/or within the delivered product as well as within the local native environment, interdependent associated system(s) and pathway(s).

The incongruent actions between the 1.) deployed traditional triad-derived incomplete approaches (cell, agent and/or structures) and 2.) the reactive complex system result in failure to deliver true generation, regeneration and/or propagation of the complete system (i.e., functionally-polarized, hierarchically-organized materials, substrates, tissue(s) and/or tissue system(s)).

Thus, there remains a need for a technology which can be utilized for the generation, regeneration, materialization and/or propagation of functionally-polarized, hierarchically-organized materials, substrates, tissue(s) and/or tissue system(s).

### SUMMARY

One aspect of the present disclosure relates to a composition comprising a stimulated heterogeneous mammalian tissue interface cell aggregate that is capable of producing functional polarized tissue when administered to a subject in need thereof.

One aspect of the present disclosure relates to a composition comprising at least a portion of a mammalian material interface. The mammalian material interface comprises core potent cellular entities and supportive entities. The composition is capable of assembling functional material.

Another aspect of the present disclosure relates to a method of producing a composition. The method comprises isolating at least a portion of a mammalian material interface comprising core potent cellular entities and supportive entities. The method further comprises developing a reactive and stimulated interface to provide the composition. The composition is capable of assembling functional material.

### BRIEF DESCRIPTION OF THE DRAWINGS

The patent or application file contains at least one drawing executed in color. Copies a this patent or patent application publication with color drawing(s) will be provided by the Office upon request and payment of the necessary fee.

FIGS. 1a-1e illustrate an osseous-derived composition in a cranial defect model system.

FIGS. 2a and 2b illustrate a cutaneous-derived composition in a cutaneous model system.

FIG. 3 shows a heat map displaying fold change in gene expression of angiogenesis factors for an osseous-derived composition (e.g., AHBC) treated group compared to native bone in the rabbit long bone study of Example 5.

FIG. 4 shows a heat map displaying fold change in gene expression of osteogenesis genes for an osseous-derived composition (e.g., AHBC) treated group compared to native bone in the rabbit long bone study.

FIG. 5 shows a heat map displaying fold change in gene expression of wound healing genes for an osseous-derived composition (e.g., AHBC) treated group compared to native bone in the rabbit long bone study.

FIG. 6 shows DSLT images of native bone, defect creation, treatment, and ex vivo endpoint images at post-operative week (POW) 12 for an osseous-derived composition (e.g., AHBC) treated group and the untreated group in the rabbit long bone study.

FIG. 7 shows 3D and 2D Vimago serial CT images acquired for an osseous-derived composition (e.g., AHBC) treated group and the untreated group in the rabbit long bone study post operatively and every 2 weeks for 12 weeks. Each group is represented by one animal.

FIG. 8 shows 3D and 2D microCT images acquired for an osseous-derived composition (e g., AHBC) treated group and the untreated group in the rabbit long bone study ex vivo at post-operative week (POW) 12. Each group is represented by one animal.

FIG. 9 shows light images taken using the Leica M205 FA of both sides A and B of two samples from an osseous-derived composition (e.g., AHBC) treated group and the untreated group in the rabbit long bone study. Before the images of the AHBC treated group were taken the radius was removed from the ulna to give a clearer representation of the regrowth region. The untreated group has an attached radius due to lack of regrowth in the defect area.

FIG. 10 shows, for the rabbit long bone study, an untreated sample in Row 1 with the radius still intact due to lack of regrowth and an osseous-derived composition (e.g., AHBC) treated sample in Row 2. A and B are photographs of opposite sides of the same sample. C and D are scanning electron microscopy (SEM) micrographs of opposite sides of the same sample. E is a second harmonic multiphoton (MP) image.

FIG. 11 shows average surface point scans from native bone, untreated defects, and an osseous-derived composition (e.g., AHBC) treated group in the rabbit long bone study.

FIG. 12 shows surface line scans from native bone, untreated defects, and an osseous-derived composition (e.g., AHBC) treated group in the rabbit long bone study. Red represents a high Raman intensity while blue represents a low intensity. Line scan intensity blended view (left) and line scan z-view (right).

FIG. 13 shows surface area scans from native bone, untreated defects, and an osseous-derived composition (e.g., AHBC) treated group in the rabbit long bone study. Scans were taken at the native-defect interface. Red represents a high Raman intensity while blue represents a low intensity. Hydroxyapatite intensity is within the range 950-965 cm′.

FIG. 14 illustrates spinal fusion frequency in the rabbit spinal study of Example 6.

FIG. 15 shows bone mineral density compared to autograft treatment in the rabbit spinal study using a Dunnett's multiple comparison test.

FIG. 16 shows average cross section point scans from native bone and treated groups including an osseous-derived composition (e.g., AHBC) treated group in the rabbit spinal study.

FIG. 17 shows cross section line scans from treated groups including an osseous-derived composition (e.g., AHBC) treated group in the rabbit spinal study.

FIG. 18 shows serial Vimago CT images taken over 8 weeks including an osseous-derived composition (e.g., AHBC) treated group in the rabbit cranial study. 3D and 2D CT images are shown for one representative animal of each group.

FIG. 19 shows ex vivo microCT images captured at post-operative week (POW) 8 in the rabbit cranial study. 3D and 2D CT images are shown for one representative animal of each group.

FIG. 20 shows bone mineral density measurements for treatment groups including an osseous-derived composition (e.g., AHBC) treatment group in the rabbit cranial study at post-operative week (POW) 8. Values represent mean±standard deviation. Comparisons were made using an ordinary one-way ANOVA with Dunnett's Multiple Comparisons Test and a p<0.05 considered significant.

FIG. 21 shows trabecular bone mineral density measurements for treatment groups including an osseous-derived composition (e.g., AHBC) treatment group in the rabbit cranial study at post-operative week (POW) 8. Values represent mean±standard deviation. Comparisons were made using an ordinary one-way ANOVA with Dunnett's Multiple Comparisons Test and a p<0.05 considered significant.

FIG. 22 shows bone volume to tissue volume percentage (BV/TV) for treatment groups including an osseous-derived composition (e.g., AHBC) treatment group in the rabbit cranial study at post-operative week (POW) 8. Values represent mean±standard deviation. Comparisons were made using an ordinary one-way ANOVA with Dunnett's Multiple Comparisons Test and a p<0.05 considered significant.

FIG. 23 shows average surface point scans from native bone, untreated defects, and an osseous-derived composition (e.g., AHBC) treated group in the rabbit cranial study.

FIG. 24 shows representative surface line scans from native bone, untreated defects, and an osseous-derived composition (e.g., AHBC) treated group in the rabbit cranial study. Red represents a high Raman intensity while blue represents a low intensity. Line scan intensity blended view (left) and line scan z-view (right).

FIG. 25 shows cross-sectional area scans from native bone, untreated defects, and an osseous-derived composition (e.g., AHBC) treated group in the rabbit cranial study. Red represents a high Raman intensity while blue represents a low intensity. Collagen intensity within the range 880-840 cm⁻¹ (left), hydroxyapatite intensity within the range 950-965 cm⁻¹ (center), and reference image (right).

FIG. 26 shows representative DSLR images of native bone, defect creation, treatment and ex vivo endpoint images at post-operative week (POW) 8 for each treatment group in the rabbit cranial study.

FIG. 27 shows representative V16 compound microscope images of ex vivo crania for each treatment group in the rabbit cranial study.

FIG. 28 shows microscopy of an osseous-derived composition (e.g., AHBC) treated and untreated defects in the rabbit cranial study. Ex vivo 1 mm-thick cranial bone cross-sections were imaged using second harmonic generation microscopy (Row A), stained for nuclei (blue) with NucBlue Ready Probes (Catalog #: R37605, Molecular Probes, Eugene, Oreg., USA), Hydroxyapatite (green) with Osetoimage Mineralization Assay (Catalog #: PA-1503, Lonza, Walkersville, Md., USA), and Actin (red) with ActinRed-555 (Catalog #: R37112, Thermofisher, Eugene, Oreg., USA), imaged with confocal microscopy using a 10× objective (Row B), compound light microscopy (Row C), HDBSD detector in SEM (Row D), and C2DX detector in SEM (Row E). SHG imaging, SEM, and brightfield imaging of demineralized and glass-slide mounted 4 uM sectioned samples are shown (Row F-J).

FIG. 29 shows a heatmap produced from hierarchical clustering of osteogenesis, wound healing, and angiogenesis pathway genes (y-axis) from 4 pre- and 5 post-processed rabbit cranium samples (x-axis) representative of altered molecular pathways in osseous-derived compositions versus native osseous tissue. Dark red and yellow are associated with the highest and lowest levels of gene expression, respectively.

FIG. 30 shows a volcano plot showing gene expression differences between pre (n=4) and post (n=5) rabbit cranium for osteogenesis pathway genes.

FIG. 31 shows a volcano plot showing gene expression differences between pre (n=4) and post (n=5) rabbit cranium for wound healing pathway genes.

FIG. 32 shows a volcano plot showing gene expression differences between pre (n=4) and post (n=5) rabbit cranium for angiogenesis pathway genes.

FIG. 33 shows a heatmap representative of altered molecular pathways in a hepatic-derived composition (e.g., AHLC) versus native hepatic tissue. Dark red and yellow are associated with the highest and lowest levels of gene expression, respectively.

FIG. 34A shows a heatmap representative of targeted transcriptome analysis assessing wound healing, stem cell, and cell surface marker pathways in a cutaneous-derived composition (e.g., AHSC) versus native cutaneous tissue. Dark red and yellow are associated with the highest and lowest levels of gene expression, respectively.

FIG. 34B shows a volcano plot showing increased expression of stem cell markers in a cutaneous-derived composition (e.g., AHSC) relative to native cutaneous tissue.

FIG. 35 shows force versus displacement for an osseous-derived composition (e.g., AHBC) versus native rabbit long bone.

FIG. 36 shows hydroxyapatite chemical maps for an osseous-derived composition (e.g., AHBC) (right) versus native rabbit long bone (left).

FIG. 37 shows force versus displacement for a fat-derived composition versus native fat (human).

FIG. 38 shows force versus displacement for a muscle-derived composition versus native muscle (human).

FIG. 39 shows force versus displacement for a cartilage-derived composition versus cartilage (pig).

FIG. 40 shows force versus displacement for an osseous (femur)-derived composition versus native bone.

FIGS. 41A-C show in vivo images of wound healing captured in each treatment group of the swine compared to native swine specimen at various post-operative days (POD). FIG. 41A shows representative images of wound healing at POD 0, 19, 35, 42, and 70 for one wound size. FIG. 41B shows representative images of wound healing at POD 0, 48, 98, 146, and 196 for another wound size. FIG. 41C shows representative images of wound healing at POD 0, 34, 62, 105, 132 for yet another wound size.

FIGS. 42A-C depict relative contraction with respect to POD for each treatment group of the swine. Relative contraction was calculated. 0 is no contraction and 1 is fully contracted.

FIGS. 43A-C depict compound light microscopy, histological staining, SEM, confocal, and multiphoton imaging of cutaneous-derived composition (e.g., AHSC) treated wounds. FIGS. 43A-C show representative imaging of different wound sizes. Compound light microscopy sample overviews (column A) and cross sections (column B), Masson's Trichrome staining (column C), SEM (column D), confocal microscopy (column E), and multiphoton imaging (column F). Notably, healing was improved in cutaneous-derived composition (e.g., AHSC) treated wounds (A-8) compared with untreated wounds (A-12). Development of organized ECM was observed in histological samples, SEM, and multiphoton analysis (Columns C, D, and F). Ultrastructural elements were observed in cutaneous-derived composition (e.g., AHSC) treated wounds as shown by confocal microscopy (E).

FIGS. 44A-C show Raman surface point scan comparison of swine treatment groups against native skin for different wound sizes, respectively. The peaks at 854 cm⁻¹ (Proline), 875 cm⁻¹ (hydroxyproline), 1003 cm⁻¹ (phenylalanine), 1450 cm⁻¹ (elastin) and 1650 cm⁻¹ (keratin) are present in both treated wounds and native skin.

FIGS. 45A-C depict Raman surface point scan (left) and surface line scan (right) comparison of swine treatment groups against native skin and/or untreated wounds for different wound sizes, respectively. The peaks at 854 cm⁻¹ (Proline), 875 cm⁻¹ (hydroxyproline), 1003 cm⁻¹ (phenylalanine), 1450 cm⁻¹ (elastin) and 1650 cm⁻¹ (keratin) are present in both treated wounds and native skin.

FIG. 46A-B depict Raman cross section line scans for different wound sizes, respectively. Comprehensive Raman figures show the molecular fingerprint of swine skin. Blended view of line scans across the skin cross section are for different treatments. Chemigrams compare collagen type IV distribution across skin cross section.

FIG. 47A depicts typical force vs. displacement curves from tensile testing (Instron 3343) for swine skin wounds. The elastic modulus, i.e., measure of skin elasticity was measured using the slope of linear portion of graph.

FIG. 47B depicts results a Young's Modulus from tensile testing (Instron 3343) for swine skin wounds.

FIG. 48A depict in vivo ballistometry outcome for wounds. Initial impact called “indentation” measuring the depth of the probe impact in the skin was estimated from the damping curve.

FIG. 48B depicts in vivo elastic modulus measurement using Ultrasound Elastography (Native, Wound 1, Wound 2). In vivo elasticity measurement using Ultrasound Elastography. Real time Ultrasound Shear wave elastography was performed to evaluate the elasticity of skin. The elastic modulus calculated from the tissue compressibility represents skin stiffness. On the legend, green and red indicate soft and hard tissues, respectively. Shear wave elastography of native skin and treated wound shows homogeneous and soft zones with mean elastography values below 120 kPa.

FIG. 49 depict ex-vivo ballistometry outcome for wounds. Initial impact called “indentation” measuring the depth of the probe impact in the skin was estimated from the damping curve.

FIG. 50 shows tissue molecular analysis heatmaps displaying differential gene expression of cutaneous-derived composition (e.g., AHSC) healed wounds compared to native skin.

FIG. 51 depicts fold change of transcripts for which expression was significantly different (p<0.05) in cutaneous derived-composition (e.g., AHSC) healed wounds when compared to native skin.

FIG. 52 shows heatmaps displaying differential gene expression of cutaneous-derived composition (e.g., AHSC) treated wounds compared to untreated wounds and bar graphs describing fold change of transcripts for which expression was significantly different (p<0.05) in cutaneous-derived composition treated wounds when compared to untreated wounds. Stem cell markers are also generally upregulated. Significant differences in gene expression were observed for 12 genes: CDH1, COL7A1, COL4A3, CTNNA1, CTNND1, ITGAE, ANOS1, ITGB4, and MMP12. CDH1 is upregulated 235-fold in cutaneous-derived composition treated wounds compared to untreated wounds, and COL7A1 is upregulated 17-fold.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention. Thus, it is to be understood that other embodiments may be utilized and changes may be made without departing from the scope of the present disclosure. The following detailed description, therefore, is not to be taken in a limiting sense.

The compositions described herein have utility in a variety of technical fields, including but not limited to medicine, sciences, engineering and manufacturing. The present disclosure relates to synthesized compositions of an aggregate of dynamic, reactive three-dimensionally interfaced entities which contain both interactive, living core potent cellular entities (e.g., stem cells, progenitor cells, transit-amplifying cells) and supportive entities.

Disclosed herein is a composition of interfacing, self-propagating cellular and non-cellular materials (an aggregate) which can be used to alter the environment in which this aggregate of materials is/was placed. Such aggregate may be called a Complex-Living, Interface-Coordinated, Self-Assembling Materials (CLICSAM).

The compositions disclosed herein when developed or synthesized promote the coordinated propagation of potent cellular expansion and the organized formation of material and/or substrate for the continued propagation of the CLICSAM and those progressive intermediate derivatives which form functionally-polarized material(s).

The compositions disclosed herein have the ability and/or capability to overcome mechanical, electrical, chemical barriers as well as voids, defects or errors in material(s), substrate(s), tissue(s) because the compositions disclosed herein have the ability to recognize, sense, calculate, coordinate and self-determine the response to the environment or system into which the compositions are placed.

The compositions disclosed herein have the ability to self-propagate, differentiate, adapt, evolve, replicate, migrate, self-synthesize, self-modulate, and self-regulate all elements in the compositions, as well as impact the environment and/or system in which the compositions are placed.

The compositions disclosed herein have the ability to alter the environment in which they are placed or materialized within by directing and/or coordinating the: synthesis, alteration, modification, modulation, regulation, assembly, or destruction of materials including, but not limited to:
chemical, electrochemical and/or electrical environments;
genomic, epigenomic, transcriptomic, epitrascriptomic, proteomic, epiproteomic materials;
sub-cellular organelles or sub-cellular structures as well as derivatives of such structures;
intracellular and/or extracellular matrices, scaffolds, particles, fibers and or structural elements;
anabolic, catabolic and/or metabolic processes and materials, as well as derivatives of such materials;
material mechanics, material forces, material kinetics and/or material thermodynamics;
other living and/or living materials or cellular entities;
tissue and/or organ systems;
cell and/or cellular systems; and
composite systems.

Aspects of the present disclosure also relate to methods of preparing compositions disclosed herein. Furthermore, aspects of the present disclosure relate to methods of treatment using the compositions disclosed herein.

### Compositions

Disclosed herein are compositions comprising synthesized structure(s) of an aggregate of dynamic, reactive three-dimensionally interfaced cellular entities which contain both interactive, living core potent cellular entities and supportive entities. More particularly, the core potent cellular entities are interfaced with supportive entities (e.g., cellular progeny) in an interface-derived orientation that directs the formation of functional, polarized, self-organizing material(s).

In an embodiment, a composition comprises a stimulated heterogeneous mammalian tissue interface cell aggregate that is capable of producing functional polarized tissue when administered to a subject in need thereof.

In an embodiment, the stimulated heterogeneous mammalian tissue interface cell aggregate is derived from an osseous tissue interface. The osseous tissue interface can be selected from a peri-cortical tissue interface, a peri-lamellar tissue interface, a peri-trabecular tissue interface, a cortico-cancellous tissue interface, or a combination thereof.

In an embodiment, the stimulated heterogeneous mammalian tissue interface cell aggregate is derived from a cutaneous tissue interface. In an embodiment, the stimulated heterogeneous mammalian tissue interface cell aggregate is derived from a musculoskeletal tissue interface. In an embodiment, the stimulated heterogeneous mammalian tissue interface cell aggregate is derived from a smooth muscle tissue interface. In an embodiment, the stimulated heterogeneous mammalian tissue interface cell aggregate is derived from a cardiac muscle tissue interface. In an embodiment, the stimulated heterogeneous mammalian tissue interface cell aggregate is derived from a cartilage tissue interface. In an embodiment, the stimulated heterogeneous mammalian tissue interface cell aggregate is derived from an adipose tissue interface. In an embodiment, the stimulated heterogeneous mammalian tissue interface cell aggregate is derived from gastrointestinal tissue interface. In an embodiment, the stimulated heterogeneous mammalian tissue interface cell aggregate is derived from a pulmonary tissue interface. In an embodiment, the stimulated heterogeneous mammalian tissue interface cell aggregate is derived from an esophageal tissue interface. In an embodiment, the stimulated heterogeneous mammalian tissue interface cell aggregate is derived from a gastric tissue interface. In an embodiment, the stimulated heterogeneous mammalian tissue interface cell aggregate is derived from a renal tissue interface. In an embodiment, the stimulated heterogeneous mammalian tissue interface cell aggregate is derived from a hepatic tissue interface. In an embodiment, the stimulated heterogeneous mammalian tissue interface cell aggregate is derived from a pancreatic tissue interface. In an embodiment, the stimulated heterogeneous mammalian tissue interface cell aggregate is derived from a blood vessel tissue interface. In an embodiment, the stimulated heterogeneous mammalian tissue interface cell aggregate is derived from a lymphatic tissue interface. In an embodiment, the stimulated heterogeneous mammalian tissue interface cell aggregate is derived from a central nervous tissue interface. In an embodiment, the stimulated heterogeneous mammalian tissue interface cell aggregate is derived from a urogenital tissue interface. In an embodiment, the stimulated heterogeneous mammalian tissue interface cell aggregate is derived from a glandular tissue interface. In an embodiment, the stimulated heterogeneous mammalian tissue interface cell aggregate is derived from a dental tissue interface. In an embodiment, the stimulated heterogeneous mammalian tissue interface cell aggregate is derived from a peripheral nerve tissue interface. In an embodiment, the stimulated heterogeneous mammalian tissue interface cell aggregate is derived from a birth tissue interface. In an embodiment, the stimulated heterogeneous mammalian tissue interface cell aggregate is derived from an optic tissue interface. In an embodiment, the stimulated heterogeneous mammalian tissue interface cell aggregate comprises living core potent cellular entities and supportive entities. The living core potent cellular entities can express RNA transcripts and/or polypeptides of one or more Leucine Rich Repeat Containing G Protein-Coupled Receptors selected from the group consisting of LGR4, LGR5, LGR6, and any combination thereof. The living core potent cellular entities can express RNA transcripts and/or polypeptides of one or more of Pax 7, Pax 3, MyoD, Myf 5, keratin 15, keratin 5, cluster of differentiation 34 (CD34), Sox9, c-Kit+, Sca-1+, or any combination thereof.

The supportive entities can comprise mesenchymal derived cellular populations. The supportive entities can comprise cellular populations, extracellular matrix elements, or a combination thereof. The extracellular matrix elements can comprise one or more of hyaluronic acid, elastin, collagen, fibronectin, laminin, extracellular vesicles, enzymes, and glycoproteins.

In an embodiment, the stimulated heterogeneous mammalian tissue interface cell aggregate shows increased expression levels of parathyroid hormone compared to that observed in native osseous tissue. The stimulated heterogeneous mammalian tissue interface cell aggregate can show from 10-fold to 15-fold increase in expression levels of parathyroid hormone compared to that observed in native osseous tissue.

In an embodiment, the stimulated heterogeneous mammalian tissue interface cell aggregate shows increased expression levels of TLR4 compared to that observed in native osseous tissue.

In an embodiment, the stimulated heterogeneous mammalian tissue interface cell aggregate shows increased expression levels of thymidine phosphorylase compared to that observed in native osseous tissue. The stimulated heterogeneous mammalian tissue interface cell aggregate can show from 100-fold to 200-fold increase in expression levels of thymidine phosphorylase compared to that observed in native osseous tissue.

In an embodiment, the functional polarized tissue shows decreased expression levels of one or more of IL2, MYOSIN2, ITGB5, and STAT3 compared to that observed in native osseous tissue. In an embodiment, the functional polarized tissue shows at least 98% similarity in gene expression compared to native osseous tissue.

The composition can further comprise a delivery substrate. In an embodiment, the delivery substrate comprises a scaffold.

In an embodiment, the stimulated heterogeneous mammalian tissue interface cell aggregate has a diameter of about 50 μm. In an embodiment, the stimulated heterogeneous mammalian tissue interface cell aggregate has a diameter of about 40-250 μm, for example, about 50-250 μm, about 75-250 μm, about 100-250 μm, about 125-250 μm, about 150-250 μm, about 175-250 μm, about 200-250 μm, or about 225-250 μm.

In an embodiment, a composition comprises at least a portion of a mammalian material interface comprising core potent cellular entities and supportive entities. The composition is capable of assembling functional material.

In an embodiment, the mammalian material interface is derived from a cutaneous tissue interface. In an embodiment, the mammalian material interface is derived from an osseous tissue interface. In an embodiment, the mammalian material interface is derived from a musculoskeletal tissue interface. In an embodiment, the mammalian material interface is derived from a smooth muscle tissue interface. In an embodiment, the mammalian material interface is derived from a cardiac muscle tissue interface. In an embodiment, the mammalian material interface is derived from a cartilage tissue interface. In an embodiment, the mammalian material interface is derived from an adipose tissue interface. In an embodiment, the mammalian material interface is derived from gastrointestinal tissue interface. In an embodiment, the mammalian material interface is derived from a pulmonary tissue interface. In an embodiment, the mammalian material interface is derived from an esophageal tissue interface. In an embodiment, the mammalian material interface is derived from a gastric tissue interface. In an embodiment, the mammalian material interface is derived from a renal tissue interface. In an embodiment, the mammalian material interface is derived from a hepatic tissue interface. In an embodiment, the mammalian material interface is derived from a pancreatic tissue interface. In an embodiment, the mammalian material interface is derived from a blood vessel tissue interface. In an embodiment, the mammalian material interface is derived from a lymphatic tissue interface. In an embodiment, the mammalian material interface is derived from a central nervous tissue interface. In an embodiment, the mammalian material interface is derived from a urogenital tissue interface. In an embodiment, the mammalian material interface is derived from a glandular tissue interface. In an embodiment, the mammalian material interface is derived from a dental tissue interface. In an embodiment, the mammalian material interface is derived from a peripheral nerve tissue interface. In an embodiment, the mammalian material interface is derived from a birth tissue interface. In an embodiment, the mammalian material interface is derived from an optic tissue interface.

Exemplary core potent cellular entities include stem cells, progenitor cells, and transit-amplifying cells. Core potent cellular entities suitable for use in the compositions disclosed herein can be identified or established by, for example, identifying certain sub-cellular sequence markers (i.e., DNA, RNA, and proteins). In particular embodiments, the compositions disclosed herein comprise aggregates of interfaced core potent cellular entities and supportive entities, which core potent cellular entities express a sequence of the Leucine Rich Repeat Containing G Protein-Coupled Receptor (LGR). In embodiments, core potent cellular entities express a sequence of LGR4, a sequence of LGR5, a sequence of LGR6, or combinations thereof.

Methods of identifying core potent cellular entities are known in the art. Core potent cellular entities can be identified by, for example, electron microscopy, phase-contrast microscopy on single myofiber explants or fluorescence microscopy. For example, in vivo satellite cell populations can be visualized using developed bioluminescence imaging techniques. For example, satellite cells can be identified using electronic microscopy based on their “wedged” appearance and morphological characteristics including large nuclear to cytoplasmic ratio, few organelles, small nucleus, and condensed interphase chromatin. In vivo, satellite cells can also be identified by fluorescence microscopy, using including one or more transcription factors and/or cell membrane proteins as biomarkers such as Pax 7, Pax 3, MyoD, and Myf 5.

As disclosed herein, neo-generative, regenerative polarity and/or organized formation of materials can be induced and propagated by placing, deploying and/or materializing such described composition within a target material and/or substrate.

The interface(s) of the disclosed compositions relate to direct or indirect forms of cell-to-cell, cell-to-intracell, cell-to-substrate, cell-to-agent, cell-to-material factor(s), cell-to-environment, cell-to-system, cell-to-interactome at which such interface permits the contact, communication, modulation, regulation, initiation, effect, response, chemical/mechanical interaction, transfer of materials and/or energy to alter or impact the environment or system in which the composition is delivered or deployed.

Such interface relates to direct or indirect forms of cell-to-ECI (extra-cellular interactome) or extracellular substrate contact, communication, effect, response, chemical, and/or mechanical interactions (e.g., molecules, growth factors, peptides, metabolites, DNA/RNA, micro-organisms, chemical gradients, agent gradients, electrical gradients, photons and/or energy).

In embodiments, the compositions described herein are capable of assembling functional material (e.g., functional tissue) in vivo.

In embodiments, the compositions described herein are capable of assembling functional material (e.g., functional tissue) ex vivo.

In embodiments, the compositions described herein are capable of assembling functional material (e.g., functional tissue) in vitro.

In embodiments, the compositions described herein are capable of assembling functional material (e.g., functional polarized tissue) in complex or composite systems.

As used herein, the “administration” of a composition to a subject includes any route of introducing or delivering to a subject a composition to perform its intended function. Administration can be carried out by any suitable route, including but not limited to, by transplantation, orally, intranasally, parenterally (intravenously, intramuscularly, intraperitoneally, or subcutaneously), rectally, intrathecally, or topically. Administration includes self-administration and the administration by another.

As used herein, “core potent cellular entities” refer to cellular entities that are capable of intercellular communication, migration, chemotaxis, proliferation, differentiation, transdifferentiation, dedifferentiation, transient amplification, asymmetrical division and include stem cells, progenitor cells, and transit-amplifying cells. Core potent cellular entities may be identified or established by, for example, assaying for certain sub-cellular biomarkers (i.e., DNA, RNA, and proteins). In some embodiments, core potent cellular entities express RNA transcripts and/or polypeptides of one or more Leucine Rich Repeat Containing G Protein-Coupled Receptors (LGR), such as LGR4, LGR5, LGR6, or combinations thereof. Additionally or alternatively, in some embodiments, core potent cellular entities express RNA transcripts and/or polypeptides of one or more of Pax 7, Pax 3, MyoD, Myf 5, keratin 15, keratin 5, cluster of differentiation 34 (CD34), Sox9, c-Kit+, Sca-1+, and any combination thereof. Additional examples of biomarkers for core potent cellular entities are described in Wong et al., International Journal of Biomaterials, vol. 2012, Article ID 926059, 8 pages, 2012.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease or condition and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein.

As used herein, the term “effective reactive stimulant” refers to any additive that activates cells, cell populations, cellular tissues, and aggregates of heterogeneous mammalian tissue interface cells, which can activate or alter the physiology of the above said cells and can be performed by one or a combination of signals including chemokine receptor binding, paracrine receptor binding, cell membrane alteration, cytoskeletal alteration, physical manipulation of the cell, altering physiological gradients, altering temperature, small molecule interactions, introduction of nucleotides and ribonucleotides such as small inhibitory RNAs.

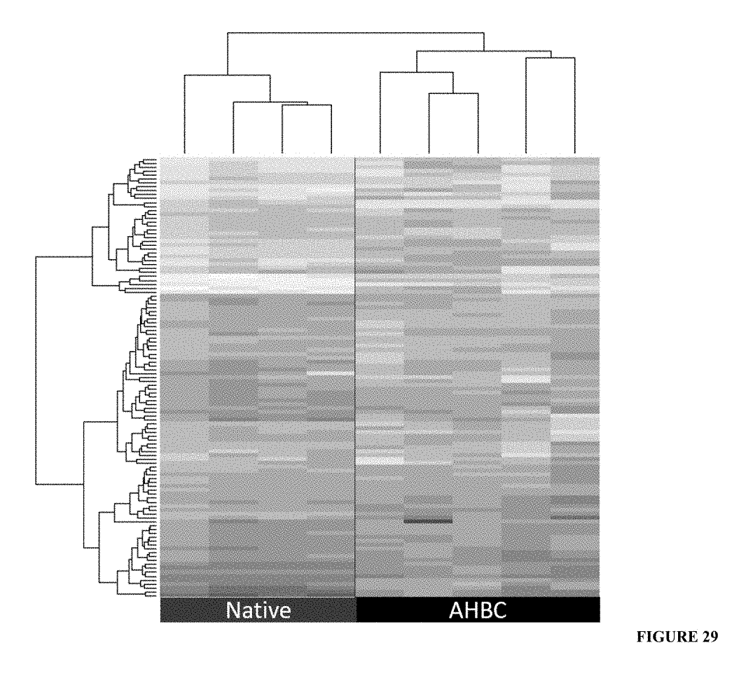

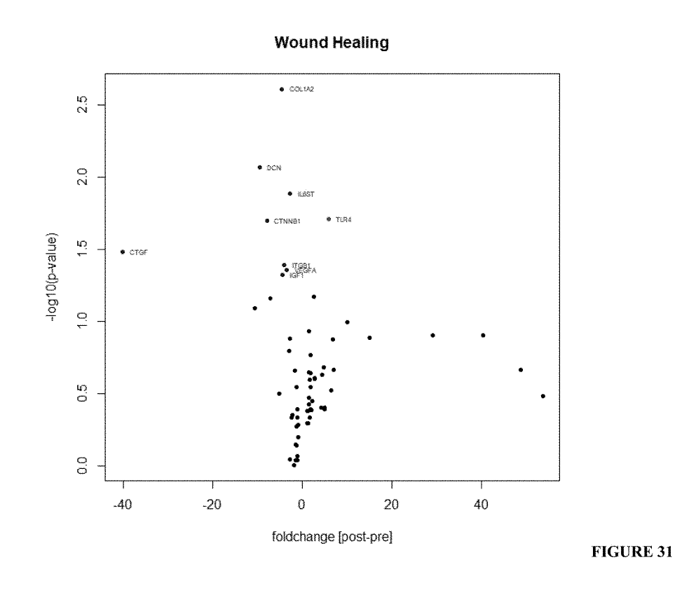

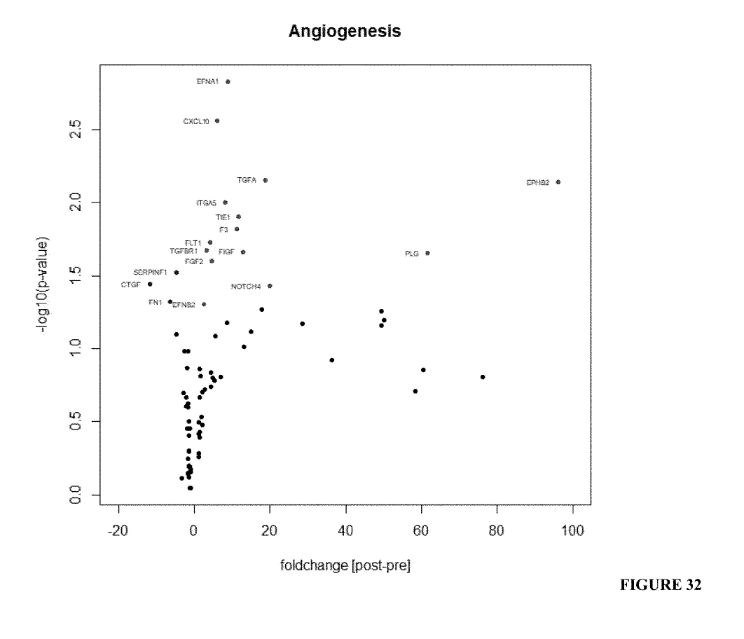

As used herein, “stimulated” refers to activating (e.g., changing) the physiological state of an aggregate of heterogeneous mammalian tissue interface cells that can be performed by one or a combination of signals including electrical stimulation, oxygen gradient, chemokine receptor binding, paracrine receptor binding, cell membrane alteration, cytoskeletal alteration, physical manipulation of cells, alteration of physiological gradients, alteration of temperature, small molecule interactions, introduction of nucleotides and ribonucleotides such as small inhibitory RNAs, which are sufficient to induce one or more of the following phenotypes/outcomes: altered gene expression (see, e.g., heat maps and volcano plots in FIGS. 29-34), altered protein translation, altered intracellular and intercellular signaling, altered binding of vesicles to membranes, altered ATP production and consumption, and altered cellular mobility.

As used herein “supportive entities” refer to non-stem cell populations (e.g., supportive cellular entities) and/or extracellular matrix materials that provide structural and biochemical support for core potent cellular entities. In some embodiments, supportive cellular entities may comprise proliferating and/or differentiating cells. Additionally or alternatively, in some embodiments, supportive cellular entities may be identified by expression of biomarkers such as BMPr1a, BMP2, BMP6, FGF, Notch receptors, Delta ligands, CXCL12, Sonic Hedge Hog, VEGF, TGFβ, Wnt, HGF, NG2, and alpha smooth muscle actin. In some embodiments, the supportive cellular entities comprise mesenchymal derived cellular populations.

As used herein, a “therapeutically effective amount” of a composition refers to composition levels in which the physiological effects of a disease or condition are ameliorated or eliminated. A therapeutically effective amount can be given in one or more administrations

As used herein, “extracellular matrix” and “extracellular matrix elements” refer to extracellular macromolecules, such as hyaluronic acid, elastin, collagen, fibronectin, laminin, extracellular vesicles, enzymes, and glycoproteins, that are organized as a three-dimensional network to provide structural and biochemical support for surrounding cells.

As used herein, the term “AHBC” refers to an autologous homologous bone construct. As used herein, the term “AHLC” refers to an autologous homologous liver construct. As used herein, the term “AHSC” refers to an autologous homologous skin construct.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “functional material”, “functional tissue”, and “functional polarized tissue” refers to an ensemble of cells and their extracellular matrix having the same origin and executing biological functions similar to that observed in the native counterpart tissue. In some embodiments, the “functional material”, “functional tissue”, or “functional polarized tissue” exhibits characteristics such polarity, density, flexibility, etc., similar to that observed in the native counterpart tissue.

Disclosed herein are compositions which develop and promote material and system polarity.

As used herein the term “material interface” refers to the region, area and/or location where two or more different or distinguishable cells approach, contact, merge, integrate, incorporate, unite, coalesce, combine, compound, fuse, abut, touch, border, meld, communicate, synapse, junction, interact, share, aggregate, connect, penetrate, surround, or form with each other in an environment and/or system which may or may not contain other materials, substrates or factors. This other environment(s) and/or system(s) may be used to interact with the compositions disclosed herein.

As used herein, a “tissue interface” refers to a location at which independent and optionally unrelated tissue systems interact and communicate with each other. In some embodiments, components of a tissue interface currently promote/promoted histogenesis and cell development and/or metabolism, including but not limited to proliferation, differentiation, migration, anabolism, catabolism, stimulation, or at least one of intracellular, intercellular, extracellular, transcellular, and pericellular communication or any combination thereof.

Compositions disclosed herein are comprised of a complete interface compartment or a sub-compartment interface which can then be utilized to synthesize a complete interface. A complete interface compartment refers to the content materials located within said region, area and/or location which when processed as disclosed herein would supply or could supply, through further processing, those materials necessary for the development of the compositions disclosed herein. As described in more detail below, for each material substrate and/or tissue of interest, a complete interface compartment would include those essential layers of that tissue that contribute to its unique function.

A sub-compartment interface also refers to the content materials located within said region, area and/or location which when processed as disclosed herein would supply or could supply, through further processing, those materials necessary for the development of the compositions disclosed herein. A sub-interface refers to a portion of a complete interface.

In the case of cutaneous tissue, a cutaneous tissue interface can include epidermal-dermal interface, papillary-reticular dermal interface, dermal-hypodermal interface, hypodermal-subdermal interface, appendage-substrate interface and combinations thereof.

In the case of osseous tissue, an osseous tissue interface can include a peri-cortical tissue interface, a peri-lamellar tissue interface, a peri-trabecular tissue interface, a cortico-cancellous tissue interface, and combinations thereof.

In the case of musculoskeletal tissue, a musculoskeletal tissue interface can include a myo-epimysial tissue interface, a myo-perimysial tissue interface, a myo-endomysial tissue interface, a myo-fascial tissue interface, a tendon-muscle tissue interface, a tendon-bone tissue interface, a ligament-bone tissue interface, and combinations thereof.

In the case of smooth muscle tissue, a smooth muscle tissue interface can include a perivascular tissue interface, a perivisceral tissue interface, a perineural tissue interface, and combinations thereof.

In the case of cardiac muscle tissue, a cardiac muscle tissue interface can include an endocardial-myocardial tissue interface, a myocardial-epicardial tissue interface, an epicardial-pericardial tissue interface, a pericardial-adipose tissue interface, and combinations thereof.

In the case of cartilage tissue, a cartilage tissue interface can include a chondrial-perichondrial tissue interface, a chondrial-endochondrial tissue interface, an endochondrial-subchondral bone interface, a chondrial-endochondrial bone interface, an endochondrial-subchondral bone interface, and combinations thereof.

In the case of adipose tissue, an adipose tissue interface can include an adipo-perivascular tissue interface, an adipo-peristromal tissue interface, and combinations thereof.

In the case of gastrointestinal tissue, a gastrointestinal tissue interface can include a mucosal-submucosal tissue interface, a sub-mucosal-muscularis tissue interface, a muscularis-serosal tissue interface, a serosal-mesentery tissue interface, a myo-neural tissue interface, a submucosal-neural tissue interface, and combinations thereof.

In the case of pulmonary tissue, a pulmonary tissue interface can include a mucosal-submucosal tissue interface, a sub-mucosal-muscularis tissue interface, a sub-mucosal-cartilage tissue interface, muscular-adventitial tissue interface, a ductal-adventitial tissue interface, a parenchymal-serosal tissue interface, a serosal-mesentery tissue interface, a myo-neural tissue interface, a submucosal-neural tissue interface, and combinations thereof.

In the case of esophageal tissue, an esophageal tissue interface can include a mucosal-submucosal tissue interface, a sub-mucosal-muscularis tissue interface, a muscularis-adventitial tissue interface, a myo-neural tissue interface, a submucosal-neural tissue interface, and combinations thereof.

In the case of gastric tissue, a gastric tissue interface can include a mucosal-submucosal tissue interface, a sub-mucosal-muscularis tissue interface, a muscularis-serosal tissue interface, a myo-neural tissue interface, a submucosal-neural tissue interface, and combinations thereof.

In the case of renal tissue, a renal tissue interface can include a capsule-cortical tissue interface, a cortical-medullary tissue interface, a neuro-parenchymal tissue interface, and combinations thereof.

In the case of hepatic tissue, a hepatic tissue interface can include ductal epithelial-parenchymal tissue interface, a capsular-parenchymal tissue interface, and combinations thereof.

In the case of pancreatic tissue, a pancreatic tissue interface can include a ductal epithelial-parenchymal tissue interface, a glandular epithelial-parenchymal tissue interface, and combinations thereof.

In the case of blood vessels, a blood vessel tissue interface can include an endothelial-tunica tissue interface, a tunica-tunica tissue interface, and combinations thereof.

In the case of lymphatic tissue, a lymphatic tissue interface can include a cortico-medullary tissue interface, a medullary-capsule tissue interface, a capsule-pulp tissue interface, and combinations thereof.

In the case of central nervous tissue, a central nervous tissue interface can include a dural-cortex tissue interface, a cortical grey matter-medullary white matter tissue interface, a meningeal-neural tissue interface, and combinations thereof.

In the case of urogenital tissue, a urogenital tissue interface can include an epithelial-mucosal tissue interface, a mucosal-muscular tissue interface, a muscular-adventitial tissue interface, a corporal-vascular tissue interface, a corporal-muscular tissue interface, and combinations thereof.

In the case of glandular tissue, a glandular tissue interface can include an epithelial-parenchymal tissue interface.

In the case of dental tissue, a dental tissue interface can include a dentin-pulp tissue interface.

In the case of peripheral nerve tissue, a peripheral nerve tissue interface can include an epineural-perineural tissue interface, a perineural-endoneural tissue interface, an endoneural-axonal, and combinations thereof.

In the case of birth tissue, a birth tissue interface can include an amnion-fluid tissue interface, an epithelial-sub-epithelial tissue interface, an epithelial-stroma tissue interface, a compact-fibroblast tissue interface, a fibroblast-intermediate tissue interface, an intermediate-reticular tissue interface, an amnio-chroion tissue interface, a reticular-trophoblast tissue interface, a trophoblast-uterine tissue interface, a trophoblast-decidua tissue interface, and combinations thereof.

In the case of optic tissue, an optic tissue interface can include an epithelial-membrane tissue interface, a membrane-stroma tissue interface, a stromal-membrane tissue interface, a membrane-endothelial tissue interface, an endothelial-fluid tissue interface, a scleral-choroid tissue interface, a choroid-epithelial tissue interface, an epithelial-segmental photoreceptor tissue interface, a segmental photoreceptor-membrane tissue interface, a membrane-outer nuclear layer tissue interface, an outer nuclear layer-outer plexiform tissue interface, an outer plexiform-inner plexiform tissue interface, an inner plexiform-ganglion tissue interface, a ganglion-neural fiber tissue interface, a neural fiber tissue interface-membrane tissue interface, a membrane-fluid tissue interface, and combinations thereof.

Supportive entities can include cellular and non-cellular materials. In one embodiment, the supportive entities include cellular entities which comprise non-stem interfaced cellular populations. In another embodiment, the supportive materials include cellular entities which comprise interfaced cellular progeny populations and/or differentiating entities.

In embodiments, the supportive entities comprise mesenchymal derived cellular populations. In embodiments, the supportive entities comprise cellular populations, extracellular matrix elements, or combinations thereof.

The composition can also include a delivery substrate. The delivery substrate can be selected from a variety of carrier mediums which include but are not limited to molecules, materials, fluids, scaffolds, matrices, particles, cells, fibers, sub-cellular structures, biologics, devices and/or combinations thereof. In an embodiment, the delivery substrate is selected from a scaffold, matrix, particle, cells, fiber, or combinations thereof.

The composition can also comprise a supplement selected from a growth factor, an analyte, a LGR interactive element, or combinations thereof. The analyte can be selected from a migratory analyte, a recruiting analyte, a stimulatory agent, an inhibitory agent, or combinations thereof.

Alternatively, the disclosed compositions can act as a delivery, deployment and/or carrier substrate and/or vector for other forms of active or acting matter.

Alternatively, the disclosed composition can be used as a barrier or covering of other materials requiring such action.

Alternatively, the disclosed composition can be used to enhance other materials in which the composition interacts or interfaces with in direct and indirect forms.

In embodiments, the compositions disclosed herein further comprise a system capable of purposeful actions by which agents, substances, materials, substrates, factors, analytes, supplements, molecules are developed from the composition described herein which may act locally, system-wide, on other forms of matter and/or within an auto-reactive manner.

In embodiments, the compositions disclosed herein further comprise a material which develops and/or acts to enhance the viability, propagation, proliferation, differentiation, migration, stimulation, alteration, augmentation, modulation of systems and entities in communication with the said composition disclosed herein.

In embodiments, the compositions disclosed herein further comprise a material which develops and/or acts to enhance the regulation, inhibition, stagnation, termination, destruction, obliteration, cessation of systems and entities in communication with the said composition disclosed herein.

In embodiments, the composition may be placed directly into living systems, partial living systems, non-living systems, artificial systems and/or synthetic supportive systems which permit the material(s) to persist and/or propagate.

In embodiments, the composition may be altered, changed, regulated, manipulated, adjusted, modified, transformed, converted, mutated, reconstructed, evolved, adapted, integrated and/or subtracted from and/or added to other material(s) directly and/or indirectly so as to change the primary material(s) in function, appearance, structure, makeup, behavior and/or existence within such systems or environments.

### Methods of Production

The present disclosure also provides a method for producing a composition as disclosed herein. The method involves isolating at least a portion of a mammalian material interface comprising core potent cellular entities and supportive entities. The method further involves developing a reactive and stimulated interface to provide the composition. The composition is capable of assembling functional material.

In an embodiment, the mammalian material interface is a cutaneous tissue interface. In an embodiment, the mammalian material interface is an osseous tissue interface. In an embodiment, the mammalian material interface is a musculoskeletal tissue interface. In an embodiment, the mammalian material interface is a smooth muscle tissue interface. In an embodiment, the mammalian material interface is a cardiac muscle tissue interface. In an embodiment, the mammalian material interface is a cartilage tissue interface. In an embodiment, the mammalian material interface is an adipose tissue interface. In an embodiment, the mammalian material interface is a gastrointestinal tissue interface. In an embodiment, the mammalian material interface is a pulmonary tissue interface. In an embodiment, the mammalian material interface is an esophageal tissue interface. In an embodiment, the mammalian material interface is a gastric tissue interface. In an embodiment, the mammalian material interface is a renal tissue interface. In an embodiment, the mammalian material interface is a hepatic tissue interface. In an embodiment, the mammalian material interface is a pancreatic tissue interface. In an embodiment, the mammalian material interface is a blood vessel tissue interface. In an embodiment, the mammalian material interface is a lymphatic tissue interface. In an embodiment, the mammalian material interface is a central nervous tissue interface. In an embodiment, the mammalian material interface is a urogenital tissue interface. In an embodiment, the mammalian material interface is a glandular tissue interface. In an embodiment, the mammalian material interface is a dental tissue interface. In an embodiment, the mammalian material interface is a peripheral nerve tissue interface. In an embodiment, the mammalian material interface is a birth tissue interface. In an embodiment, the mammalian tissue interface is an optic tissue interface. Exemplary tissue interfaces are described above.

In embodiments, the supportive entities comprise mesenchymal derived cellular populations. In embodiments, the supportive entities are selected from cellular populations, extracellular matrix elements, or combinations thereof.

The materials for the development of the disclosed compositions can be obtained from a cell-tissue environment and/or system(s) in either complete interface compartments or sub-compartment interfaces. Once located, the population containing the core potent cellular entities and supportive entities surrounding the mammalian material interface can be obtained through a variety of methods which would be understood by one of ordinary skill in the art. Such methods include, but are not limited to, harvest, biopsy, punch, cleavage, restriction, digestion, extraction, excision, disassociation, separation, removal, partition, and/or isolation. Once the cellular population containing the core potent cellular entities and the supportive entities are obtained, the mammalian material interface or sub-interface is disrupted so as to disrupt organization of the material without complete destruction of the material and obtain minimal polarization. As used herein, “minimal polarization” refers to the degree of polarization achieved by artificial manipulation of biological material that is necessary for a unit of tissue to be capable of assembling functional polarized tissue. Artificial manipulation may be achieved using mechanical, chemical, enzymatic, energetic, electrical, biological and/or other physical methods.

A variety of disruption methods would be understood to those of skill in the art, including but not limited to, mechanical, chemical, enzymatic, energetic, electrical, biological and/or physical mechanisms. Such disruption develops a reactive and stimulated interface.

Also disclosed herein is a method for preparing a composition comprising a stimulated heterogeneous mammalian tissue interface cell aggregate that is capable of producing functional polarized tissue when administered to a subject in need thereof. In some embodiments, the method comprises isolating at least a portion of a mammalian material interface to obtain a heterogeneous mammalian tissue interface cell aggregate, wherein the mammalian material interface comprises heterogeneous mammalian tissue interface cells; and stimulating the heterogeneous mammalian tissue interface cells.

In embodiments, stimulating comprises mechanical stimulation, chemical stimulation, enzymatic stimulation, energetic stimulation, electrical stimulation, biological stimulation, or any combination thereof. In embodiments, the stimulating comprises dissociation, dissection, cutting, shearing, vortexing, or any combination thereof. In embodiments, chemical or biological stimulation comprises at least one of chemokine receptor binding, paracrine receptor binding, cell membrane alteration, cytoskeletal alteration, alteration of physiological gradients, addition of small molecules or addition of nucleotides and ribonucleotides.

In embodiments, the disrupted interface material (i.e., the reactive and stimulated interface) can then be collected and/or segregated. This can be accomplished in a variety of ways known to skilled artisans including, but not limited to functional filtration, fractionation, capture selection, centrifugation, enrichment, ancillary reduction, separation, gradation, partition, precipitation of said material(s).

In embodiments, the non-interface material (remaining from the mammalian specimen material from which at least a portion of the mammalian material interface is isolated) can then be collected and/or segregated. Those skilled in the art will appreciate that this can be accomplished in a variety of ways including, but not limited to functional filtration, fractionation, capture selection, centrifugation, enrichment, ancillary reduction, separation, gradation, partition, precipitation of said material(s).

In embodiments, the disrupted interface material and non-interface material are combined, in whole or in part, to create a composition capable of assembling functional material. Alternatively, the disrupted interface material can be used alone (i.e., without the non-interface material). The reactive and stimulated interface achieved by ex vivo or artificial stimulation provides the composition that is capable of assembling functional material. In embodiments, the composition may also be placed directly into living systems, partial living systems, and/or synthetic supportive systems which permit the material(s) to persist and/or propagate.

In embodiments, a delivery substrate may be added to the composition. The delivery substrate may encompass a solid, semi-solid, liquid, semi-liquid, fluid, particle, fiber, scaffold, matrix, molecule, substrate, material, cellular entity, tissue entity, device, biologic, therapeutic, macromolecule, chemical, agent, organism, media and/or synthetic substance, and combinations thereof. In an embodiment, the delivery substrate is selected from a scaffold, matrix, particle, cells, fiber, or combinations thereof.

In embodiments, the method can further involve adding a supplement selected from a growth factor, an analyte, a LGR interactive element, or combinations thereof. The analyte can be selected from a migratory analyte, a recruiting analyte, a stimulatory agent, an inhibitory agent, or combinations thereof.

During stimulating events of the interface and non-interface material(s), an associated material agent is produced and/or generated. In embodiments, this agent may be combined with the reactive and stimulated interface and non-interface material to generate a composition capable of assembling functional material. Alternatively, such agent may be used independently. As another alternative, such agent may be added to other matter or combined within other systems.

In embodiments, the composition produced by the method described herein is capable of assembling functional material in vivo. In embodiments, the composition produced by the method described herein is capable of assembling functional material ex vivo. In embodiments, the composition produced by the method described herein is capable of assembling functional material in vitro. One of ordinary skill in the art would recognize appropriate and conventional growth media to use in conjunction with the compositions disclosed herein in order to assemble functional polarized tissue ex vivo or in vitro.

The composition can then be subject to stabilization, preservation, immortalization, cultivation, expansion, or fractional distribution by methods understood by one of ordinary skill in the art.

The composition can also be cryopreserved or lyophilized (i.e., freeze-dried) according to known methods. Methods of lyophilizing may include one or more pretreatments (e.g., concentrating the composition; adding a cryoprotectant to the composition; increasing the surface area of the composition; freezing the composition; and drying the composition such as, for example, exposing the composition to a reduced atmospheric pressure to result in sublimation of the water present in the composition).

### Methods of Use

The disclosed compositions derived from each tissue can be used across a variety of applicable fields including but limited to medicine/research/regenerative medicine/tissue engineering/food/manufacturing/military through the delivery, deployment, coupling, integration, combined synthesis, addition of the disclosed compositions to some form of an integrated type of delivery system, platform or composite arrangement which includes but is not limited to a vector, substrate, fluid, support, scaffold, matrix, device, biologic, cell, tissue, polymers, molecules, particles, fibers, therapies for direct or indirect applications.

Also disclosed herein is a method for treating a subject in need of tissue repair comprising administering to a subject an effective amount of a composition as disclosed herein.

Also disclosed herein is a method for promoting tissue (e.g., osseous tissue, cutaneous tissue, musculoskeletal tissue, smooth muscle tissue, cardiac muscle tissue, cartilage tissue, adipose tissue, gastrointestinal tissue, pulmonary tissue, esophageal tissue, gastric tissue, renal tissue, hepatic tissue, pancreatic tissue, blood vessel tissue, lympatic tissue, central nervous tissue, urogenital tissue, glandular tissue, dental tissue, peripheral nerve tissue, birth tissue, or optic tissue) regeneration in a subject in need thereof comprising administering to the subject an effective amount of a composition as disclosed herein.

In embodiments, the subject is suffering from a degenerative tissue (e.g., osseous tissue, cutaneous tissue, musculoskeletal tissue, smooth muscle tissue, cardiac muscle tissue, cartilage tissue, adipose tissue, gastrointestinal tissue, pulmonary tissue, esophageal tissue, gastric tissue, renal tissue, hepatic tissue, pancreatic tissue, blood vessel tissue, lympatic tissue, central nervous tissue, urogenital tissue, glandular tissue, dental tissue, peripheral nerve tissue, birth tissue, or optic tissue) disease. In embodiments, the degenerative bone disease is osteoarthritis or osteoporosis. In embodiments, the subject is suffering from a bone fracture or break. In embodiments, the fracture is a stable fracture, an open compound fracture, a transverse fracture, an oblique fracture, or a comminuted fracture.

The compositions disclosed herein can serve as a substitute for scaffold or void fillers or in conjunction with other devices to promote tissue healing, fill voids, maintain essential structure, and bridge separate tissue surfaces via their biologic and mechanical characteristics. Thus, the compositions disclosed herein can be applied in graft procedures including, but not limited to, orthopedic surgery, neurological surgery, plastic surgery, dental surgery, and dermatologic surgery.

Also disclosed herein is a method for treating a subject in need of tissue repair comprising administering to the subject an effective amount of a composition comprising a stimulated heterogeneous mammalian tissue interface cell aggregate that is capable of producing functional polarized tissue when administered to a subject in need thereof, wherein administration of the composition results in an increase in at least one of parathyroid hormone, TLR4, thymidine phosphorylase in the subject compared to that observed prior to administration.

Also disclosed herein are methods of treating a disease or disorder of tissue that results in loss or destruction of tissue or, alternatively, results in failure of tissue formation or, yet alternatively, causes formation of abnormal tissue. Disclosed herein is a method of treating a disease or disorder of tissue, comprising administering a composition disclosed herein to a target site of a subject in need thereof, wherein the disease or disorder of the tissue results in: (i) loss or destruction of the tissue; (ii) failure of formation of the tissue; or (iii) formation of abnormal tissue.

Also disclosed herein is a method of treating a disease or disorder of tissue, comprising transplanting a composition disclosed herein at a target site of a subject in need thereof, wherein the disease or disorder of the tissues results in: (i) loss or destruction of the tissue; (ii) failure of formation of the tissue; or (iii) formation of abnormal tissue.

Similarly, disclosed herein is a method of treating a disease or disorder of the tissue, comprising implanting a composition disclosed herein at a target site of a subject in need thereof, wherein the disease or disorder of the tissue results in: (i) loss or destruction of the tissue;

(ii) failure of formation of the tissue; or (iii) formation of abnormal tissue.

Also disclosed herein is a kit comprising a composition as disclosed herein and instructions for use.

As used herein, the term “subject” as used herein refers to a mammal. In embodiments, the mammal is a human. In other embodiments, the mammal is a non-human animal. The mammal can be, for example, selected from rats, mice, pigs, horses, goats, sheep, rabbits, dogs, cats, primates, cows, oxen, camels, asses, guinea pigs, or bison.

As used herein, the term “target site” or “target” refers to a location within, on, or adjacent to tissue on which the composition seeks to directly or indirectly impact, act on, or change.

“Treatment” and “treating” as used herein does not require complete cure of the disease or disorder or complete resolution of the symptoms of the disease or disorders (e.g., complete formation or reconstruction of functional tissue). The mode of administration may be any suitable mode. Representative, non-limiting modes of administration include placing, deploying, applying, transplanting, implanting, direct seeding, directed migration, directed tracking, in setting, laminating, injection, absorption and combinations thereof.

### EXEMPLARY EMBODIMENTS

1. A composition comprising at least a portion of a mammalian material interface stimulated ex vivo or artificially comprising core potent cellular entities and supportive entities, wherein the composition is capable of assembling functional material.
2. The composition of any of the preceding claims, wherein the mammalian material interface is derived from a cutaneous tissue interface.
3. The composition of any of the preceding claims, wherein the cutaneous tissue interface comprises an epidermal-dermal interface.
4. The composition of any of the preceding claims, wherein the cutaneous tissue interface comprises a papillary-reticular dermal interface.
5. The composition of any of the preceding claims, wherein the cutaneous tissue interface comprises a dermal-hypodermal interface.
6. The composition of any of the preceding claims, wherein the cutaneous tissue interface comprises a hypodermal-subdermal interface.
7. The composition of any of the preceding claims, wherein the cutaneous tissue interface comprises an appendage-substrate interface.
8. The composition of any of the preceding claims wherein the mammalian material interface is derived from an osseous tissue interface.
9. The composition of any of the preceding claims, wherein the osseous tissue interface comprises a peri-cortical tissue interface.
10. The composition of any of the preceding claims, wherein the osseous tissue interface comprises a peri-lamellar tissue interface.
11. The composition of any of the preceding claims, wherein the osseous tissue interface comprises a peri-trabecular tissue interface.
12. The composition of any of the preceding claims, wherein the osseous tissue interface comprises a cortico-cancellous tissue interface.
13. The composition of any of the preceding claims, wherein the mammalian material interface is derived from a musculoskeletal tissue interface.
14. The composition of any of the preceding claims, wherein the musculoskeletal tissue interface comprises a myo-epimysial tissue interface.
15. The composition of any of the preceding claims, wherein the musculoskeletal tissue interface comprises a myo-perimysial tissue interface.
16. The composition of any of the preceding claims, wherein the musculoskeletal tissue interface comprises a myo-endomysial tissue interface.
17. The composition of any of the preceding claims, wherein the musculoskeletal tissue interface comprises a myo-fascial tissue interface.
18. The composition of any of the preceding claims, wherein the musculoskeletal tissue interface comprises a tendon-muscle tissue interface.
19. The composition of any of the preceding claims, wherein the musculoskeletal tissue interface comprises a tendon-bone tissue interface.
20. The composition of any of the preceding claims, wherein the musculoskeletal tissue interface comprises a ligament-bone tissue interface.
21. The composition of any of the preceding claims, wherein the mammalian material interface is derived from a smooth muscle tissue interface.
22. The composition of any of the preceding claims, wherein the smooth muscle tissue interface comprises a perivascular tissue interface.
23. The composition of any of the preceding claims, wherein the smooth muscle tissue interface comprises a perivisceral tissue interface.
24. The composition of any of the preceding claims, wherein the smooth muscle tissue interface comprises a perineural tissue interface.
25. The composition of any of the preceding claims, wherein the mammalian material interface is derived from a cardiac muscle tissue interface.
26. The composition of any of the preceding claims, wherein the cardiac muscle tissue interface comprises an endocardial-myocardial tissue interface.
27. The composition of any of the preceding claims, wherein the cardiac muscle tissue interface comprises a myocardial-epicardial tissue interface.
28. The composition of any of the preceding claims, wherein the cardiac muscle tissue interface comprises an epicardial-pericardial tissue interface.
29. The composition of any of the preceding claims, wherein the cardiac muscle tissue interface comprises a pericardial-adipose tissue interface.
30. The composition of any of the preceding claims, wherein the mammalian material interface is derived from a cartilage tissue interface.
31. The composition of any of the preceding claims, wherein the cartilage tissue interface comprises a chondrial-perichondrial tissue interface.
32. The composition of any of the preceding claims, wherein the cartilage tissue interface comprises a chondrial-endochondrial tissue interface.
33. The composition of any of the preceding claims, wherein the cartilage tissue interface comprises an endochondrial-subchondral bone interface.
34. The composition of any of the preceding claims, wherein the cartilage tissue interface comprises a chondrial-endochondrial bone interface.
35. The composition of any of the preceding claims, wherein the cartilage tissue interface comprises an endochondrial-subchondral bone interface.
36. The composition of any of the preceding claims, wherein the mammalian material interface is derived from an adipose tissue interface.
37. The composition of any of the preceding claims, wherein the adipose tissue interface comprises an adipo-perivascular tissue interface.
38. The composition of any of the preceding claims, wherein the adipose tissue interface comprises an adipo-peristromal tissue interface.
39. The composition of any of the preceding claims, wherein the mammalian material interface is derived from a gastrointestinal tissue interface.
40. The composition of any of the preceding claims, wherein the gastrointestinal tissue interface comprises a mucosal-submucosal tissue interface.
41. The composition of any of the preceding claims, wherein the gastrointestinal tissue interface comprises a sub-mucosal-muscularis tissue interface.
42. The composition of any of the preceding claims, wherein the gastrointestinal tissue interface comprises a muscularis-serosal tissue interface.
43. The composition of any of the preceding claims, wherein the gastrointestinal tissue interface comprises a serosal-mesentery tissue interface.
44. The composition of any of the preceding claims, wherein the gastrointestinal tissue interface comprises a myo-neural tissue interface.
45. The composition of any of the preceding claims, wherein the gastrointestinal tissue interface comprises a submucosal-neural tissue interface.
46. The composition of any of the preceding claims, wherein the mammalian material interface is derived from a pulmonary tissue interface.
47. The composition of any of the preceding claims, wherein the pulmonary tissue interface comprises a mucosal-submucosal tissue interface.
48. The composition of any of the preceding claims, wherein the pulmonary tissue interface comprises a sub-mucosal-muscularis tissue interface.
49. The composition of any of the preceding claims, wherein the pulmonary tissue interface comprises a sub-mucosal-cartilage tissue interface.
50. The composition of any of the preceding claims, wherein the pulmonary tissue interface comprises muscular-adventitial tissue interface.
51. The composition of any of the preceding claims, wherein the pulmonary tissue interface comprises a ductal-adventitial tissue interface.
52. The composition of any of the preceding claims, wherein the pulmonary tissue interface comprises a parenchymal-serosal tissue interface.
53. The composition of any of the preceding claims, wherein the pulmonary tissue interface comprises a serosal-mesentery tissue interface.
54. The composition of any of the preceding claims, wherein the pulmonary tissue interface comprises a myo-neural tissue interface.
55. The composition of any of the preceding claims, wherein the pulmonary tissue interface comprises a submucosal-neural tissue interface.
56. The composition of any of the preceding claims, wherein the mammalian material interface is derived from an esophageal tissue interface.
57. The composition of any of the preceding claims, wherein the esophageal tissue interface comprises a mucosal-submucosal tissue interface.
58. The composition of any of the preceding claims, wherein the esophageal tissue interface comprises a sub-mucosal-muscularis tissue interface.
59. The composition of any of the preceding claims, wherein the esophageal tissue interface comprises a muscularis-adventitial tissue interface.
60. The composition of any of the preceding claims, wherein the esophageal tissue interface comprises a myo-neural tissue interface.
61. The composition of any of the preceding claims, wherein the esophageal tissue interface comprises a submucosal-neural tissue interface.
62. The composition of any of the preceding claims, wherein the mammalian material interface is derived from a gastric tissue interface.
63. The composition of any of the preceding claims, wherein the gastric tissue interface comprises a mucosal-submucosal tissue interface.
64. The composition of any of the preceding claims, wherein the gastric tissue interface comprises a sub-mucosal-muscularis tissue interface.
65. The composition of any of the preceding claims, wherein the gastric tissue interface comprises a muscularis-serosal tissue interface.
66. The composition of any of the preceding claims, wherein the gastric tissue interface comprises a myo-neural tissue interface.
67. The composition of any of the preceding claims, wherein the gastric tissue interface comprises a submucosal-neural tissue interface.
68. The composition of any of the preceding claims, wherein the mammalian material interface is derived from a renal tissue interface.
69. The composition of any of the preceding claims, wherein the renal tissue interface comprises a capsule-cortical tissue interface.
70. The composition of any of the preceding claims, wherein the renal tissue interface comprises a cortical-medullary tissue interface.
71. The composition of any of the preceding claims, wherein the renal tissue interface comprises a neuro-parenchymal tissue interface.
72. The composition of any of the preceding claims, wherein the mammalian material interface is derived from a hepatic tissue interface.
73. The composition of any of the preceding claims, wherein the hepatic tissue interface comprises a ductal epithelial-parenchymal tissue interface.
74. The composition of any of the preceding claims, wherein the hepatic tissue interface comprises a capsular-parenchymal tissue interface.
75. The composition of any of the preceding claims, wherein the mammalian material interface is derived from a pancreatic tissue interface.
76. The composition of any of the preceding claims, wherein the pancreatic tissue interface comprises a ductal epithelial-parenchymal tissue interface.
77. The composition of any of the preceding claims, wherein the pancreatic tissue interface comprises a glandular epithelial-parenchymal tissue interface.
78. The composition of any of the preceding claims, wherein the mammalian material interface is derived from a blood vessel tissue interface.
79. The composition of any of the preceding claims, wherein the blood vessel tissue interface comprises an endothelial-tunica tissue interface.
80. The composition of any of the preceding claims, wherein the blood vessel tissue interface comprises a tunica-tunica tissue interface.
81. The composition of any of the preceding claims, wherein the mammalian material interface is derived from a lymphatic tissue interface.
82. The composition of any of the preceding claims, wherein the lymphatic tissue interface comprises a cortico-medullary tissue interface.
83. The composition of any of the preceding claims, wherein the lymphatic tissue interface comprises a medullary-capsule tissue interface.
84. The composition of any of the preceding claims, wherein the lymphatic tissue interface comprises a capsule-pulp tissue interface.
85. The composition of any of the preceding claims, wherein the mammalian material interface is derived from a central nervous tissue interface.
86. The composition of any of the preceding claims, wherein the central nervous tissue interface comprises a dural-cortex tissue interface.
87. The composition of any of the preceding claims, wherein the central nervous tissue interface comprises a cortical grey matter-medullary white matter tissue interface.
88. The composition of any of the preceding claims, wherein the central nervous tissue interface comprises a meningeal-neural tissue interface.
89. The composition of any of the preceding claims, wherein the mammalian material interface is derived from a urogenital tissue interface.
90. The composition of any of the preceding claims, wherein the urogenital tissue interface comprises an epithelial-mucosal tissue interface.
91. The composition of any of the preceding claims, wherein the urogenital tissue interface comprises a mucosal-muscular tissue interface.
92. The composition of any of the preceding claims, wherein the urogenital tissue interface comprises a muscular-adventitial tissue interface.
93. The composition of any of the preceding claims, wherein the urogenital tissue interface comprises a corporal-vascular tissue interface.
94. The composition of any of the preceding claims, wherein the urogenital tissue interface comprises a corporal-muscular tissue interface.
95. The composition of any of the preceding claims, wherein the mammalian material interface is derived from a glandular tissue interface.
96. The composition of any of the preceding claims, wherein the glandular tissue interface comprises an epithelial-parenchymal tissue interface.
97. The composition of any of the preceding claims, wherein the mammalian material interface is derived from a dental tissue interface.
98. The composition of any of the preceding claims, wherein the dental tissue interface comprises a dentin-pulp tissue interface.
99. The composition of any of the preceding claims, wherein the mammalian material interface is derived from a peripheral nerve tissue interface.
100. The composition of any of the preceding claims, wherein the peripheral nerve tissue interface comprises an epineural-perineural tissue interface.
101. The composition of any of the preceding claims, wherein the peripheral nerve tissue interface comprises a perineural-endoneural tissue interface.
102. The composition of any of the preceding claims, wherein the peripheral nerve tissue interface comprises an endoneural-axonal.
103. The composition of any of the preceding claims, wherein the mammalian material interface is derived from a birth tissue interface.
104. The composition of any of the preceding claims, wherein the birth tissue interface comprises an amnion-fluid tissue interface.
105. The composition of any of the preceding claims, wherein the birth tissue interface comprises an epithelial-sub-epithelial tissue interface.
106. The composition of any of the preceding claims, wherein the birth tissue interface comprises an epithelial-stroma tissue interface.
107. The composition of any of the preceding claims, wherein the birth tissue interface comprises a compact-fibroblast tissue interface.
108. The composition of any of the preceding claims, wherein the birth tissue interface comprises a fibroblast-intermediate tissue interface.
109. The composition of any of the preceding claims, wherein the birth tissue interface comprises an intermediate-reticular tissue interface.
110. The composition of any of the preceding claims, wherein the birth tissue interface comprises an amnio-chroion tissue interface.
111. The composition of any of the preceding claims, wherein the birth tissue interface comprises a reticular-trophoblast tissue interface.
112. The composition of any of the preceding claims, wherein the birth tissue interface comprises a trophoblast-uterine tissue interface.
113. The composition of any of the preceding claims, wherein the birth tissue interface comprises a trophoblast-decidua tissue interface.
114. The composition of any of the preceding claims, wherein the mammalian material interface is derived from an optic tissue interface.
115. The composition of any of the preceding claims, wherein the optic tissue interface comprises an epithelial-membrane tissue interface.
116. The composition of any of the preceding claims, wherein the optic tissue interface comprises a membrane-stroma tissue interface.
117. The composition of any of the preceding claims, wherein the optic tissue interface comprises a stromal-membrane tissue interface.
118. The composition of any of the preceding claims, wherein the optic tissue interface comprises a membrane-endothelial tissue interface.
119. The composition of any of the preceding claims, wherein the optic tissue interface comprises an endothelial-fluid tissue interface.
120. The composition of any of the preceding claims, wherein the optic tissue interface comprises a scleral-choroid tissue interface.
121. The composition of any of the preceding claims, wherein the optic tissue interface comprises a choroid-epithelial tissue interface.
122. The composition of any of the preceding claims, wherein the optic tissue interface comprises an epithelial-segmental photoreceptor tissue interface.
123. The composition of any of the preceding claims, wherein the optic tissue interface comprises a segmental photoreceptor-membrane tissue interface.
124. The composition of any of the preceding claims, wherein the optic tissue interface comprises a membrane-outer nuclear layer tissue interface.
125. The composition of any of the preceding claims, wherein the optic tissue interface comprises an outer nuclear layer-outer plexiform tissue interface.
126. The composition of any of the preceding claims, wherein the optic tissue interface comprises an outer plexiform-inner plexiform tissue interface.
127. The composition of any of the preceding claims, wherein the optic tissue interface comprises an inner plexiform-ganglion tissue interface.
128. The composition of any of the preceding claims, wherein the optic tissue interface comprises a ganglion-neural fiber tissue interface.
129. The composition of any of the preceding claims, wherein the optic tissue interface comprises a neural fiber-membrane tissue interface.
130. The composition of any of the preceding claims, wherein the optic tissue interface comprises a membrane-fluid tissue interface.
131. The composition of any of the preceding claims, wherein the supportive entities comprise mesenchymal derived cellular populations.
132. The composition of any of the preceding claims, wherein the supportive entities comprise cellular populations, extracellular matrix elements, or combinations thereof.
133. The composition of any of the preceding claims, further comprising a delivery substrate.
134. The composition of any of the preceding claims, wherein the delivery substrate is selected from a scaffold, matrix, particle, cells, fiber, or combinations thereof.
135. The composition of any of the preceding claims, further comprising a supplement selected from a growth factor, an analyte, a LGR interactive element, or combinations thereof
136. The composition of any of the preceding claims, wherein the analyte is selected from a migratory analyte, a recruiting analyte, a stimulatory agent, an inhibitory agent, or combinations thereof.
137. A method of producing a composition, comprising:
   isolating at least a portion of a mammalian material interface comprising core potent cellular entities and supportive entities; and
   developing a reactive and stimulated interface to provide the composition, wherein the composition is capable of assembling functional material.
138. The method of any of the preceding claims, wherein the mammalian material interface is derived from a cutaneous tissue interface.
139. The method of any of the preceding claims, wherein the cutaneous tissue interface comprises an epidermal-dermal interface.
140. The method of any of the preceding claims, wherein the cutaneous tissue interface comprises a papillary-reticular dermal interface.
141. The method of any of the preceding claims, wherein the cutaneous tissue interface comprises a dermal-hypodermal interface.
142. The method of any of the preceding claims, wherein the cutaneous tissue interface comprises a hypodermal-subdermal interface.
143. The method of any of the preceding claims, wherein the cutaneous tissue interface comprises an appendage-substrate interface.
144. The method of any of the preceding claims, wherein the mammalian material interface is derived from an osseous tissue interface.
145. The method of any of the preceding claims, wherein the osseous tissue interface comprises a peri-cortical tissue interface.
146. The method of any of the preceding claims, wherein the osseous tissue interface comprises a peri-lamellar tissue interface.
147. The method of any of the preceding claims, wherein the osseous tissue interface comprises a peri-trabecular tissue interface.
148. The method of any of the preceding claims, wherein the osseous tissue interface comprises a cortico-cancellous tissue interface.
149. The method of any of the preceding claims, wherein the mammalian material interface is derived from a musculoskeletal tissue interface.
150. The method of any of the preceding claims, wherein the musculoskeletal tissue interface comprises a myo-epimysial tissue interface.
151. The method of any of the preceding claims, wherein the musculoskeletal tissue interface comprises a myo-perimysial tissue interface.
152. The method of any of the preceding claims, wherein the musculoskeletal tissue interface comprises a myo-endomysial tissue interface.
153. The method of any of the preceding claims, wherein the musculoskeletal tissue interface comprises a myo-fascial tissue interface.
154. The method of any of the preceding claims, wherein the musculoskeletal tissue interface comprises a tendon-muscle tissue interface.
155. The method of any of the preceding claims, wherein the musculoskeletal tissue interface comprises a tendon-bone tissue interface.
156. The method of any of the preceding claims, wherein the musculoskeletal tissue interface comprises a ligament-bone tissue interface.
157. The method of any of the preceding claims, wherein the mammalian material interface is derived from a smooth muscle tissue interface.
158. The method of any of the preceding claims, wherein the smooth muscle tissue interface comprises a perivascular tissue interface.
159. The method of any of the preceding claims, wherein the smooth muscle tissue interface comprises a perivisceral tissue interface.
160. The method of any of the preceding claims, wherein the smooth muscle tissue interface comprises a perineural tissue interface.
161. The method of any of the preceding claims, wherein the mammalian material interface is derived from a cardiac muscle tissue interface.
162. The method of any of the preceding claims, wherein the cardiac muscle tissue interface comprises an endocardial-myocardial tissue interface.
163. The method of any of the preceding claims, wherein the cardiac muscle tissue interface comprises a myocardial-epicardial tissue interface.
164. The method of any of the preceding claims, wherein the cardiac muscle tissue interface comprises an epicardial-pericardial tissue interface.
165. The method of any of the preceding claims, wherein the cardiac muscle tissue interface comprises a pericardial-adipose tissue interface.
166. The method of any of the preceding claims, wherein the mammalian material interface is derived from a cartilage tissue interface.
167. The method of any of the preceding claims, wherein the cartilage tissue interface comprises a chondrial-perichondrial tissue interface.
168. The method of any of the preceding claims, wherein the cartilage tissue interface comprises a chondrial-endochondrial tissue interface.
169. The method of any of the preceding claims, wherein the cartilage tissue interface comprises an endochondrial-subchondral bone interface.
170. The method of any of the preceding claims, wherein the cartilage tissue interface comprises a chondrial-endochondrial bone interface.
171. The method of any of the preceding claims, wherein the cartilage tissue interface comprises an endochondrial-subchondral bone interface.
172. The method of any of the preceding claims, wherein the mammalian material interface is derived from an adipose tissue interface.
173. The method of any of the preceding claims, wherein the adipose tissue interface comprises an adipo-perivascular tissue interface.
174. The method of any of the preceding claims, wherein the adipose tissue interface comprises an adipo-peristromal tissue interface.
175. The method of any of the preceding claims, wherein the mammalian material interface is derived from a gastrointestinal tissue interface.
176. The method of any of the preceding claims, wherein the gastrointestinal tissue interface comprises a mucosal-submucosal tissue interface.
177. The method of any of the preceding claims, wherein the gastrointestinal tissue interface comprises a sub-mucosal-muscularis tissue interface.
178. The method of any of the preceding claims, wherein the gastrointestinal tissue interface comprises a muscularis-serosal tissue interface.
179. The method of any of the preceding claims, wherein the gastrointestinal tissue interface comprises a serosal-mesentery tissue interface.
180. The method of any of the preceding claims, wherein the gastrointestinal tissue interface comprises a myo-neural tissue interface.
181. The method of any of the preceding claims, wherein the gastrointestinal tissue interface comprises a submucosal-neural tissue interface.
182. The method of any of the preceding claims, wherein the mammalian material interface is derived from a pulmonary tissue interface.
183. The method of any of the preceding claims, wherein the pulmonary tissue interface comprises a mucosal-submucosal tissue interface.
184. The method of any of the preceding claims, wherein the pulmonary tissue interface comprises a sub-mucosal-muscularis tissue interface.
185. The method of any of the preceding claims, wherein the pulmonary tissue interface comprises a sub-mucosal-cartilage tissue interface.
186. The method of any of the preceding claims, wherein the pulmonary tissue interface comprises muscular-adventitial tissue interface.
187. The method of any of the preceding claims, wherein the pulmonary tissue interface comprises a ductal-adventitial tissue interface.
188. The method of any of the preceding claims, wherein the pulmonary tissue interface comprises a parenchymal-serosal tissue interface.
189. The method of any of the preceding claims, wherein the pulmonary tissue interface comprises a serosal-mesentery tissue interface.
190. The method of any of the preceding claims, wherein the pulmonary tissue interface comprises a myo-neural tissue interface.
191. The method of any of the preceding claims, wherein the pulmonary tissue interface comprises a submucosal-neural tissue interface.
192. The method of any of the preceding claims, wherein the mammalian material interface is derived from an esophageal tissue interface.
193. The method of any of the preceding claims, wherein the esophageal tissue interface comprises a mucosal-submucosal tissue interface.
194. The method of any of the preceding claims, wherein the esophageal tissue interface comprises a sub-mucosal-muscularis tissue interface.
195. The method of any of the preceding claims, wherein the esophageal tissue interface comprises a muscularis-adventitial tissue interface.
196. The method of any of the preceding claims, wherein the esophageal tissue interface comprises a myo-neural tissue interface.
197. The method of any of the preceding claims, wherein the esophageal tissue interface comprises a submucosal-neural tissue interface.
198. The method of any of the preceding claims, wherein the mammalian material interface is derived from a gastric tissue interface.
199. The method of any of the preceding claims, wherein the gastric tissue interface comprises a mucosal-submucosal tissue interface.
200. The method of any of the preceding claims, wherein the gastric tissue interface comprises a sub-mucosal-muscularis tissue interface.
201. The method of any of the preceding claims, wherein the gastric tissue interface comprises a muscularis-serosal tissue interface.
202. The method of any of the preceding claims, wherein the gastric tissue interface comprises a myo-neural tissue interface.
203. The method of any of the preceding claims, wherein the gastric tissue interface comprises a submucosal-neural tissue interface.
204. The method of any of the preceding claims, wherein the mammalian material interface is derived from a renal tissue interface.
205. The method of any of the preceding claims, wherein the renal tissue interface comprises a capsule-cortical tissue interface.
206. The method of any of the preceding claims, wherein the renal tissue interface comprises a cortical-medullary tissue interface.
207. The method of any of the preceding claims, wherein the renal tissue interface comprises a neuro-parenchymal tissue interface.
208. The method of any of the preceding claims, wherein the mammalian material interface is derived from a hepatic tissue interface.
209. The method of any of the preceding claims, wherein the hepatic tissue interface comprises a ductal epithelial-parenchymal tissue interface.
210. The method of any of the preceding claims, wherein the hepatic tissue interface comprises a capsular-parenchymal tissue interface.
211. The method of any of the preceding claims, wherein the mammalian material interface is derived from a pancreatic tissue interface.
212. The method of any of the preceding claims, wherein the pancreatic tissue interface comprises a ductal epithelial-parenchymal tissue interface.
213. The method of any of the preceding claims, wherein the pancreatic tissue interface comprises a glandular epithelial-parenchymal tissue interface.
214. The method of any of the preceding claims, wherein the mammalian material interface is derived from a blood vessel tissue interface.
215. The method of any of the preceding claims, wherein the blood vessel tissue interface comprises an endothelial-tunica tissue interface.
216. The method of any of the preceding claims, wherein the blood vessel tissue interface comprises a tunica-tunica tissue interface.
217. The method of any of the preceding claims, wherein the mammalian material interface is derived from a lymphatic tissue interface.
218. The method of any of the preceding claims, wherein the lymphatic tissue interface comprises a cortico-medullary tissue interface.
219. The method of any of the preceding claims, wherein the lymphatic tissue interface comprises a medullary-capsule tissue interface.
220. The method of any of the preceding claims, wherein the lymphatic tissue interface comprises a capsule-pulp tissue interface.
221. The method of any of the preceding claims, wherein the mammalian material interface is derived from a central nervous tissue interface.
222. The method of any of the preceding claims, wherein the central nervous tissue interface comprises a dural-cortex tissue interface.
223. The method of any of the preceding claims, wherein the central nervous tissue interface comprises a cortical grey matter-medullary white matter tissue interface.
224. The method of any of the preceding claims, wherein the central nervous tissue interface comprises a meningeal-neural tissue interface.
225. The method of any of the preceding claims, wherein the mammalian material interface is derived from a urogenital tissue interface.
226. The method of any of the preceding claims, wherein the urogenital tissue interface comprises an epithelial-mucosal tissue interface.
227. The method of any of the preceding claims, wherein the urogenital tissue interface comprises a mucosal-muscular tissue interface.
228. The method of any of the preceding claims, wherein the urogenital tissue interface comprises a muscular-adventitial tissue interface.
229. The method of any of the preceding claims, wherein the urogenital tissue interface comprises a corporal-vascular tissue interface.
230. The method of any of the preceding claims, wherein the urogenital tissue interface comprises a corporal-muscular tissue interface.
231. The method of any of the preceding claims, wherein the mammalian material interface is derived from a glandular tissue interface.
232. The method of any of the preceding claims, wherein the glandular tissue interface comprises an epithelial-parenchymal tissue interface.
233. The method of any of the preceding claims, wherein the mammalian material interface is derived from a dental tissue interface.
234. The method of any of the preceding claims, wherein the dental tissue interface comprises a dentin-pulp tissue interface.
235. The method of any of the preceding claims, wherein the mammalian material interface is derived from a peripheral nerve tissue interface.
236. The method of any of the preceding claims, wherein the peripheral nerve tissue interface comprises an epineural-perineural tissue interface.
237. The method of any of the preceding claims, wherein the peripheral nerve tissue interface comprises a perineural-endoneural tissue interface.
238. The method of any of the preceding claims, wherein the peripheral nerve tissue interface comprises an endoneural-axonal.
239. The method of any of the preceding claims, wherein the mammalian material interface is derived from a birth tissue interface.
240. The method of any of the preceding claims, wherein the birth tissue interface comprises an amnion-fluid tissue interface.
241. The method of any of the preceding claims, wherein the birth tissue interface comprises an epithelial-sub-epithelial tissue interface.
242. The method of any of the preceding claims, wherein the birth tissue interface comprises an epithelial-stroma tissue interface.
243. The method of any of the preceding claims, wherein the birth tissue interface comprises a compact-fibroblast tissue interface.
244. The method of any of the preceding claims, wherein the birth tissue interface comprises a fibroblast-intermediate tissue interface.
245. The method of any of the preceding claims, wherein the birth tissue interface comprises an intermediate-reticular tissue interface.
246. The method of any of the preceding claims, wherein the birth tissue interface comprises an amnio-chroion tissue interface.
247. The method of any of the preceding claims, wherein the birth tissue interface comprises a reticular-trophoblast tissue interface.
248. The method of any of the preceding claims, wherein the birth tissue interface comprises a trophoblast-uterine tissue interface.
249. The method of any of the preceding claims, wherein the birth tissue interface comprises a trophoblast-decidua tissue interface.
250. The method of any of the preceding claims, wherein the mammalian material interface is derived from an optic tissue interface.
251. The method of any of the preceding claims, wherein the optic tissue interface comprises an epithelial-membrane tissue interface.
252. The method of any of the preceding claims, wherein the optic tissue interface comprises a membrane-stroma tissue interface.
253. The method of any of the preceding claims, wherein the optic tissue interface comprises a stromal-membrane tissue interface.
254. The method of any of the preceding claims, wherein the optic tissue interface comprises a membrane-endothelial tissue interface.
255. The method of any of the preceding claims, wherein the optic tissue interface comprises an endothelial-fluid tissue interface.
256. The method of any of the preceding claims, wherein the optic tissue interface comprises a scleral-choroid tissue interface.
257. The method of any of the preceding claims, wherein the optic tissue interface comprises a choroid-epithelial tissue interface.
258. The method of any of the preceding claims, wherein the optic tissue interface comprises an epithelial-segmental photoreceptor tissue interface.
259. The method of any of the preceding claims, wherein the optic tissue interface comprises a segmental photoreceptor-membrane tissue interface.
260. The method of any of the preceding claims, wherein the optic tissue interface comprises a membrane-outer nuclear layer tissue interface.
261. The method of any of the preceding claims, wherein the optic tissue interface comprises an outer nuclear layer-outer plexiform tissue interface.
262. The method of any of the preceding claims, wherein the optic tissue interface comprises an outer plexiform-inner plexiform tissue interface.
263. The method of any of the preceding claims, wherein the optic tissue interface comprises an inner plexiform-ganglion tissue interface.
264. The method of any of the preceding claims, wherein the optic tissue interface comprises a ganglion-neural fiber tissue interface.
265. The method of any of the preceding claims, wherein the optic tissue interface comprises a neural fiber-membrane tissue interface.
266. The method of any of the preceding claims, wherein the optic tissue interface comprises a membrane-fluid tissue interface.
267. The method of any of the preceding claims, wherein the supportive entities comprise mesenchymal derived cellular populations.
268. The method of any of the preceding claims, wherein the supportive entities are selected from cellular populations, extracellular matrix elements, or combinations thereof.
269. The method of any of the preceding claims, further comprising adding a supplement selected from a growth factor, an analyte, a LGR interactive element, or combinations thereof
270. The method of any of the preceding claims, wherein the analyte is selected from of a migratory analyte, a recruiting analyte, a stimulatory agent, an inhibitory agent, or combinations thereof.
271. The method of any of the preceding claims, further comprising adding the composition to a delivery substrate.
272. The method of any of the preceding claims, wherein the delivery substrate is selected from a scaffold, matrix, particle, cells, fiber, or combinations thereof 273. The method of any of the preceding claims, further comprising cryopreserving the composition.
274. The method of any of the preceding claims, further comprising lyophilizing the composition.
275. A composition produced by the method of any of the preceding claims.
276. A method of treating a disease or disorder of tissue, comprising administering a composition to a target site of a subject in need thereof, wherein
   the disease or disorder of the tissue results in:
      (i) loss or destruction of the tissue;
      (ii) failure of formation of the tissue; or
      (iii) formation of abnormal tissue; and
   the composition comprises at least a portion of a mammalian material interface comprising core potent cellular entities and supportive entities, wherein the composition is capable of assembling functional tissue.

### EXAMPLES

### Example 1

Starting with a mammalian specimen material, place the mammalian specimen material in a series of one or more washes using isotonic, biocompatible solution (e.g. 0.9% NaCl, HBSS, PBS, DMEM, RPMI, lactated ringers, 5% dextrose in water, 3.2% sodium citrate) (with or without antimicrobial agent(s)) for approximately 5 minutes each with gentle agitation, rocking, shaking, and/or stirring.

Once washed, locate a tissue interface. Methods of location, including the use of equipment and/or supportive systems, are well known in the art and may be used to locate the appropriate tissue interface(s). If the complete interface is not present, locate the area where a sub-compartment or sub-set of the interface (i.e., sub-interface) is present.

Separate the interface either in complete or sub-compartment (i.e., sub-interface) from the remainder of the mammalian specimen material (i.e., the non-interface materials). Continue such action of separating the interface until sufficient material for the application at hand, for example, volume/mass of material needed to treat the size of the wound, is obtained. Methods of separation, including the use of equipment and/or supportive systems, are well known in the art and may be used to separate the appropriate interface(s).

Place the complete interface or sub-interface materials into a solution of supportive media solution (e.g., HBSS, PBS) and add an effective reactive stimulant and/or a related accelerator adjuvant (e.g., collagenase, testicular hyaluronidase, trypsin) for 1-15 minutes in a temperature controlled CO₂ environment. Methods of reactive stimulation, including the use of reagents, equipment and/or supportive systems, are well known in the art and may be used to provide the reactive and stimulated interface.

Terminate the action of reactive stimulant and/or related accelerator adjuvant with the appropriate termination agent, solution, factor and/or media (e.g., EDTA). Methods of termination, including the use of reagents, equipment and/or supportive systems, are well known in the art and may be used to terminate such action(s).

Collect the stimulated interface from solution. Keep the solution. Methods of collection, including the use of reagents, equipment and/or supportive systems, are well known in the art and may be used to collect the reactive and stimulated interface.

Place the collected reactive and stimulated interface into a temporary sterile vessel with small amount of an isotonic biocompatible solution and store. Return to the remaining non-interfaced materials (i.e., located within the washed mammalian specimen).

Place the non-interface materials into a solution of supportive media solution and add effective reactive stimulant and/or related accelerator adjuvant for 1-15 minutes in a temperature controlled CO₂ environment. Methods of reactive stimulation, including the use of reagents, equipment and/or supportive systems, are well known in the art and may be used to provide reactive and stimulated non-interface material.

Terminate the action of reactive stimulant and/or related accelerator adjuvant with the appropriate termination agent, solution, factor and/or media. Methods of termination, including the use of reagents, equipment and/or supportive systems, are well known in the art and may be used to terminate such action(s).

Collect the reactive and stimulated non-interface materials from solution. Keep the solution for later use. Methods of collection, including the use of reagents, equipment and/or supportive systems, are well known in the art and may be used to collect the reactive and stimulated non-interface material.

Add the reactive and stimulated non-interface material to either a secondary culture vessel, ex-vivo support system, or bioreactor, add supplemental media materials and incubate in closed system which has the ability for environmental control and environmental alteration (e.g., incubator or bioreactor).

Add the reactive and stimulated interface material and the resultant processing fluid to either a secondary culture vessel, ex-vivo support system, or bioreactor and add supplemental media materials and incubate in closed system which has the ability for environmental control and environmental alteration (e.g., incubator or bioreactor).

Maintain ex-vivo support and/or culture of the processed material either separately or in a form of dual culture system(s) if desired or intended.

When needed deploy, place, or combine such reactive and stimulated materials in combination or separately to the target of interest.

### Example 2

An osseous tissue specimen was obtained and placed in a series of sequential washes using an isotonic, biocompatible solution (e.g. 0.9% NaCl, HBSS, PBS, DMEM, RPMI, lactated ringers, 5% dextrose in water, 3.2% sodium citrate) (with or without an antimicrobial agent) for approximately 5 minutes each with gentle agitation, rocking, shaking, and/or stirring.

Once washed, an osseous tissue interface was located and a sufficient amount of the osseous tissue interface material was separated from the remainder of the osseous tissue specimen (i.e., the non-interface materials).

The osseous tissue interface material was placed into a supportive media solution and an effective reactive stimulant and related accelerator adjuvant (e.g., collagenase, testicular hyaluronidase, trypsin) were added. Reactive stimulation occurred for 1-15 minutes in a temperature controlled CO₂ environment and provided a reactive and stimulated osseous tissue interface.

The action of the reactive stimulant and related accelerator adjuvant was terminated with a termination agent (e.g., EDTA).

The reactive and stimulated osseous tissue interface was collected from solution. The solution was kept for later use.

The collected reactive and stimulated osseous tissue interface was placed into a temporary sterile vessel with small amount of isotonic biocompatible solution and stored to prevent desiccation of the collected reactive and stimulated osseous tissue interface.

The non-interface materials were placed into a supportive media solution (e.g., HBSS, PBS) and an effective reactive stimulant and related accelerator adjuvant were added. Reactive stimulation occurred for 1-15 minutes in a temperature controlled CO₂ environment and provided reactive and stimulated non-interface materials.

The action of the reactive stimulant and related accelerator adjuvant was terminated with a termination agent.

The reactive and stimulated non-interface materials were collected from solution. The solution was kept for later use.

The reactive and stimulated non-interface materials were added to an incubator, supplemental media materials were added, and the combination was incubated in a closed, environmentally controlled system.

The reactive and stimulated osseous tissue interface and the associated solution were added to an incubator, supplemental media materials were added, and the combination was incubated in closed, environmentally controlled system.

In separate instances, the reactive and stimulated osseous tissue interface and the combination of the reactive and stimulated osseous tissue interface and the reactive and stimulated non-interface materials were placed on targets of interest.

### Example 3

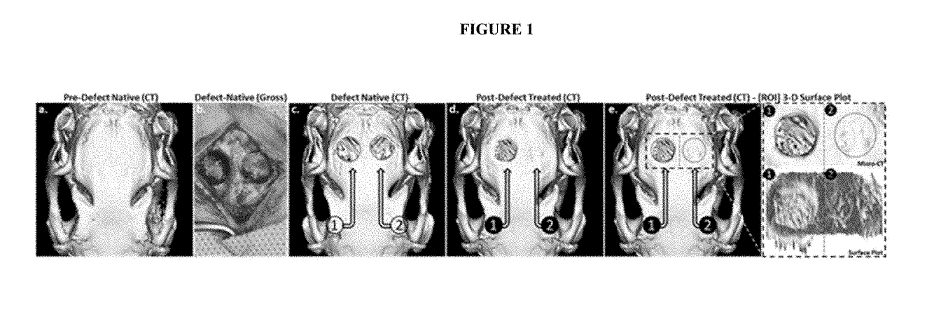

FIGS. 1a-e show Comparative Imaging of an osseous-derived composition as disclosed herein in Critical Sized Cranial Defect Model System. (a.) Three dimensional (3-D) micro computed tomography (micro-CT) native cranial bone displaying pre-defect left parietal and right parietal bones of in vivo model system at time point T^{PDN}. (b.) Gross image of surgically-created, complete, bi-parietal critical sized defects of both the left and right parietal bones within the in vivo model system at time point T⁰. (c.) 3-D micro-CT of surgically-created, complete (full-thickness), bi-parietal critical sized defects of both the left and right parietal bones within the in vivo model system. \textcircled{1} Indicates the right parietal bone region with 8 mm diameter defect at time point T⁰ which was un-treated and maintained as the defect control throughout study. \textcircled{2} Indicates the left parietal bone region with 8 mm defect which was treated with the osseous-derived composition and maintained as the defect-treated control throughout the study. (d.) 3-D micro-CT of surgically-created, complete, bi-parietal critical sized defects of both the left and right parietal bones within the in vivo model system at 4 weeks post-procedure and intervention (time point T^{PPI-4WK}). [symbol] Indicates the un-treated right parietal bone region (defect control) at 4 weeks. [symbol] Indicates the treated left parietal bone region (osseous-derived composition treatment) at 4 weeks. (e.) Depicts the relative margins of the primary bi-parietal defects (dotted circles) at time point T⁰; ROI (broken line box) indicates zoomed comparison of 4 weeks post-treatment defects of 3-D micro-CT and correlative 3-D thermal spectrum colored surface plot indicating relative surface depth and volumetric contour. Abbreviations: Pre-defect Native Timepoint (T^{PDN}): time point at which native skull was imaged prior to creation of defect; Defect Native Timepoint (T⁰): time point at which complete (full-thickness) 8 mm critically sized defects were created in parietal skull regions; Post-procedure and intervention at 4 weeks time point (T^{PPI-4WK}): time point at which 4 weeks have passed since the defects were created +/− treated with intervention. Accordingly, these results demonstrate that the osseous-derived compositions as disclosed herein are useful in methods for promoting bone regeneration.

### Example 4

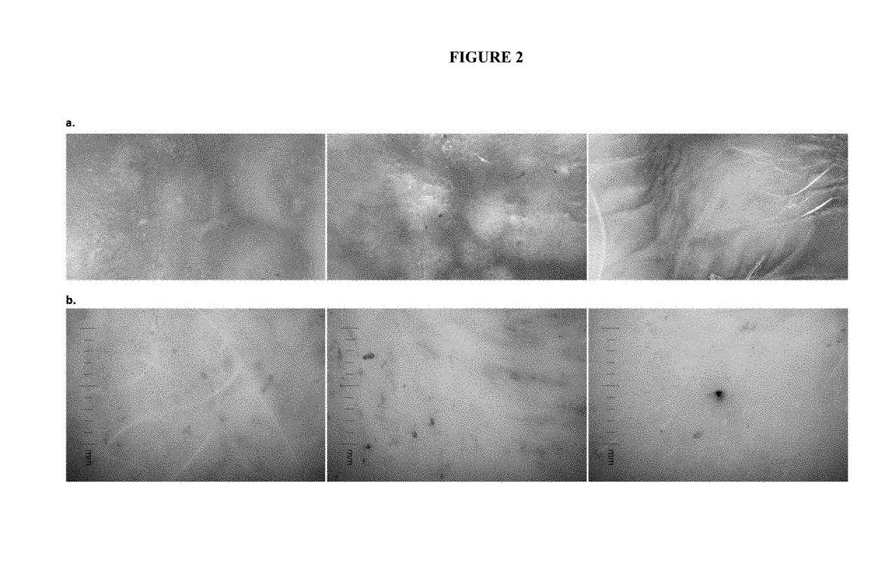

FIGS. 2a and 2b show progression of development of functional polarized tissue by a cutaneous-derived composition in a Cutaneous Model System (pig). FIGS. 2a and 2b show results on the same animal with different imaging platforms. The imaging platform of FIG. 2a was a high definition DSLR camera. The imaging platform of FIG. 2b was a polarized camera under magnification. (a.) Row—Depicts progression of the cutaneous-derived composition following placement into cutaneous void and the development of functionally-polarized full-thickness cutaneous tissue foci. (b.) Row—Depicts progression of the cutaneous-derived composition foci converge with propagating cutaneous-derived composition and/or with system which received said cutaneous-derived composition resulting in progressive generation of functionally-polarized, full-thickness cutaneous tissue throughout void.

### Example 5: Rabbit Long Bone Study

The long bone defect model consisted of 30 New Zealand White rabbits. A dorsal midline incision of 3-4 cm length was created over the forelimb in the approximate center of the diaphysis. Soft tissue between the extensor and flexor tendons was incised and the muscle elevated with care from the surface of the ulna for approximately 12-18 mm. An oscillating saw was used to cut the ulnar diaphysis. Care was taken to use crystalloid irrigation during the cutting procedure to prevent thermal injury to adjacent tissues. Care was utilized to ensure that the neighboring radial surface was not scored or nicked during the performance of the ostectomy procedure. After the proximal ostectomy cut was completed, the distal cut was completed, and the bone fragment was gently removed with minimal trauma to the intra-osseous ligament. Total ulnar defect size was 10 mm.

The defects were subjected to various treatments including treatment by an osseous-derived composition (e.g., AHBC) or left untreated. Table 1 shows the treatment groups:

TABLE 1

| Group # | N | Recipient | Treatment |
|---|---|---|---|
| 1 | 5 | New Zealand White | Untreated Defect |
| 2 | 5 | New Zealand White | DBM + BMP2 |
| 3 | 5 | New Zealand White | AHBC |

After removal of the bone from the defect site, it was placed into sterile transport media and processed on-site into an osseous-derived composition (e.g., AHBC). Processing was performed on an osseous tissue interface to create a stimulated composition comprising an aggregate of living core potent cellular entities and supportive entities where the living core potent cellular entities express a sequence of LGR4, LGR5, and/or LGR6. The AHBC was implanted into the defect and the muscle/soft tissue over the operative site was closed with absorbable suture. The subcutaneous and skin layers were closed with nonabsorbable suture in a layered fashion.

DBM+BMP-2 was prepared by combining (Human) DBM with 10 ug/mL of Bone Morphogenic Protein-2 (BMP-2). Defects were filled with DBM+BMP-2 using an equivocal volume as the amount of AHBC used for AHBC treated animals.

At the end of the study, tissues harvested included en-bloc forelimb. Downstream dissection of tissues included removal of overlying skin muscle and periosteum.

Imaging Methods:

Gross Imaging: DSLR photographs were acquired intra operative with a Canon 5DSR. Ex vivo images documented using the same setup with camera mounted on copy stand.

Vimago CT: The animals were scanned every two weeks during the eight-week study using the Vimago CT with the following settings:

60 mA

80 kV

7 ms

Time—32 seconds

Resolution—200 um

Micro-CT (μCT): A Quantum GX2, PerkinElmer instrument was used to image all ex vivo rabbit long bone specimens. Each specimen was imaged at 90 kV, 40 μA, FOV 36 mm, voxel size 90 μm, Al 0.5 CU 1.0 filter for 4 minutes to achieve best resolution. The images analyzed with Analyze software version 12.0 (AnalyzeDirect, Overland Park, Kans., USA).

Compound microscopy: Using the Leica 205 FA Equipped with a DFC7000T camera, each sample is imaged around its circumference using a time-lapse series to acquire a 360 view of each defect. Before imaging these samples, the radius is removed from the regrown ulna to show the best possible representation of the defect and regrowth region. In the untreated group, there is very little regrowth and therefore the radius is kept with the ulna. This is used to show a color image of the regrowth of bone and other tissue around and inside the defect region.

Scanning Electron Microscopy Imaging: Using the Zeiss Evo LS 10 environmental scanning electron microscope, images were taken of all long bone samples from each group to help determine viability of bone regeneration.

Second Harmonic Generation (SHG) Imaging: Second harmonic generation imaging was performed using a Leica SP8 multiphoton confocal microscope equipped with a Chameleon tunable two photon laser tuned to 880 nm using a 10×0.40 NA objective.

Raman Spectroscopy:

A confocal Raman microscope (Thermo Fisher Raman DXR) with a 10× objective and a laser wavelength of 785 nm (28 mW laser power) was used to collect spectra. A 25-um slit aperture was used to collect a spectral range between wavenumbers 500-3500 cm⁻¹. The estimated resolution was 2.3-4.3 cm⁻¹. Spectral data was collected using an exposure of 1 s with a signal to noise ratio of 300 to ensure the collected spectra represent the bulk material. For surface point scans, a total of 2-5 spectra were collected from arbitrary positions across the top surface of the defect. For surface line scans, 6 spectra were collected with 200 urn spacing between each point of collection.

Raman spectroscopy analysis was performed using OMNIC (Thermo Scientific) software for Dispersive Raman. Features available on OMNIC software were used to remove background fluorescence from all surface point scan spectra using 6^{th} order polynomial baseline fitting. Surface point spectra collected from each specimen were normalized and averaged to represent an individual animal. Overall group averages were calculated using average spectra from each individual animal within the group. OMEN IC Chemigrams for cross sectional area scans were created using ranges 950-965 cm⁻¹ for hydroxyapatite.

Gene Expression Methods:

Sample Collection: Tissue was collected from treated and untreated wounds and native ulnae following gross imaging. Tissue was collected in AllProtect (Qiagen), held at 4 C for 24 hr, and then moved to −80 C for storage until RNA extraction was performed.

RNA Extraction: Lysis of tissue was performed with PowerLyzer (Qiagen) for two cycles of 45 seconds at 3500 rpm with a 30 second dwell time between cycles. RNA was purified from the resulting tissue lysate using RNeasy Plus Universal Mini Kit (Qiagen). RNA was quantified using Nanodrop Lite (ThermoFisher Scientific).

Reverse Transcription and qRT-PCR: 800 ng of RNA was reverse transcribed to cDNA using RT2 First Strand Kit (Qiagen). Resulting cDNA was used as the template for RT2 PCR Profiler plates which were run according to manufacturer instructions (Qiagen) on a QuantStudio 12K Flex or QuantStudio 3 (Applied Biosystems, ThermoFisher Scientific). Data from these runs was analyzed comparing healed wounds to native tissue, and healed wounds to untreated controls. qPCR data was analyzed by the online Qiagen Data Analysis Center using the delta-delta Ct method to determine fold-regulation of individual genes and student's t-test (two-tail distribution and equal variances between the two samples) to determine significance.

Results:

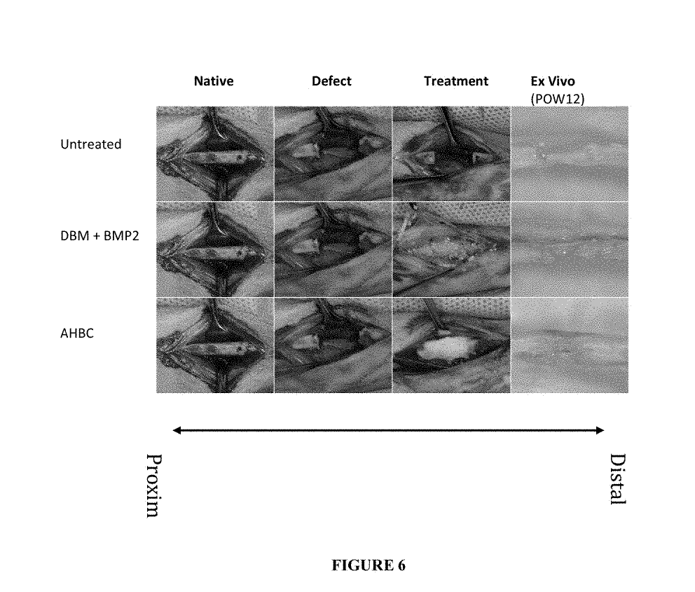

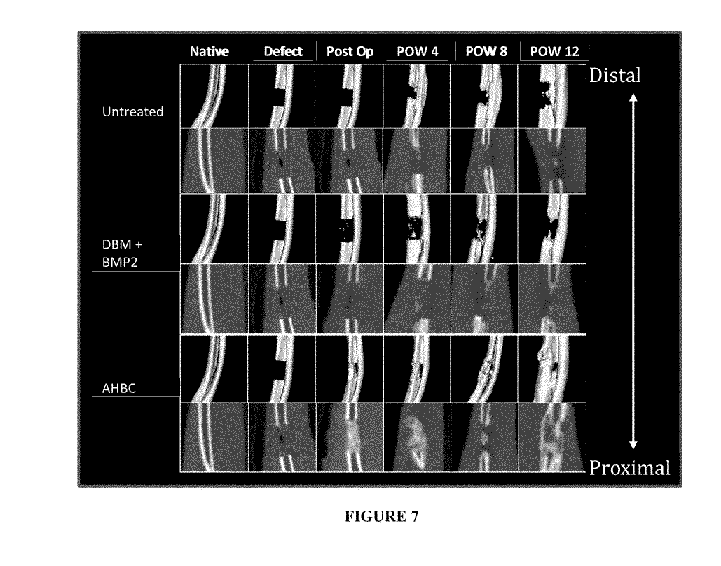

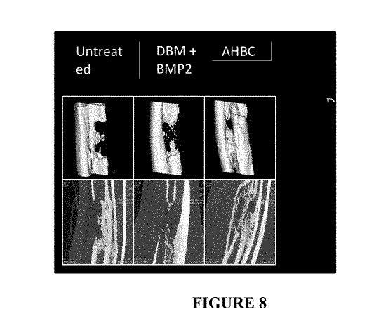

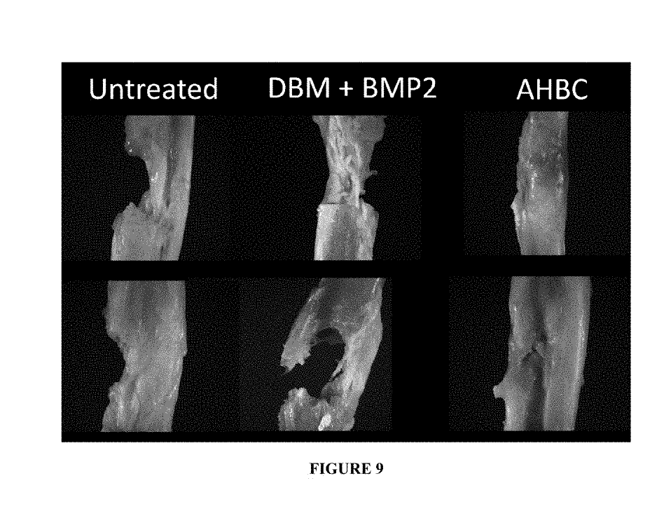

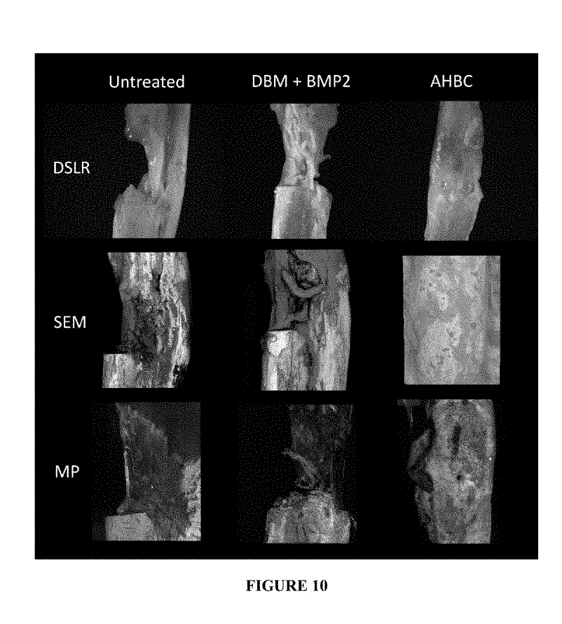

The AHBC treated group resulted in bone formation. The images in FIGS. 6-8 show qualitative bone regeneration with AHBC treatment. The images in FIGS. 9 and 10 also show qualitative bone regeneration with AHBC treatment. AHBC also shows structural integrity and when separated from the radius, shows disassociation to the radius. FIGS. 9 and 10 demonstrate AHBC treatment resulted in bone formation similar to native bone. Moreover, FIGS. 9 and 10 also demonstrate subjects receiving AHBC treatment show increased bone growth compared to that of the untreated animals with the bone defects. Accordingly, these results demonstrate that the osseous-derived compositions disclosed herein are useful in methods for promoting bone regeneration in a subject in need thereof.

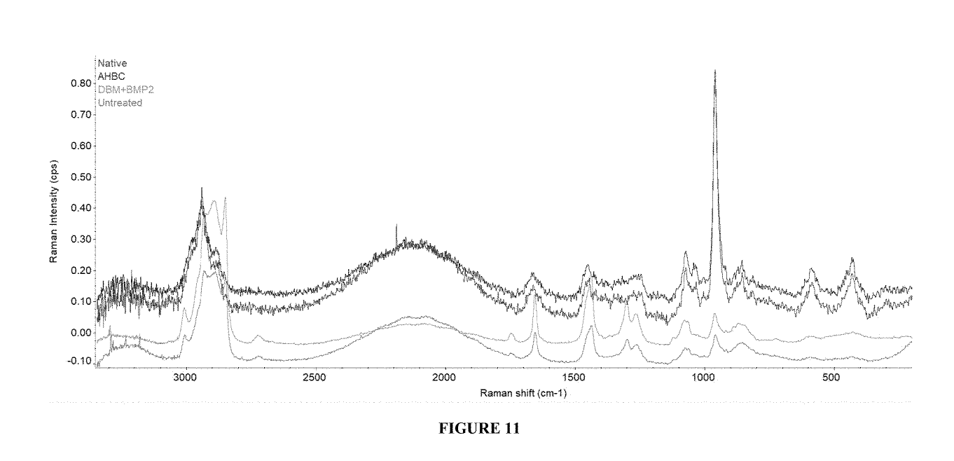

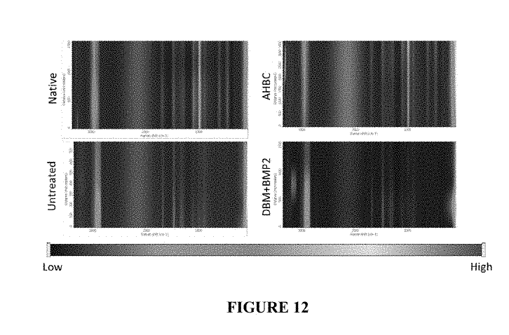

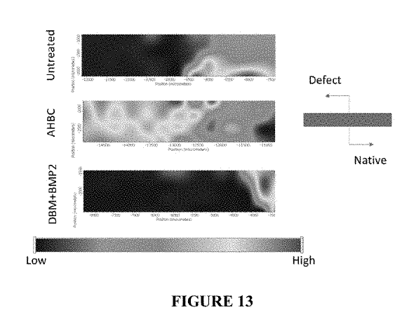

Average surface point spectra from native bone, untreated defects, and the AHBC treated group were compared at the phosphate peak location as shown in FIG. 11. Surface line scans from native bone, untreated defects, and the AHBC treated group were collected and show phosphate peak lines as shown in FIG. 12. Surface area scans from native bone, untreated defects, and the AHBC treated group were compared as shown in FIG. 13. The phosphate peak at 961 cm⁻¹ is an indication of the bone mineral hydroxyapatite formation and the intensity is related to the concentration. As shown in FIGS. 11-12, the AHBC treated group shows high phosphate intensity resembling native bone mineral and indicating bone mineral formation as in native bone.

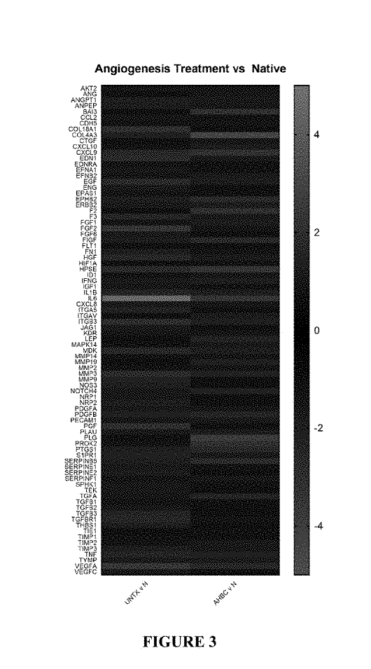

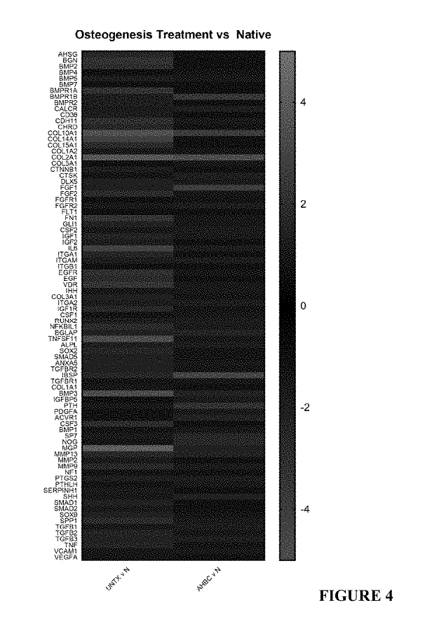

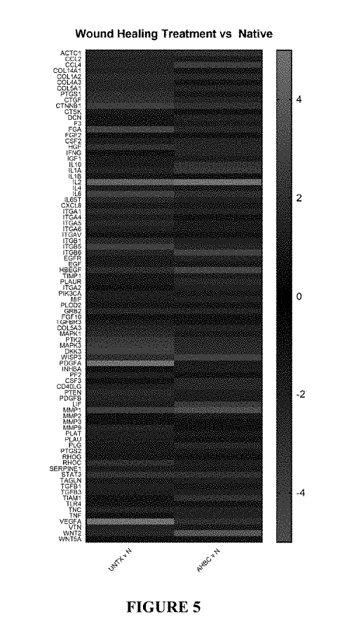

Gene expression profiles for defects with AHBC treatment were compared to native tissue and AHBC (Group 3) was also compared to untreated wounds. FIG. 3 shows a heat map displaying fold change in gene expression of angiogenesis factors for the AHBC treated group compared to native bone. FIG. 4 shows a heat map displaying fold change in gene expression of osteogenesis genes for the AHBC treated group compared to native bone. FIG. 5 shows a heat map displaying fold change in gene expression of wound healing genes for the AHBC treated group compared to native bone. The comparison of AHBC versus native tissue resulted in four downregulated genes (IL2, MYOSIN2, ITGB5, and STAT3) out of 252 genes tested, showing that 98.4% of genes tested are similar in AHBC treatment and native tissue. Therefore, AHBC treatment resulted in a healed wound that is very similar to native bone at the gene expression level. Accordingly, these results demonstrate that the osseous-derived compositions disclosed herein are useful in methods for promoting bone regeneration in a subject in need thereof.

### Example 6: Rabbit Spinal Study

The goal of the study was to determine the spinal fusion efficacy in defect healing of an osseous-derived composition (e.g., AHBC). The defect model consisted of 36 New Zealand White rabbits. A median incision at the level of the iliac crest was made and the iliac crests were exposed bilaterally. Approximately 2-2.5 cm³ of bone was removed from each iliac crest. This bone was processed to obtain the osseous tissue interface and to create a stimulated composition comprising an aggregate of living core potent cellular entities and supportive entities where the living core potent cellular entities express a sequence of LGR4, LGR5, and/or LGR6. Next, paramedian facial incisions were made to gain access to the transverse processes. Once through fascia, blunt dissection with a finger was used in order to develop the area between muscles. Blunt dissection was used to further move longissimus muscle fibers off the transverse process from both the cephalad and caudal vertebrae at the fusion level. Next, decortication of the transverse process was performed using a high speedburr. Once the cephalad and caudal transverse process were properly decorticated, the osseous-derived composition was carefully applied to the areas of decortication. This process was then repeated on the contralateral side. The fascia was closed on top and the remaining layers of tissue and skin were closed in layers.

Table 2 shows the treatment groups:

TABLE 2

| Group # | n | Recipient | Treatment |
|---|---|---|---|
| 1 | 6 | New Zealand White | Autograft |
| 2 | 6 | New Zealand White | DBM + BMP2 |
| 3 | 6 | New Zealand White | AHBC |

Raman Spectroscopy:

A confocal Raman microscope (Thermo Fisher Raman DXR with a 10× objective and a laser wavelength of 785 nm (28 mW laser power) was used to collect spectra along the cross section of the spinal fusion mass. A 25-urn slit aperture was used to collect a spectral range between wavenumbers 500-3500 cm⁺¹. The estimated resolution was 2.3-4.3 cm⁻¹. Spectral data was collected using an exposure of 1 s with a signal to noise ratio of 300 to ensure the collected spectra represent the bulk material.

Raman spectroscopy analysis was performed using OMNIC (Thermo Scientific) software for Dispersive Raman. Features available on OMNIC software were used to remove background fluorescence from all surface point scan spectra using 6^{th} order polynomial baseline fitting. Surface point spectra collected from each specimen were normalized and averaged to represent an individual animal. Overall group averages were calculated using average spectra from each individual animal within the group. OMNIC Chemigrams for cross sectional area scans were created using ranges 950-965 cm⁻¹ for hydroxyapatite.

Results:

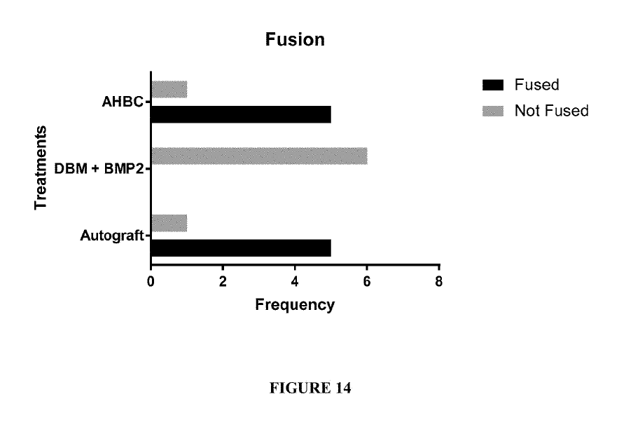

The AHBC treated group showed the highest frequency of fusion and was the same as autograft. The chart in FIG. 14 illustrates the spinal fusion frequency.

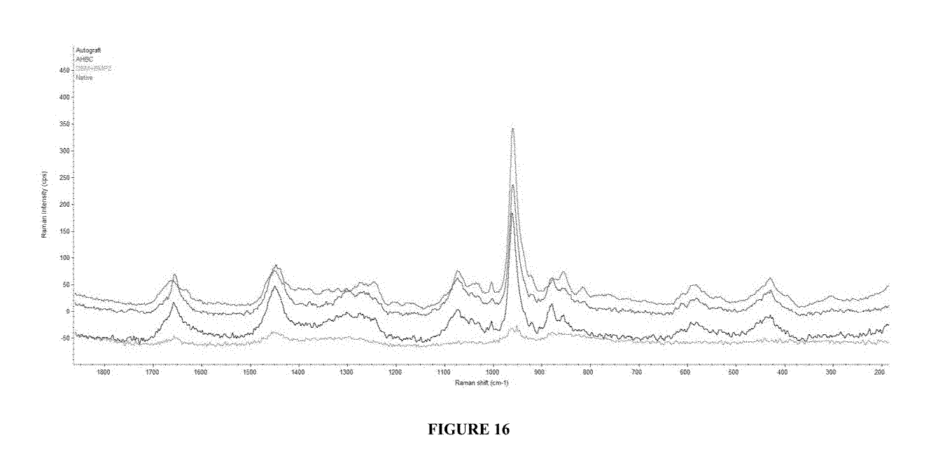

Average point spectra from native bone and treated groups were compared at the phosphate peak location as shown in FIG. 16. The phosphate peak at 961 cm⁻¹ is an indication of the bone mineral hydroxyapatite formation and the intensity is related to the concentration. The AHBC treated group shows high phosphate intensity resembling native bone mineral and indicating bone mineral formation as in native bone.

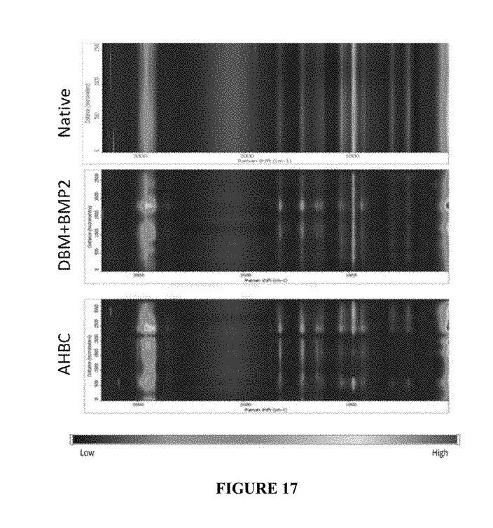

Cross section line scans were collected to demonstrate distribution of bone mineral along a certain distance as shown in FIG. 17. The hydroxyapatite peak intensity is represented as a line at 961 cm⁻¹. As shown in FIG. 17, the AHBC treated group shows high phosphate intensity resembling native bone mineral and indicating bone mineral formation as in native bone.

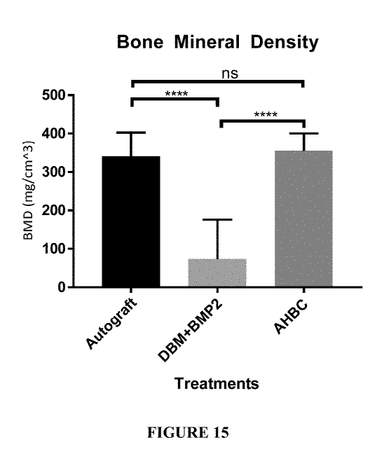

As shown in FIG. 15, the bone mineral density of the AHBC treated group was comparable to animals that received the autograft. Moreover, animals treated with AHBC show superior bone mineral density compared to animals that received treatment with DBM+BMP2. Accordingly, these results demonstrate that the osseous-derived compositions disclosed herein are useful in methods for promoting bone regeneration in a subject in need thereof.

### Example 7: Rabbit Cranial Study

The purpose of this study was to explore the capability of an osseous-derived composition (e.g., AHBC) to repair critical sized defects in the skull of a large animal rabbit model. 25 female New Zealand White rabbits aged to skeletal maturity of 7 months received two 8 mm parietal bone critical-sized defects. One defect served as an untreated control in each animal and the other defect was treated. Table 3 shows the treatment groups:

TABLE 3

| Group # | n | Recipient | Treatment | Control |
|---|---|---|---|---|
| 1 | 5 | New Zealand White | AHBC | Untreated |
| | | | | Critically |
| | | | | Sized Defect |
| 4 | 5 | New Zealand White | Autologous | Untreated |
| | | | Split Calvarial | Critically |
| | | | Bone Graft (Autograft) | Sized Defect |
| | | | (ABG) | |
| 5 | 5 | New Zealand White | DBM + BMP2 | Untreated |
| | | | (10 ug/ml) | Critically |
| | | | | Sized Defect |

A midline incision from the nasofrontal area to the anterior aspect of the external occipital protuberance was made to expose the periosteum. The periosteum was incised and reflected bilaterally using blunt dissection to expose the parietal calvarial bone surface. Paramedian 8 mm defects were made by carefully drilling with a trephine bore bit with copious irrigation with crystalloid. When needed, bone wax was used to obtain hemostasis within the created defect. Two total defects were made per rabbit with one on either side of the central sinus. Care was taken so as not to damage the dura mater or the underlying blood vessels and sinus.

After removal of the bone from the defect site, it was placed into sterile transport media and processed on-site into an osseous-derived composition (e.g., AHBC). Processing was performed on the osseous tissue interface to create a stimulated composition comprising an aggregate of living core potent cellular entities and supportive entities where the living core potent cellular entities express a sequence of LGR4, LGR5, and/or LGR6. Generally, AHBC was implanted into left defect but in cases of dural tears caused during defect creation or the use of bone wax to achieve hemostasis test article was deployed in the right defect. After application of test article into the treatment site the periosteum over the operative site was closed using non-absorbable suture. The soft tissue/muscle and skin was then closed using non-absorbable suture.

Split calvarial autografts were prepared by taking the calvarial disks removed during the creation of defect sites and burring down the inner table and cancellous components of the disk. The remaining outer table was then implanted into the defect site.

DBM+BMP-2 was prepared by combining (Human) DBM with 10 ug/mL of Bone Morphogenic Protein-2 (BMP-2). Defects were filled with DBM+BMP-2 using an equivocal volume as the amount of AHBC used for AHBC treated animals.

At the end of the study, tissues harvested included en-bloc skull. Downstream dissection of tissues included removal of overlying skin muscle and pericranium followed by en-bloc removal of cranial bone containing both defect sites.

CT scans were obtained 2 weeks after surgery and at the time of tissue harvest 8 weeks following surgery.

Imaging Methods:

Gross Imaging: DSLR photographs were acquired intra operative with a Canon 5DSR. Ex vivo images documented using the same setup with camera mounted on copy stand.

Vimago CT—The animals were scanned immediately post-operatively and every two weeks and at the end of the eight-week study using the Vimago CT with the following settings:

60 mA

80 kV

7 ms

Time—32 seconds

Resolution—200 um

Micro-CT (μCT): A Quantum GX2, PerkinElmer instrument was used to image all ex vivo rabbit crania specimens. Each specimen was imaged at 70 kV, 88 μA, FOV 36 mm, voxel size 90 μm, Al 0.5 CU 1.0 filter for 14 minutes to achieve best resolution. The images were analyzed with Analyze software version 12.0 (AnalyzeDirect, Overland Park, Kans., USA). The trabecular and cortical bone mineral densities (BMD) were determined using one phantom (25 mm QRM BMD phantom) with known densities of 50 mg/cm3, 200 mg/cm3, 800 mg/cm3, and 1200 mg/cm3 of hydroxyapatite. Thresholds were set at were set at 539 Hounsfield units, 294.34 mg/cm3.

Statistical analysis was performed using GraphPad Prism 7. A Dunnett's multiple comparison test was used to determine statistically significant differences among groups. Either the native or untreated groups were used as the control in the Dunnett's multiple comparison test.

Second Harmonic Generation (SHG) Imaging: Second harmonic generation imaging was performed using a Leica SP8 multiphoton confocal microscope equipped with a Chameleon tunable two photon laser tuned to 880 nm using a 10×0.40 NA objective. Signals were detected using Leica HyD detection system and converted to TIF format using Leica application Suite X software.

Confocal Fluorescent Imaging: Confocal fluorescent imaging was performed using a Leica TCS SP8 single photon confocal microscope. Samples were imaged with a 10×0.40 NA objective. Samples labeled with NucBlue (Catalog #: R37605, Thermofisher, Eugene, Oreg., USA), Osetoimage Mineralization Assay (Catalog #: PA-1503, Lonza, Walkersville, Md., USA), and Actin-555 R37112, Thermofisher, Eugene, Oreg., USA) were visualized using 405 (Diode), 488 (Argon), 514 (Diode), and 633 (HeNe) laser lines and signals were detected using Leica HyD and PMT detectors. Images were viewed and converted to TIF format using Leica application suite X software.

Compound microscopy: Both defects excised en bloc were imaged on Zeiss V16 compound microscope 503 camera. Z stacked and tiled images of entire en bloc top and bottom acquired. Individual defects top and bottom were also acquired. Regions of interest acquired at varying magnifications dependent on characteristics that deviated from surrounding native bone.

Compound microscopy was performed on 10% normal buffered formalin (NBF) fixed crania cross sections using a Leica M205 FA compound microscope. Samples were viewed with a 0.63× planapo lens at a 2× zoom and images were collected using a Leica DFC7000 T camera.

Scanning Electron Microscopy Imaging: Scanning electron microscopy was performed using EVO LS10 ESEM (SEM). Samples were imaged with high definition back scatter detector (HDBSD) in addition to an Extended Range Cascade Current Detector (C2DX). Images were captured and compiled using Zeiss SmartSEM and SmartStitch software (Zeiss SmartSEM: Version 6.02, Zeiss SmartStitch: Version V01.02.09). Final stitching of images was completed using FIJI (Version 1.52e).

Raman Spectroscopy:

A confocal Raman microscope (Thermo Fisher Raman DXR Microscope) with a 10× objective and a laser wavelength of 785 nm (28 mW laser power) was used to collect spectra. A 25-um slit aperture was used to collect a spectral range between wavenumbers 500-3500 cm-1. The estimated resolution was 2.3-4.3 cm-1. Spectral data was collected using an exposure of 1 s with a signal to noise ratio of 300 to ensure the collected spectra represent the bulk material. For surface point scans, a total of 2-5 spectra were collected from arbitrary positions across the top surface of the defect. For surface line scans, 6 spectra were collected with 200 um spacing between each point of collection. In addition to point and line scans, cross sectional area scans were collected for each animal defect. Area scans consisted of full thickness cross sections covering an area between 3-15 mm2 with 100-320 points of collection.

Raman spectroscopy analysis was performed using OMNIC (v.32, Thermo Fisher) software for Dispersive Raman. Features available on OMNIC software were used to remove background fluorescence from all surface point scan spectra using 6th order polynomial baseline fitting. Surface point spectra collected from each specimen were normalized and averaged to represent an individual animal. Overall group averages were calculated using average spectra from each individual animal within the group. OMNIC Chemigrams for cross sectional area scans were created using ranges 950-965 cm-1 for hydroxyapatite and 880-840 cm-1 for collagen.

Results:

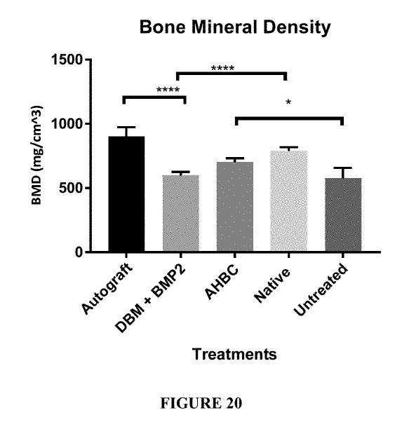

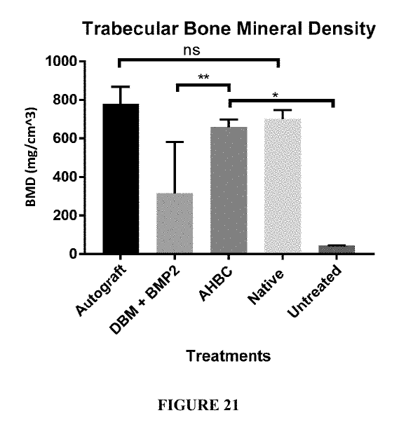

Bone mineral density measurements demonstrated that treatment with AHBC resulted in a similar bone mineral density to native bone. FIG. 20 shows bone mineral density of the AHBC treated group was comparable to that of native bone. FIG. 21 shows trabecular bone mineral density of the AHBC treated group was comparable to that of native bone.

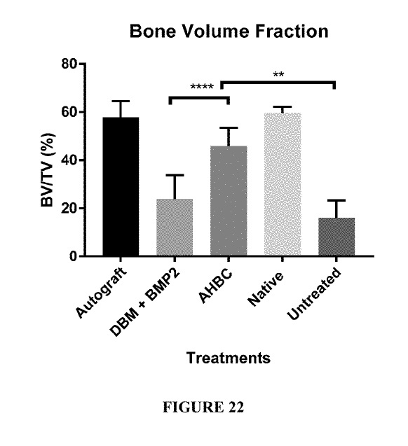

AHBC resulted in a bone volume to tissue volume percentage similar to native bone. FIG. 22 shows bone volume to tissue volume percentage of the AHBC treated group was comparable to that of native bone.

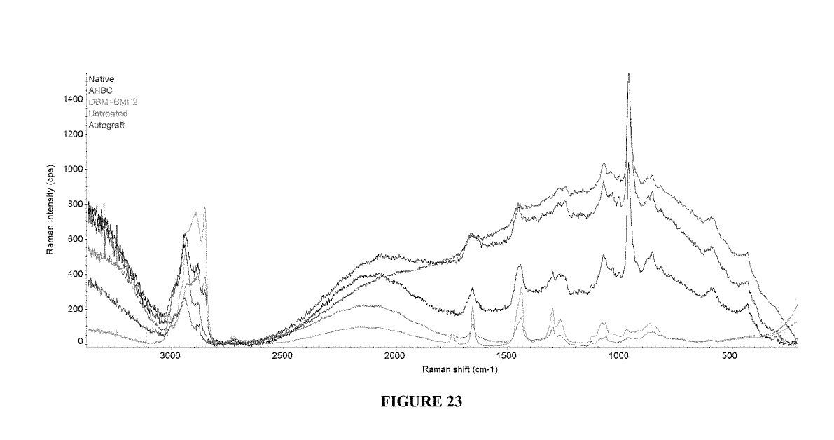

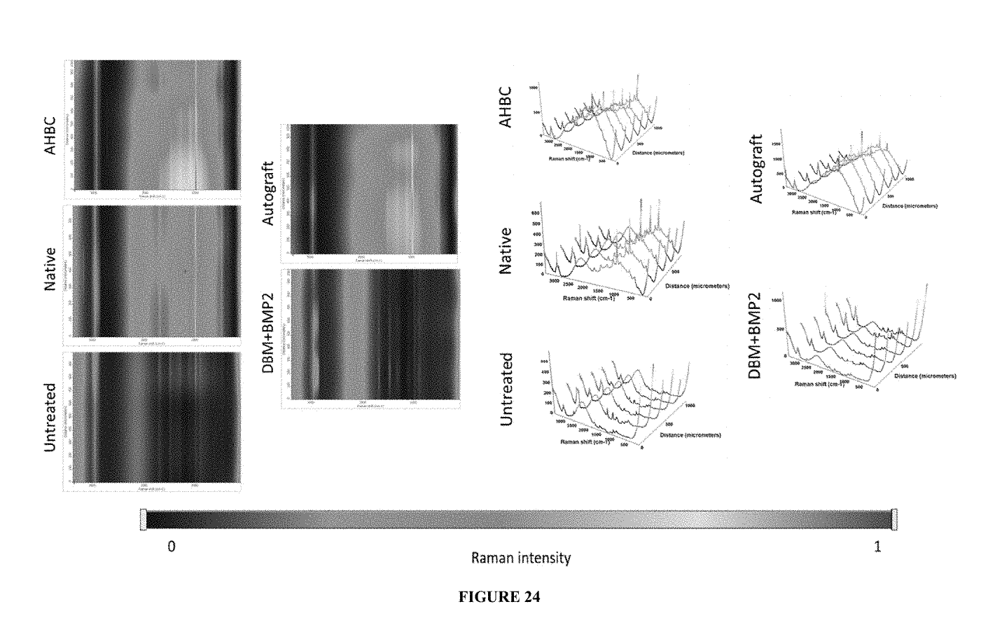

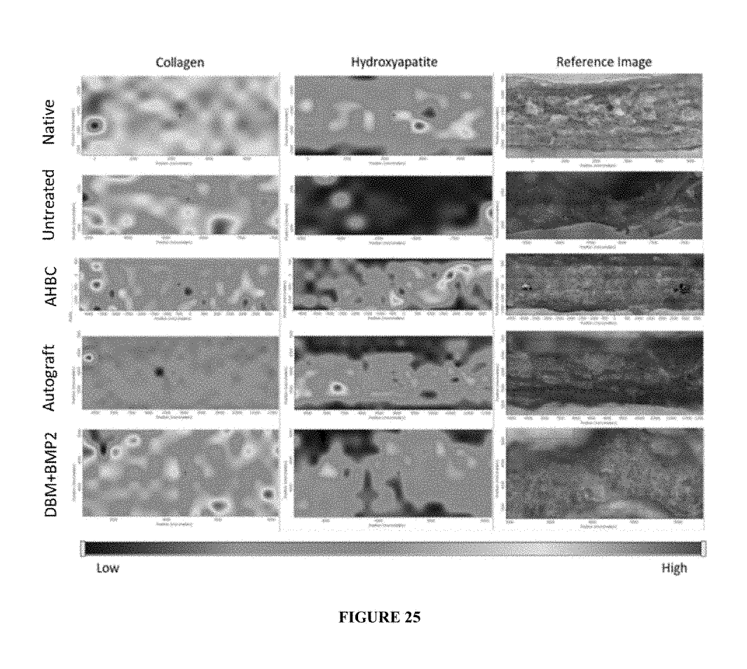

Raman spectroscopy indicated the presence of hydroxyapatite in the average point scans, surface line scans, and area scans indicating bone mineral formation for the AHBC treatment. Average surface point spectra from native bone, untreated defects, and the AHBC treated group were compared at the phosphate peak location as shown in FIG. 23. Surface line scans from native bone, untreated defects, and the AHBC treated group were collected and show phosphate peak lines as shown in FIG. 24. Cross-sectional area scans from native bone, untreated defects, and the AHBC treated group were collected and show hydroxyapatite distribution in FIG. 25. The phosphate peak at 961 cm⁻¹ is an indication of bone mineral hydroxyapatite formation and the intensity is related to the concentration. As shown in FIGS. 23-25, the AHBC treated group shows phosphate intensity indicating bone mineral formation. In FIG. 24, hydroxyapatite lines are visibly similar to native for the AHBC treated group.

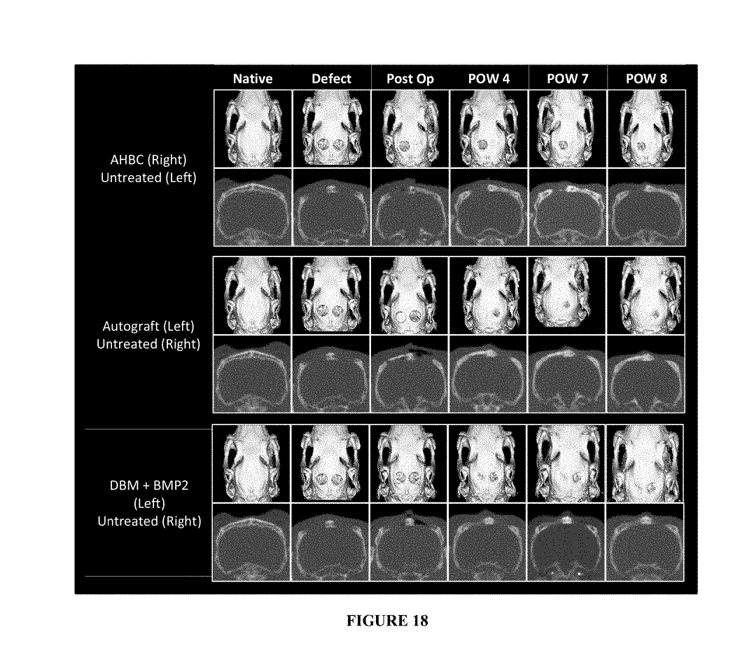

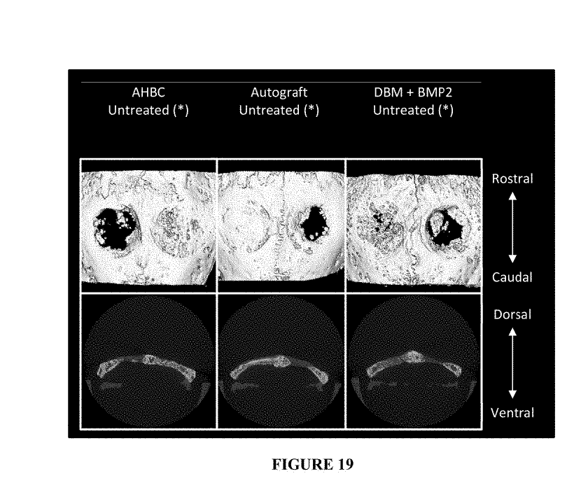

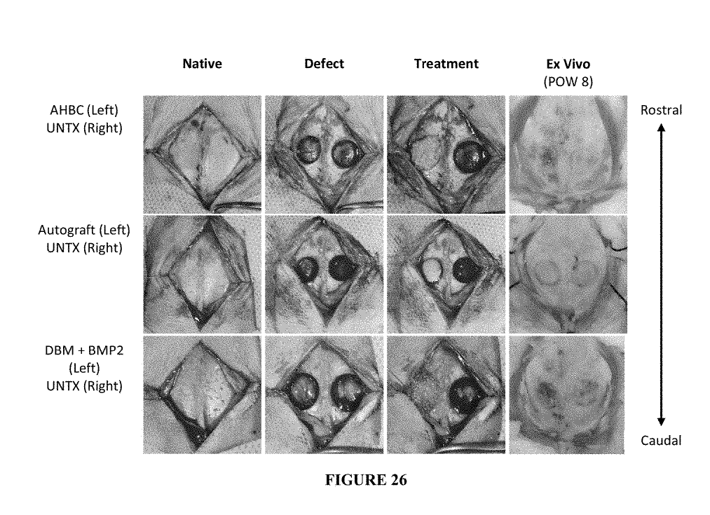

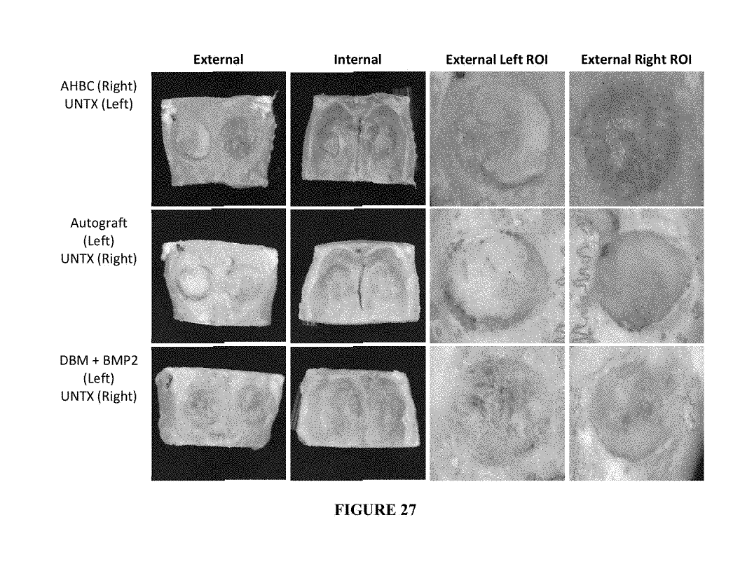

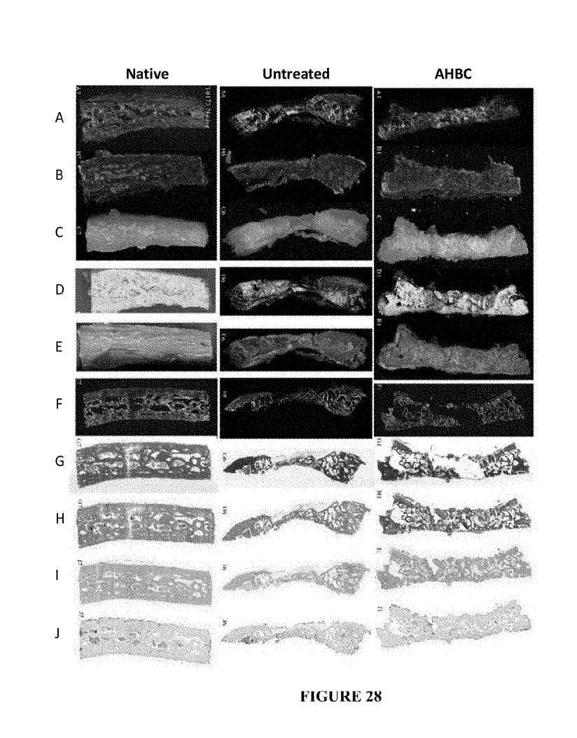

The AHBC treated group resulted in bone formation. CT scans in FIGS. 18 and 19 show bone regeneration with AHBC treatment. The images in FIGS. 26-28 also show bone regeneration with AHBC treatment. AHBC treatment resulted in similar craniotomy closure compared to ABG with minimal, poorly formed bone observed with DBM+BMP2 treatment on CT imaging and gross inspection. Ultrastructural analysis by scanning electron microscopy and second harmonic resonance imaging showed AHBC treated defects developed cortical bone complete with lacunae and organized collagen structure. Mechanical, compositional, and structural analysis demonstrated AHBC-formed bone was similar to ABG (p<0.05), whereas DBM+BMP2 and untreated controls had properties that indicated fibrosis with minimal bone formation. These results demonstrate the osseous-derived compositions disclosed herein are useful in methods for promoting bone regeneration.

### Example 8: Differential Gene Expression Between Osseous-Derived Composition and Native Osseous Tissue (Rabbit)

Qiagen RT2 PCR profiler arrays were used to assess the molecular response to processing. Processing was performed on an osseous tissue interface to create a stimulated composition comprising an aggregate of living core potent cellular entities and supportive entities where the living core potent cellular entities express a sequence of LGR4, LGR5, and/or LGR6. Osteogenesis, angiogenesis, and wound healing pathways were assayed. Differentially expressed genes were determined using a Student's t-test to test the association between gene expression in pre- and post-processed samples. Enrichment for low p-values (P<0.05) were assessed by permutation. Specific pre- and post-processing signatures were detected in osteogenic, wound healing, and angiogenic pathways (Empirical P<0.05). Table 4 shows the treatment groups.

TABLE 4

| Group | | # of |
|---|---|---|
| ID# | Group | samples |
| 1 | Pre-processing | 4 |
| 2 | Post- processing | 5 |

Sample Collection: Tissue was collected from four pre- and five post-processed rabbit cranium. Tissue was collected in AllProtect (Qiagen), held at 4 C for 24 hr, and then moved to −80 C for storage until RNA extraction was performed.

RNA Extraction: Lysis of tissue was performed with PowerLyzer (Qiagen) for two cycles of 45 seconds at 3500 rpm with a 30 second dwell time between cycles. RNA was purified from the resulting tissue lysate using RNeasy Plus Universal Mini Kit (Qiagen). RNA was quantified using Nanodrop Lite (ThermoFisher Scientific).

Reverse Transcription and qRT-PCR: 800 ng of RNA was reverse transcribed to cDNA using RT2 First Strand Kit (Qiagen). Resulting cDNA was used as the template for RT2 PCR Profiler plates which were run according to manufacturer instructions (Qiagen) on a QuantStudio 12K Flex or QuantStudio 3 (Applied Biosystems, ThermoFisher Scientific).

Statistical Analysis: Data from these runs was analyzed comparing pre- and post-samples. qPCR data was analyzed using Rv3.5.1. Differential expression of Qiagen pathway genes (Osteogenesis, Angiogenesis, and wound healing) was determined using a Student's t-test (two-tail distribution and equal variances between the two samples). Fold regulation of individual genes was calculated using the delta-delta Ct method. To assess low p-value enrichment (P<0.05) in each of the three arrays tested, we permuted the pre/post phenotype 10,000 times and then used the Student's t-test to test the association between gene expression and each permuted phenotype. Empirical p-values for enrichment were generated by recording the number of times the proportion of p-values less than 0.05 was greater in the permuted dataset than the observed data.

Results: Gene expression profiles were generated for pre- and post-samples using Qiagen RT2 PCR pathway arrays. Statistical significance between each group and native were determined using a Student's t-test. Hierarchical clustering of molecular signatures from each sample as shown in FIG. 29 demonstrate that pre- and post-processing samples cluster into distinct groups for all the pathways tested indicating molecular pathways are altered after processing into an osseous-derived composition as disclosed herein. None of the genes were significant after testing for multiple correction within each panel (P<5.95×10⁻⁴), likely due to the small sample size of the study. Testing was additionally conducted for enrichment of low p-values (P<0.05) in our dataset relative to 10,000 permutations. Permutations simulate the number of low p-values you expect to find by chance. All panels were at least modestly enriched (P<0.05) for low p-values, i.e., the number of p-values less than 0.05 is greater than one would expect by chance.

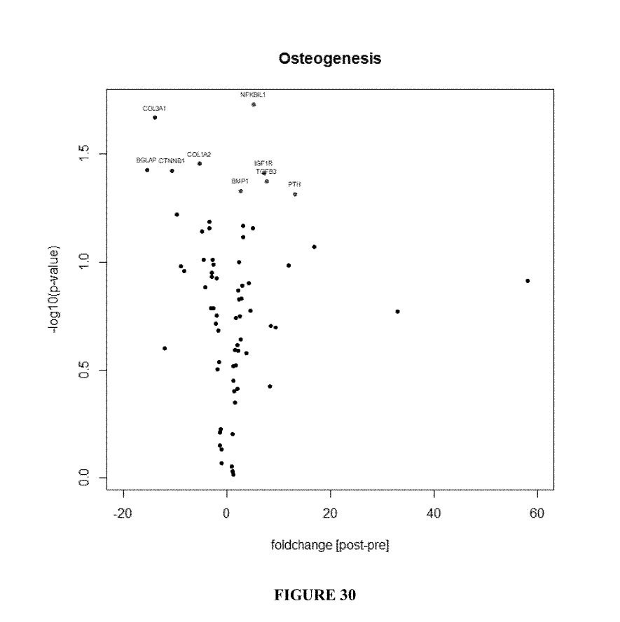

Enriched genes for each panel are shown in FIGS. 30-32. In FIGS. 30-32, fold change is shown on the x-axis. P-values (y-axis) correspond to differences in gene expression between pre- and post-cranium samples. Colored dots indicate a difference with a P<0.05. Increased and decreased expression of corresponding genes in post-relative to pre-processed cranium is shown by red and blue dots, respectively. Black dots indicate sites with a P<0.05.

Osteogenesis pathways were modestly enriched for low p-values (Empirical P=0.016). Thirteen percent of genes (N=9) were differentially expressed. Among the largest increase in expression was Parathyroid hormone (PTH; 13× increase), which has been shown to enhance osteogenesis in human mesenchymal stem cells. See Kuo S-W, Rimando M G, Liu, S, Lee O K. Intermittent Administration of Parathyroid Hormone Enhances Osteogenesis of Human Mesenchymal Stem Cells by Regulating Protein Kinase Cδ. Int J Mol Sci. 2017; 18(10). This is consistent with the observed increase in BMP/TGF-β signaling which is known to be enhanced by PTH and also inhibit Wnt/β-catenin signaling (β-catenin [CTNNB1]/gamma-carboxyglutamic acid [BGLAP] reduction). See Yu B, Zhao X, Yang C, Crane J, Xian L, Lu W, Wan M, Cao X. PTH Induces Differentiation of Mesenchymal Stem Cells by Enhancing BMP Signaling. J Bone Miner Res. 2013; 27(9):2001-2014; Wany Y, Li Y-P, Pulson C, Shao J-Z, Zhang X, Wu M, Chen W. Wnt and the Wnt signaling pathway in bond development and disease. Fron Biosci. 2014; 19:379-407. Wound healing pathways were also enriched for low p-values (P=0.0072). Fourteen percent (9/63) of genes are modestly different between pre- and post-treatment. The majority of these genes (8/9) have reduced expression after processing suggesting processing reduces signaling in wound healing pathways. For example, connective tissue growth factor, among other molecules that are associated with growth are reduced after processing. Increased expression of TLR4, a pathogen associated pattern recognition receptor, after processing indicates activation of immune surveillance mechanisms due to processing of the samples. Angiogenesis pathways demonstrate the largest amount of enrichment (P<1×10⁻⁵) for modest p-values with 24% of pathway genes (19/80 genes) associated with disruption. The majority of these genes (16/19) increase expression upon processing, suggesting that processing increases angiogenic signaling. The largest fold increase is 189× for thymidine phosphorylase, a gene that promotes angiogenesis. Additional pro-angiogenic molecules are also observed (TGF-α, TGF-βR1, EFNA1).

Accordingly, the osseous-derived compositions as disclosed herein are useful for promoting bone regeneration in a subject in need thereof.

### Example 9: Differential Gene Expression Between Hepatic-Derived Composition and Native Hepatic Tissue (Mouse)

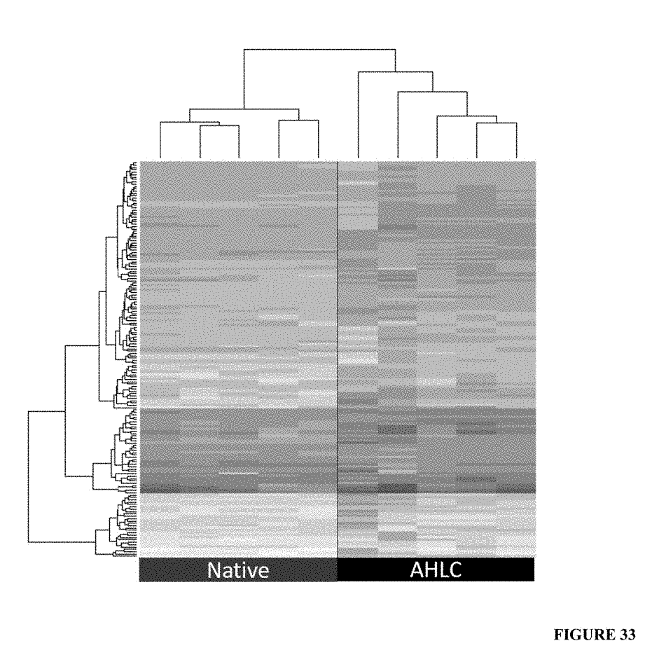

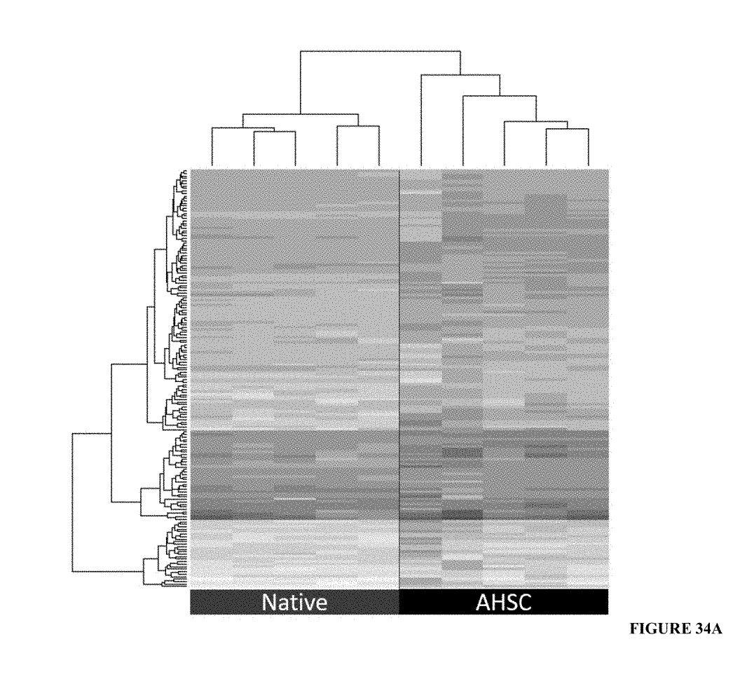

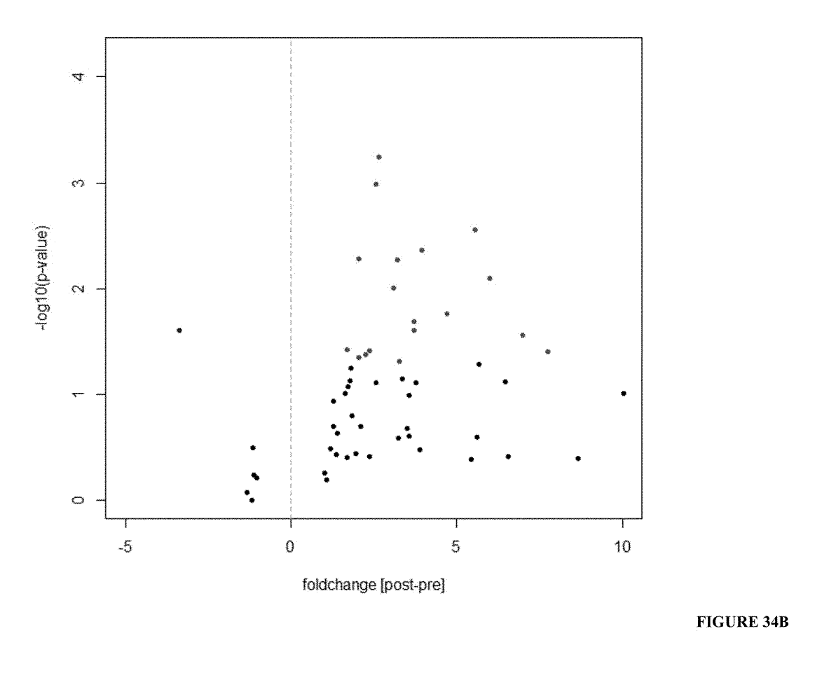

FIG. 33 shows a heatmap representative of altered molecular pathways in a hepatic-derived composition as disclosed herein versus native hepatic tissue. Dark red and yellow are associated with the highest and lowest levels of gene expression, respectively. This targeted transcript assessment indicates the presence of distinct gene expression profiles for native and processed hepatic (AHLC) samples.

### Example 10: Differences in Compressive Strength Between Various Tissue-Derived Compositions and Native Tissue

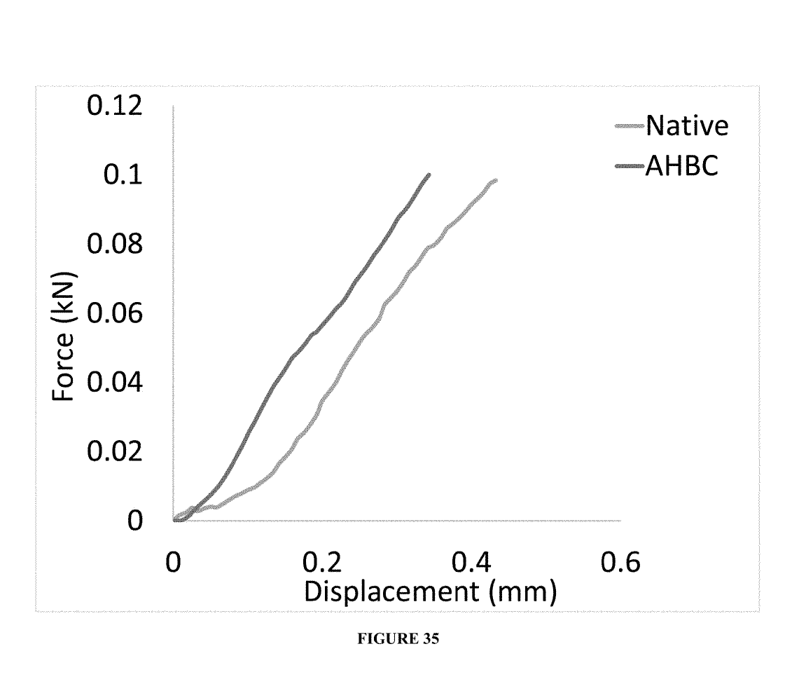

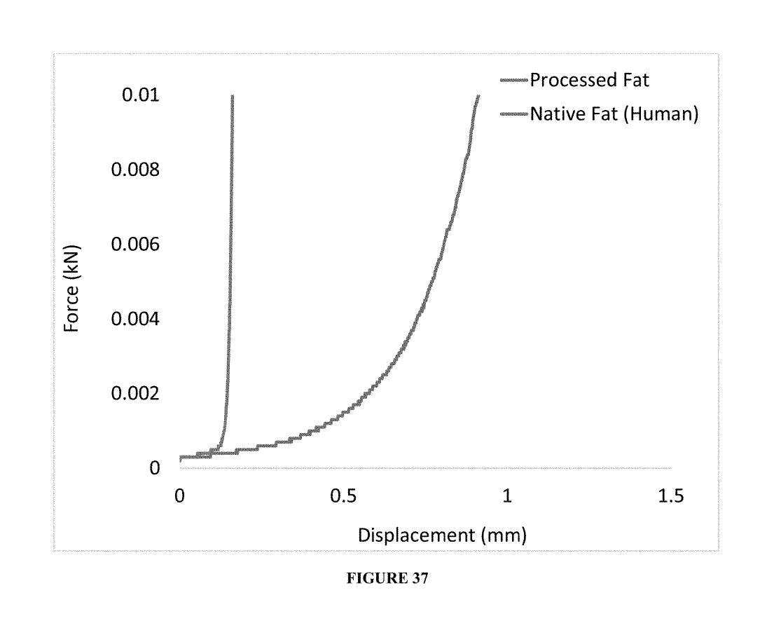

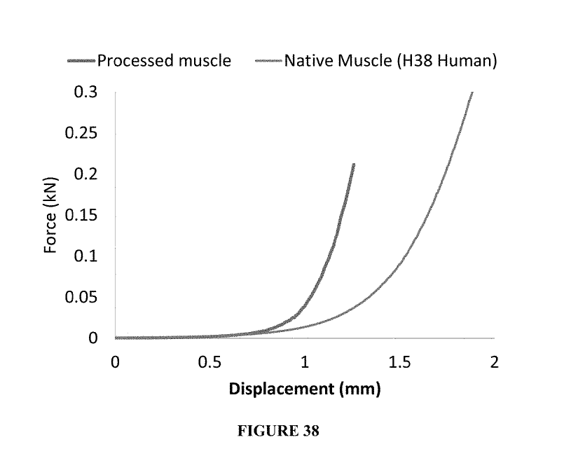

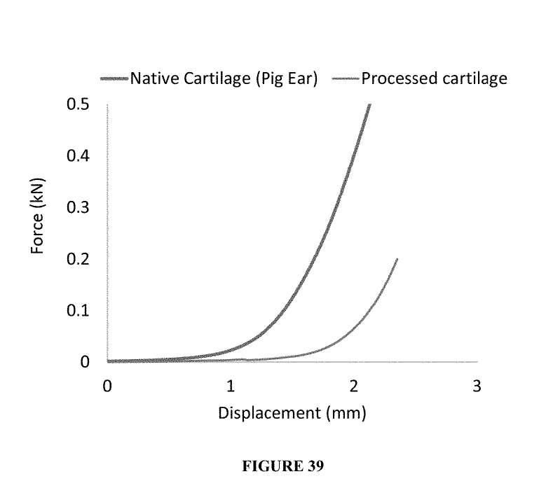

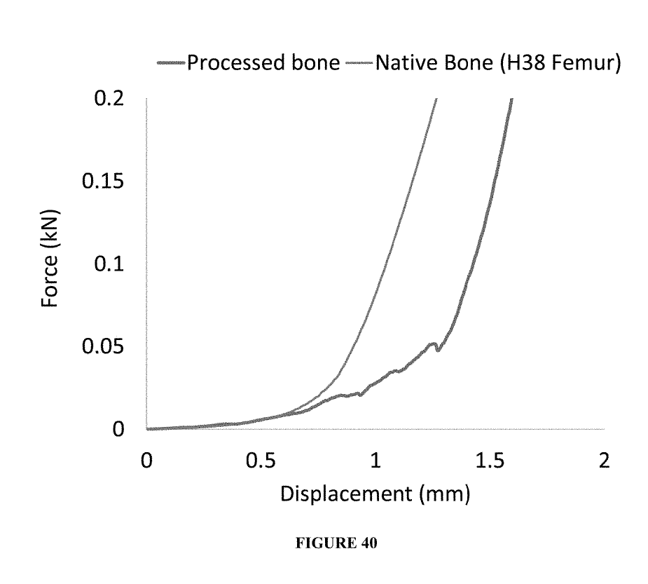

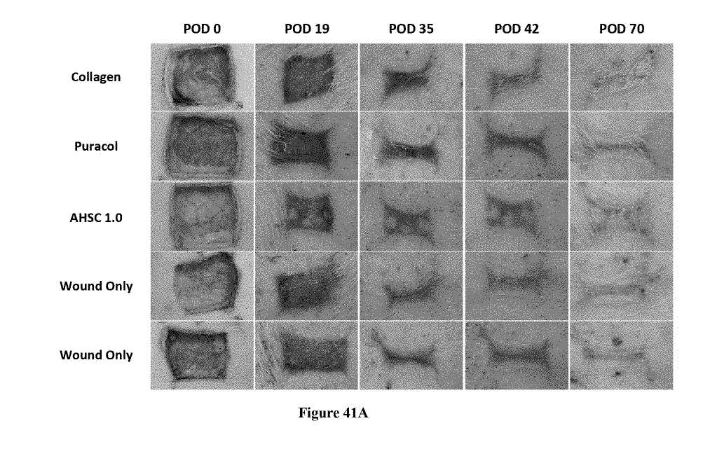

Each of rabbit long bone, fat (human), muscle (human), cartilage (pig), and bone (rabbit femur), respectively, were processed to obtain tissue interfaces and create stimulated compositions comprising an aggregate of living core potent cellular entities and supportive entities where the living core potent cellular entities express a sequence of LGR4, LGR5, and/or LGR6. Each of stimulated compositions were compared mechanically to the respective native tissue. A flat plate used for compression testing. Instron 3343 with a 1 kN load setting was used. FIGS. 35 and 37-40 show force versus displacement. The slope of the graphs defines the compressive strength. The force versus displacement response for native and processed tissue is non-comparable with different slopes (defined as modulus). This data shows that both native and processed tissue have different physical characteristics.

### Example 11: Differences in Hydroxyapatite Between Osseous-Derived Composition and Native Osseous Tissue (Rabbit)

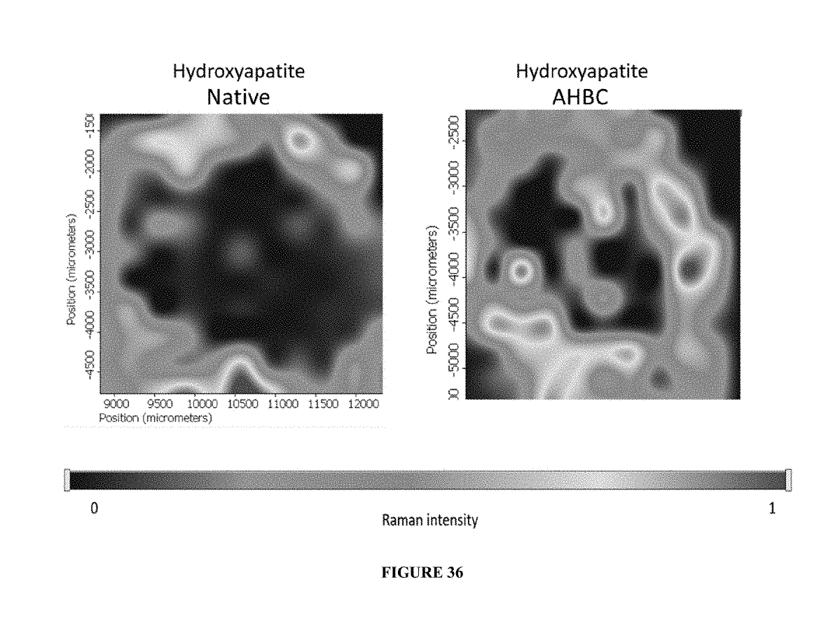

FIG. 36 shows Raman cross-sectional area scans of native rabbit long bone and rabbit long bone processed osseous-tissue interfaces to create a stimulated composition comprising an aggregate of living core potent cellular entities and supportive entities where the living core potent cellular entities express a sequence of LGR4, LGR5, and/or LGR6. The Raman scans were conducted as explained in Example 5. The Raman cross-sectional scans show hydroxyapatite distribution in FIG. 36. Intensity is related to the concentration of the bone mineral hydroxyapatite. As shown in FIG. 36, hydroxyapatite distribution is different between the processed and native tissues.

### Example 12: Differential Expression Between Cutaneous-Derived Composition and Native Cutaneous Tissue (Human)

FIG. 34A shows skin targeted transcriptome analysis assessing wound healing, stem cell, and cell surface marker pathways identify distinct signatures present in native skin relative to a processed composition (AHSC).

FIG. 34B shows a targeted stem cell assay indicates increased expression of stem cell markers in the processed composition (AHSC) relative to native skin suggesting activation of resident stem cells through processing and storage.

### Example 13: Preparation of Muscle-Derived Composition

Harvest rabbit thigh muscle using sharp dissection. Tissue is washed with an isotonic solution (e.g. 0.9% NaCl) for 5 minutes at 4° C. with gentle shaking. Muscle tissue interface separation is initiated by placing 10 grams of tissue into a 50 cc conical tube (Conical A) on ice and submerged in 20 mL of chilled HBSS. Collagenase Type IV (0.143 g), papain (0.019 g), dithiothreitol (0.0028 g) is added and Conical A is transferred to a warming bath warmed to 37.7° C. for 5 minutes. Vortex sample (300 VPM) and transfer contents to a culture dish and incubate at 37.7° C. in a 5% CO2 environment for 20-25 minutes, or until tissue dissociation is sufficient. Transfer composition to a 50 mL conical tube (Conical B), combine termination agent. Centrifuge composition at 1000 RPM for 10 minutes. Separate muscle tissue interfacing material from non-interfacing material consistent with standard methods including mesh filtration or precipitation. Centrifuge remaining composition including non-interfacing material at 1000 RPM for 5 minutes at room temperature. Transfer supernatant to a 50 mL (Conical C). Centrifuge Conical C at 30,000 RPM for 20 minutes. Discard supernatant. Wash Conical C with 10 mL of a biocompatible isotonic solution (1×HBSS, DMEM, RPMI, 0.9% NaCl, Lactated ringers). Combine 2:1 (v/v) with a biocompatible solution and add resulting combination with activated interfacing material to ensure sufficient hydration. Processing yields interfacing muscle tissues with reactive and stimulated components ranging in size from approximately 40 to 250 μm in diameter.

### Example 14: Preparation of Cartilage-Derived Composition

Rabbit articular cartilage is isolated and rinsed three times in phosphate buffered saline (PBS) at 4° C. Tissue is mechanically fractionated into segments with a volume ranging from 1 to 5 mm³. These tissues are then rinsed twice in PBS warmed to 37° C. and transferred to a 50 cc conical tube. PBS is pre-warmed to 37° C. in a 10:1 (v/v) volume to tissue volume with 2 mg/mL testicular hyaluronidase type 1-S and 0.25% trypsin/1 mM EDTA. Muscle tissues are incubated for 5-30 minutes. Tissues are rinsed with PBS twice. DMEM pre-warmed to 37° C. in a 10:1 (v/v) volume to tissue volume supplemented with 4.5 mg/ml glucose, 10 m MHEPES buffer, 100 U/ml penicillin, 100 μg/ml streptomycin, 1 mM sodium pyruvate, and 0.05 to 2% (w/v) collagenase type II is added and tissues are incubated for 1-20 hours at 37° C. while being centrifuged at 60 RPM. Resulting composition is centrifuged 1200 RPM for 10 minutes. Supernatant is transferred and saved for later use. The remaining reactive and stimulating interfacing and non-interfacing tissues are combined 1:1 (v/v) with PBS and separated using either mesh filtration, precipitation and/or mechanical isolation. Resulting reactive and stimulating interfacing elements of the processed tissue have a length of approximately 30 to 275 μm in longest axis.

### Example 15: Preparation of Adipose-Derived Composition

Subcutaneous, visceral, and/or brown rabbit adipose tissue is collected and rinsed with PBS with 100 U/ml penicillin, 100 μg/ml streptomycin chilled to 4° C. three times. Adipose tissues and interfaces are mechanically dissociated by methods known in the art, including centrifugation and/or vortexing (600 VPM) for 5 minutes for a total of 5 cycles. Adipose tissues are then combined with a biocompatible solution (DMEM, RPMI, PBS, 0.9% NaCl, lactated ringers) in a volumetric equivalent manner and centrifuged for 2000 RPM for 5 minutes. The oil/adipose layer is removed. This cycle is repeated for a total of 3 occurrences. Remaining reactive and stimulating interfacing tissue and non-interfacing components are resuspended with DMEM in a 0.5:1 (v/v) fashion and centrifuged at 500 RPM for 2 minutes. Reactive and stimulating interfacing tissue is separated via aspiration. Isolated active interfacing components range in volume from 1900 to 31,400 μm³.

### Example 16: Swine Skin Study

The purpose of this study was to evaluate development of neodermal growth, epidermal expansion, hair growth, and formation of vasculature within a full thickness wound bed treated with cutaneous-derived compositions (e.g., AHSC) and/or to evaluate wound closure with various preparations of a cutaneous-derived composition (e.g., AHSC) with and without various adjuncts.

Method:

12 nulliparous female conventional Yorkshire swine (30-40 kg at study initiation) were prepped in sterile fashion. Wound beds were created by excising full thickness skin using a combination of sharp dissection with a scalpel and electrocautery. Full thickness wound depth was verified by visualization of the muscular fascia underlying the predetermined wound area. A cutaneous-derived composition (e.g., AHSC) was created utilizing a portion of the excised full-thickness dermis from the created wound beds to create a stimulated composition comprising an aggregate of living core potent cellular entities and supportive entities where the living core potent cellular entities express a sequence of LGR4, LGR5, and/or LGR6.

Treatments were applied to wound beds and dressed. Wounds were allowed to heal for 18-200 days following surgery.

In Vivo Imaging Methods:

Gross Imaging: Gross photographs were acquired no less than weekly (during bandage change procedures) with a digital camera.

Vectra: Contour and contraction were measured by the utilization of a stereoscopic camera (Canfield Vectra H1) that renders the swine's back 3 dimensionally. Three images of the dorsal surface were taken in a cranial to caudal fashion. Data were recorded, and contraction measurements were made using Canfield VAM software.

Macroscopic Imaging: Macroscopic images of regions of interest were acquired using an olloclip lens (7×, 14×, 21× zoom) attached to an iPhone 6. For select swine, a dermascope (Canfield VEOS) was introduced for imaging regions of interest.

LDI: Moor full-field laser perfusion imager (moorFLPI-2) laser doppler imager (LDI, WO9740/09) images were acquired for swine SKN001-SKN012. One image was acquired per wound and for control purposes an image of native swine skin was acquired just above the most cranial wounds.

Microscopy: Compound microscopy was acquired using a Leica M205 FA microscope attached to a Leica DFC7000 T camera. Images were obtained with a 0.63× objective at 0.78, 1, and 2× zoom.

Histology & Tissue Imaging: Swine samples were collected in 10% normal buffered formalin and fixed overnight before being transferred to 70% ethanol. Samples were then processed in 70%, 95%, and 100% ethanol, cleared in xylene, and infiltrated with paraffin. Samples were then embedded in paraffin and sectioned into 4 μm slices and mounted on positively charged glass slides before being stained with hematoxylin and eosin, masson's trichrome, or periodic acid schiff. Stained slides were imaged using compound, SEM, confocal, and multiphoton microscopy to evaluate gross anatomical and microscopic ultrastructural features).

Confocal Fluorescent Imaging: Confocal fluorescent imaging was performed using a Leica TCS SP8 single photon confocal microscope. Samples were imaged with 10×0.40 NA objective. Samples labeled with NucBlue (Molecular Probes), Col-F (Immunochemistry Technologies), Actin-555 (Thermofisher), and Wheat-germ agglutinin-647 (Thermofisher) were visualized using 405 (Diode), 488 (Argon), 514 (Diode), and 633 (HeNe) laser lines and signals were detected using Leica HyD and PMT combination detection system.

Second Harmonic Multiphoton Imaging: Second Harmonic imaging was performed using a Leica SP8 multiphoton confocal microscope equipped with a Chameleon two photon laser and collected using a 10×0.40 NA objective.

Scanning Electron Microscopy Imaging: Scanning electron microscopy was performed using EVO LS10 ESEM. Samples were imaged with high definition back scatter detector (HDBSD) using 50× magnification at 15 kilovolts (kV) and 60 Pa.

Raman microscopy: A confocal Raman microscope (Thermo Fisher Raman DXR) with a 10× objective (N.A. 0.25) and a laser wavelength of 785 nm (28 mW of power at sampling point) was used to collect spectra. The estimated spot size on the sample was 2.1 μm and resolution was 2.3-4.3 cm-1. The confocal aperture used was a 25 μm slit, and spectra between wavenumbers 500-3500 cm-1 were collected. Raman spectroscopy analysis was performed using OMNIC software for Dispersive Raman. Proprietary features available in OMNIC (Thermo Scientific) software were used to remove background fluorescence from all the spectra using polynomial baseline fitting (6th order) and to normalize the spectra. Spectral data was collected using an exposure of 1 s with a signal to noise ratio of 300 to ensure specimen was homogeneous and the collected spectra represented the bulk material. Three data collection techniques were performed on native tissue and wounds using Raman spectroscopy including (1) cross section area, (2) cross section line, and (3) surface line scans. Cross section area and line scans include full thickness of wound or native skin. Cross section line scans include 7 points along the entire cross section of tissue. Surface line scans include 5 points spaced 20 μm apart along the surface of tissue.

Mechanical Characterization: The mechanical properties of the treated skin and native were studied in swine receiving up to 120 or 200 days of treatment. Three methods were used: Ballistometry (in vivo skin firmness), Tensile testing (ex-vivo elastic modulus) and Ultrasound Shear wave Elastography (in vivo elastic modulus).

Tensile testing: Skin slices across the treated wounds were tested for elastic strength using an electronic UTM (Universal testing machine) with 1 kN load capacity (Instron, MA, USA) at a constant crosshead velocity of 0.5 mm/min until 5 mm displacement was reached. The load and displacement values were recorded at 0.1 s intervals during testing. Treated skin samples and native skin samples were tested to determine the ex-vivo skin elastic modulus.

Ballistometer: The ballistometer (Diastron Ltd., Andover, UK) was applied to three adjacent but non-overlapping areas at each anatomical test site. Swine treated up to 200 days were tested with this technique in vivo. To ensure consistency of the data, a single investigator performed all ballistometer measurements. The ballistometer recorded three main parameters: indentation; alpha and coefficient of restitution (CoR) using the proprietary Diastron MApp software.

US SWE (Ultrasound Shear wave elastography): GE Ultrasound (GE Medical systems, Chicago, Ill.) with SWE capability was used to evaluate the in vivo elasticity of the treated wounds as compared to native skin. An ARF (acoustic radio frequency) pulse was used to generate shear waves in the tissue in a small (approximately 8-cm3) ROI. B-mode imaging was used to monitor the displacement of tissue due to the shear waves. The shear wave speed was used to evaluate the Young's modulus (kPa). The mean, maximum, minimum, and standard deviation of the shear wave speed (in centimeters per second) or the Young's modulus (in kilopascals) within the ROI were displayed. Young's modulus values throughout the treated wound were plotted as a surface map (Elastogram).

Molecular Analysis Methods:

Sample Collection: Tissue was collected from wounds and native skin following gross imaging. Tissue was collected in AllProtect (Qiagen), held at 4 C for 24 hr, and then moved to −80 C for storage until RNA extraction was performed.

RNA Extraction: Lysis of tissue was performed with TissueLyser LT (Qiagen) at 50 hz for 60 minutes. RNA was purified from the resulting tissue lysate using RNeasy Plus Universal Mini Kit (Qiagen). RNA was quantified using Nanodrop Lite (ThermoFisher Scientific).

Reverse Transcription and qRT-PCR-800 ng of RNA was reverse transcribed to cDNA using RT2 First Strand Kit (Qiagen). Resulting cDNA was used as the template for RT2 PCR Profiler plates which were run according to manufacturer instructions (Qiagen) on a QuanStudio 12K Flex or QuantSTudio 3 (Applied Biosystems, ThermoFisher Scientific). Data from these runs was analyzed comparing wounds to native tissue, and AHSC wounds to control wounds. qPCR data was analyzed by the online Qiagen Data Analysis Center using the delta-delta Ct method to determine fold-regulation of individual genes and student's t-test (two-tail distribution and equal variances between the two samples) to determine significance. The Qiagen plates used include: Extracellular Matrix and Adhesion Molecules (PASS-013Z), Stem Cell (PASS-405Z), WNT Signaling Targets (PASS-243Z), Inflammatory Cytokines and Receptors (PASS-011Z), Wound Healing (PASS-121Z).

Results:

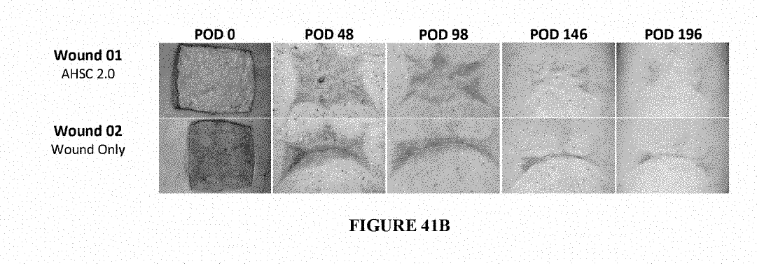

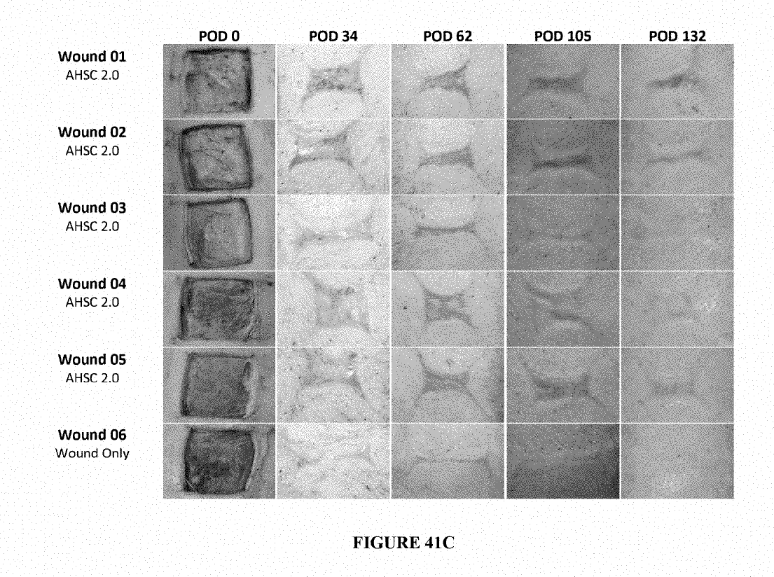

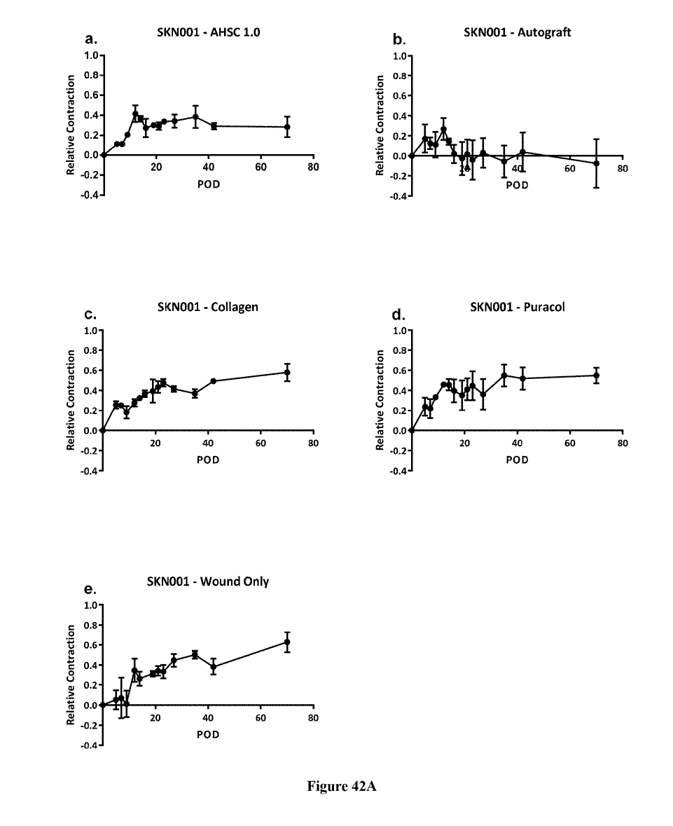

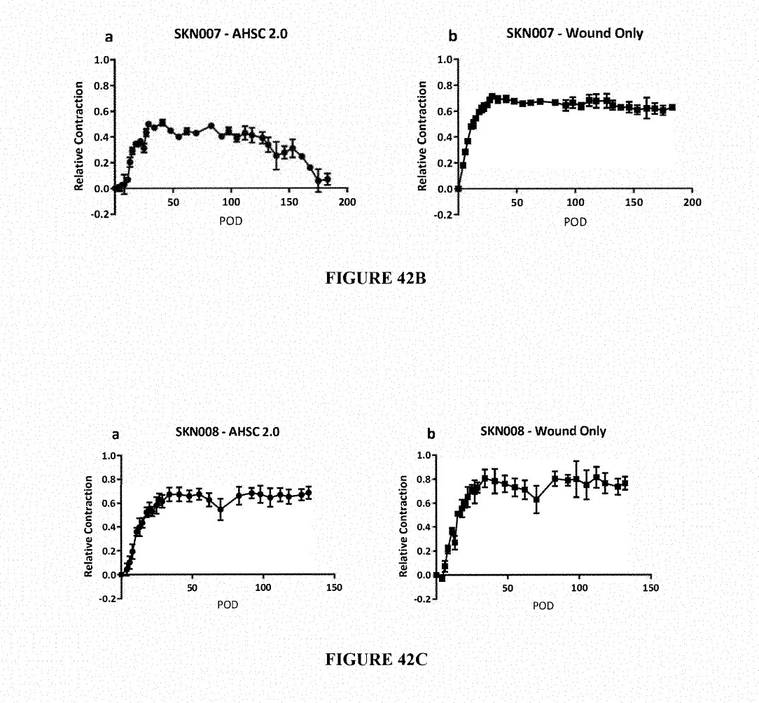

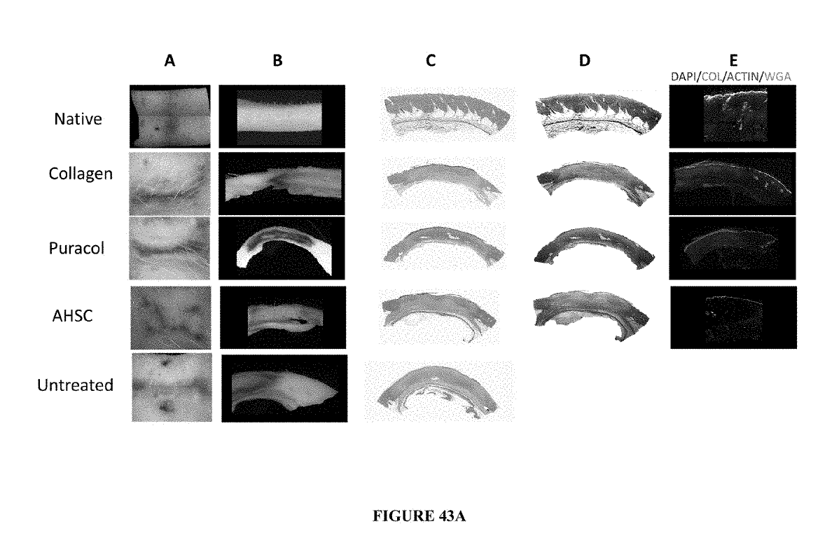

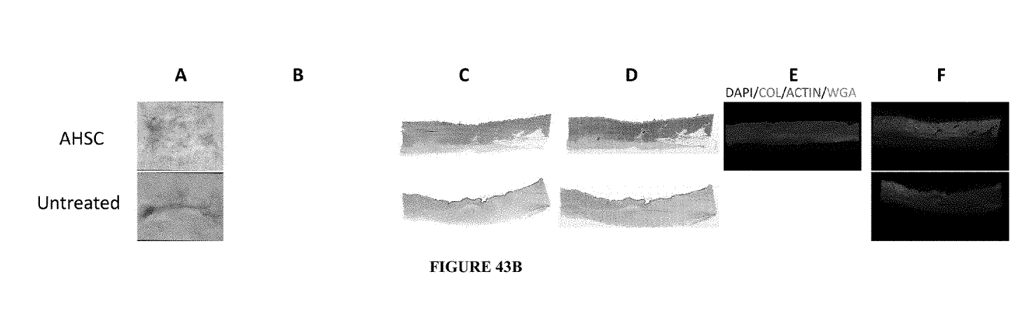

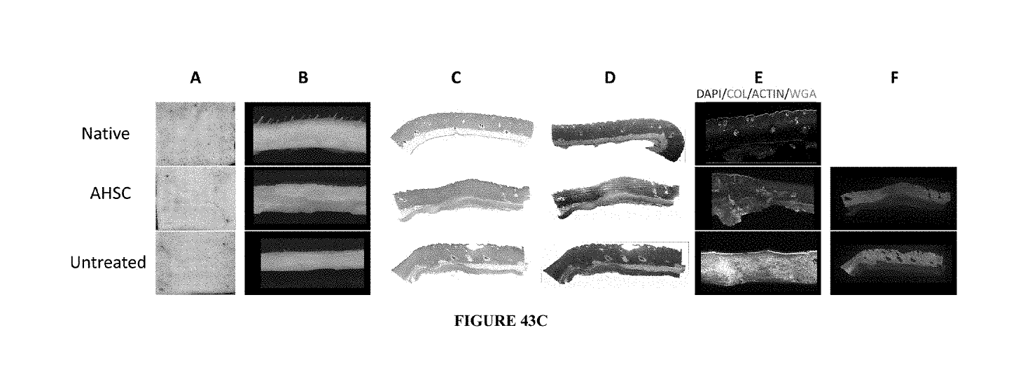

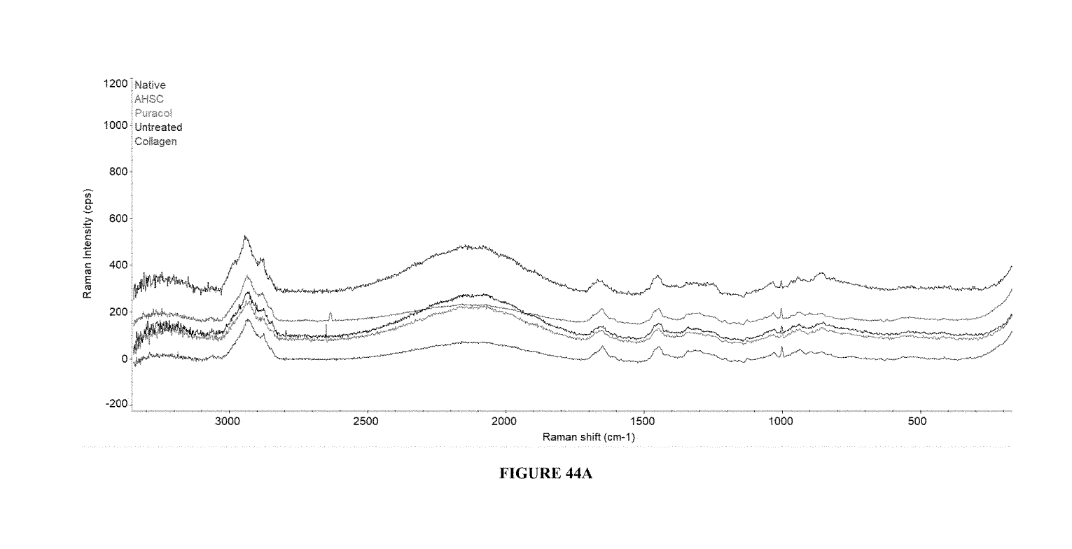

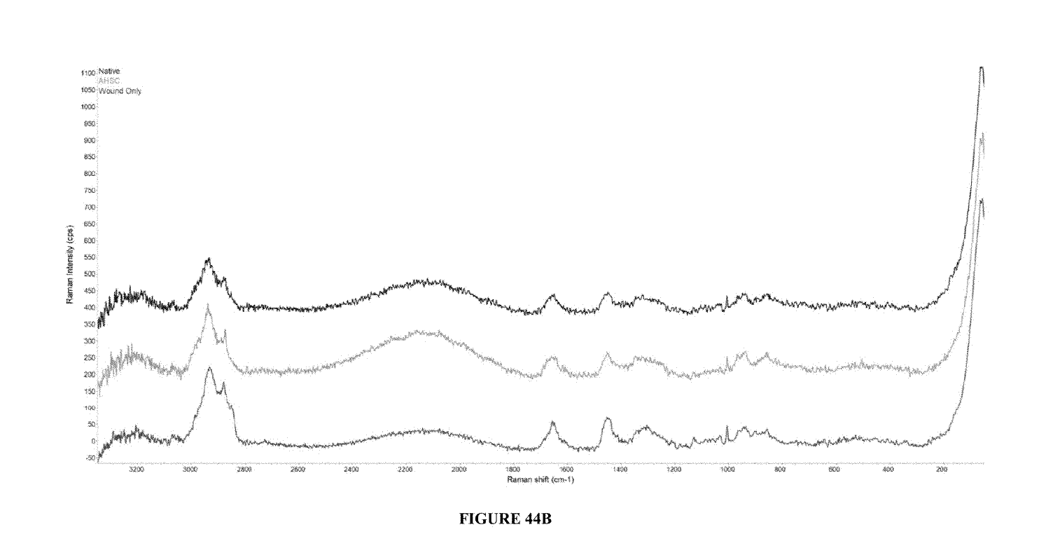

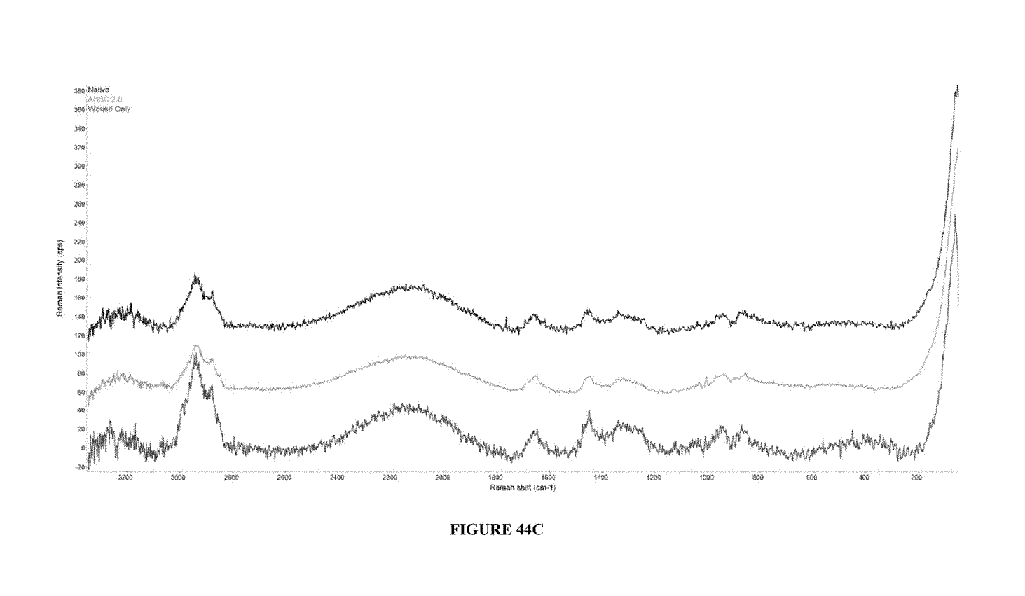

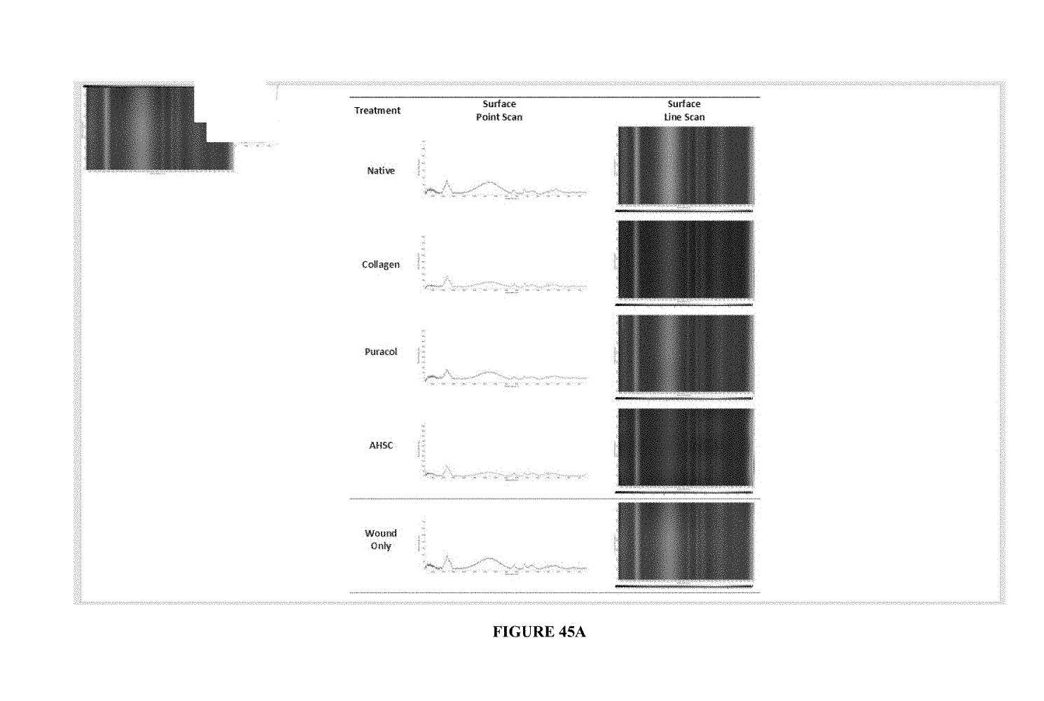

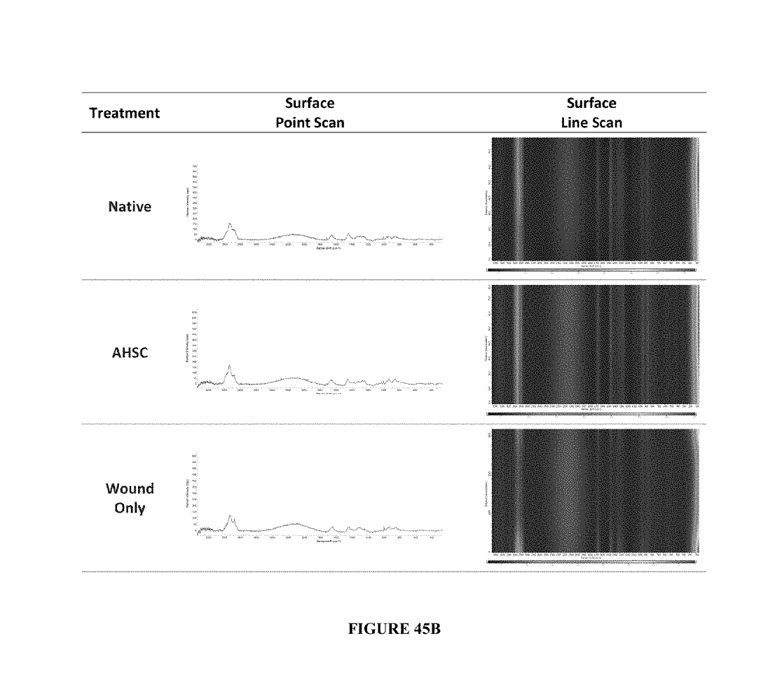

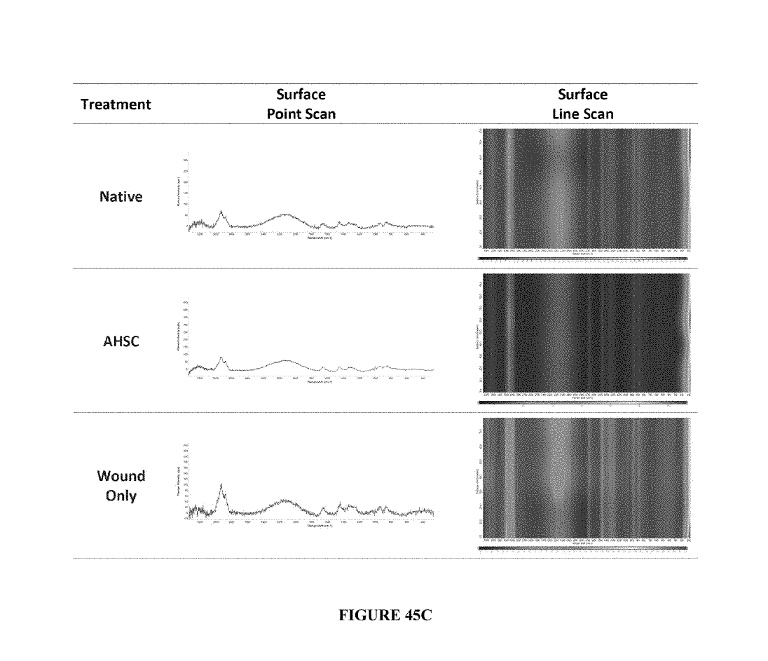

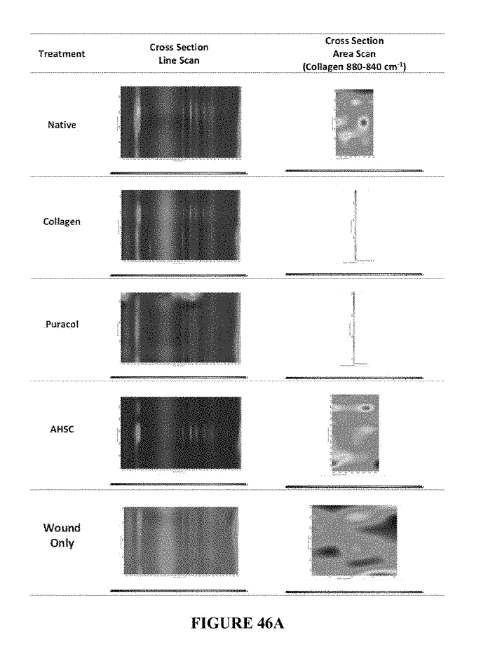

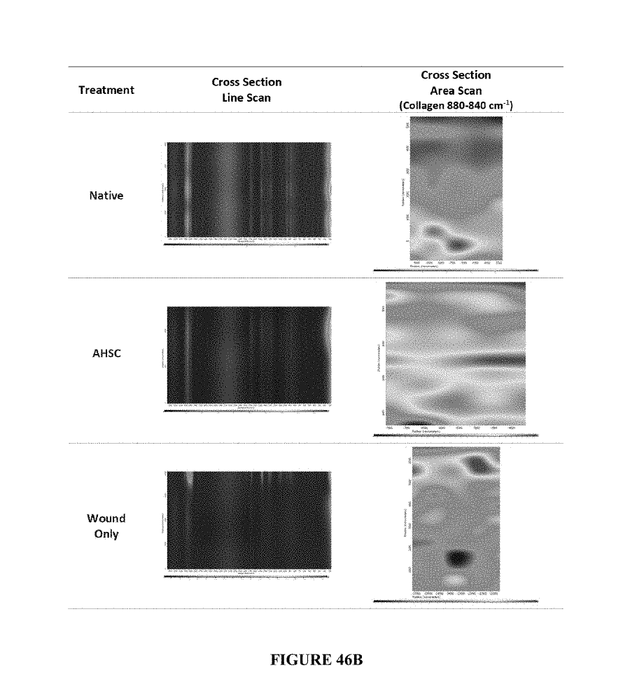

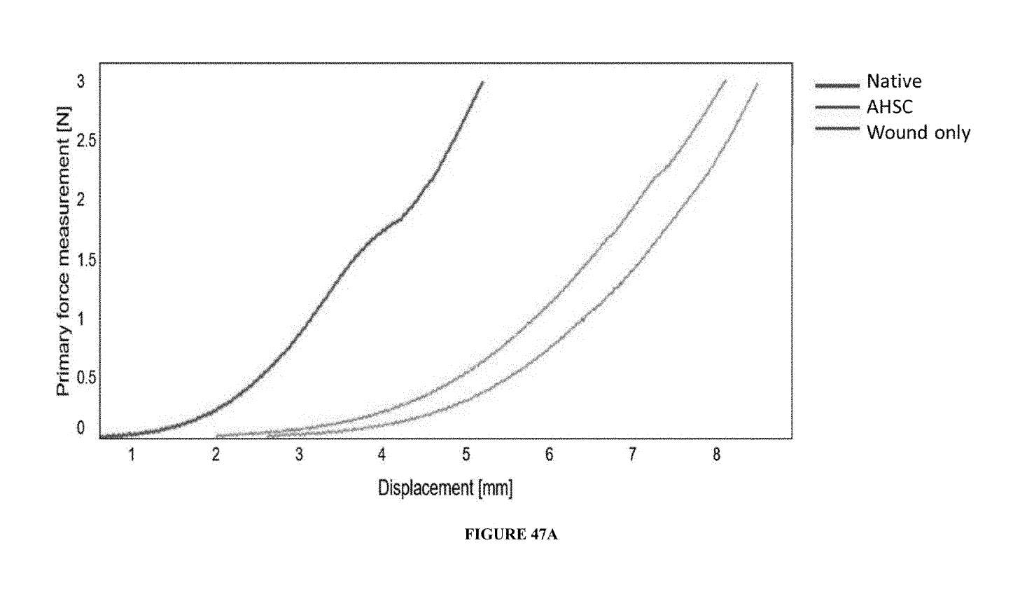

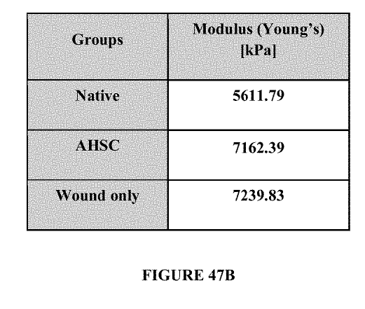

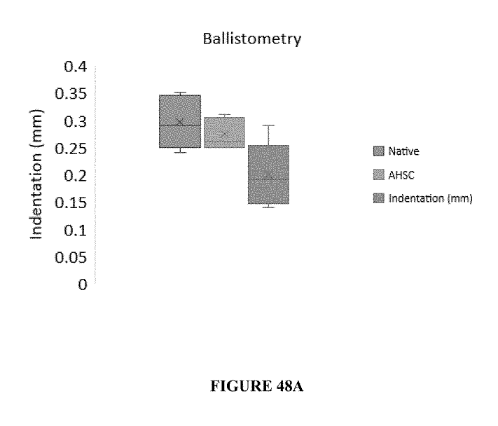

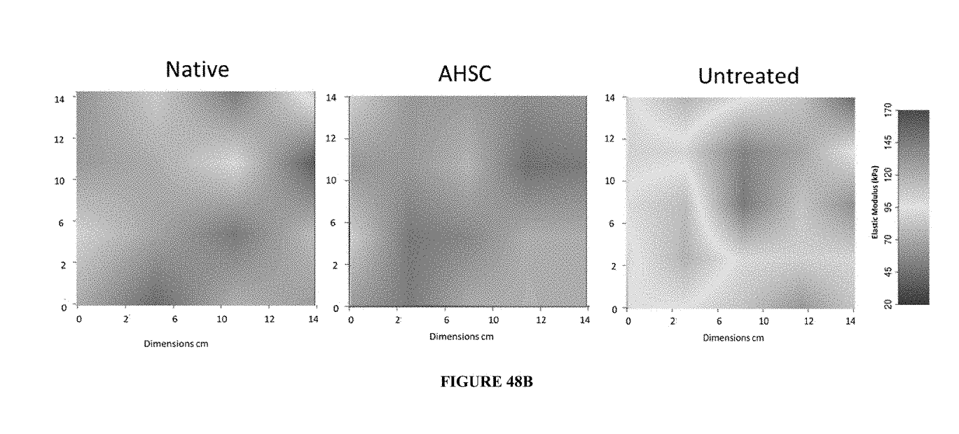

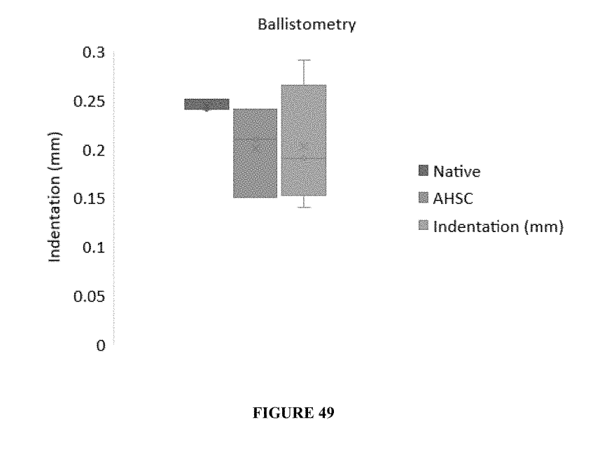

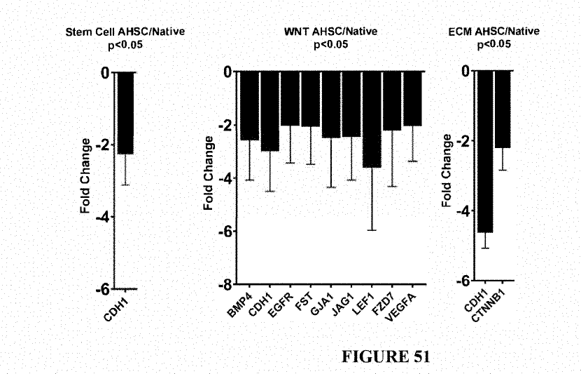

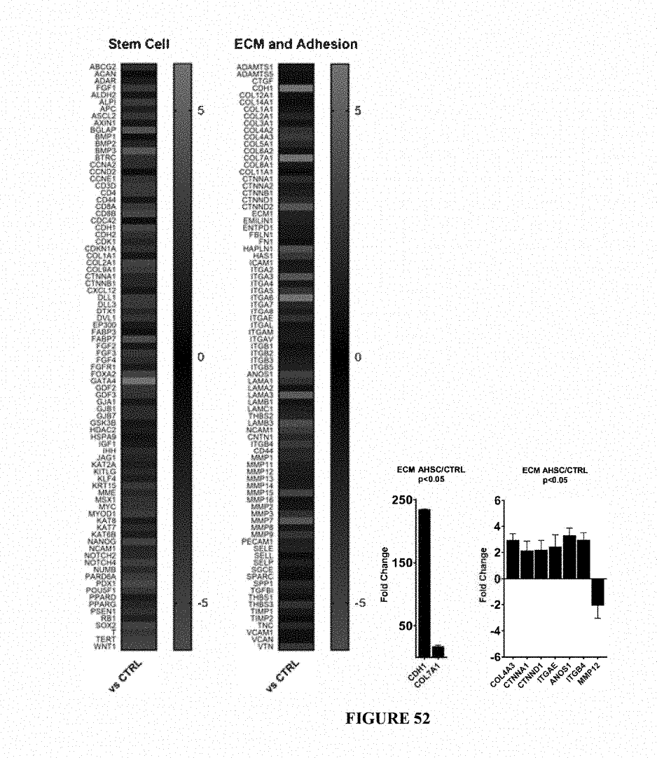

FIGS. 41-52 show the results of the study. Native skin and controls demonstrated functional skin characteristics as shown by minimal wound contraction, dermal and epidermal growth, and the presence of hair, glands, and vasculature as expected. Wound only (non-treated), collagen treated and Puracol treated controls demonstrated scarring, wound contraction, and minimal development of functional skin components including glands, hair follicles, and capillaries. The use of AHSC resulted in reduced wound contraction, new dermal and epidermal growth, new hair growth, and the presence of vasculature compared to non-treated wound only controls, collagen only or Puracol only treated wounds. The amount of a cutaneous-derived composition used in the current study promoted regeneration of ultrastructural features indicative of fully functional skin.

Treated wounds and native skin were excised and imaged. Compound microscopy showed improved healing and reduced contraction in wounds treated with AHSC. Histological staining with Masson's Trichrome, SEM, and multiphoton imaging demonstrated an organized extracellular matrix (ECM) indicative of full thickness skin. Confocal fluorescent microscopy revealed the presence of hair follicles, vasculature, and rete pegs at the epidermal-dermal interface highlighting the regeneration of functional skin.

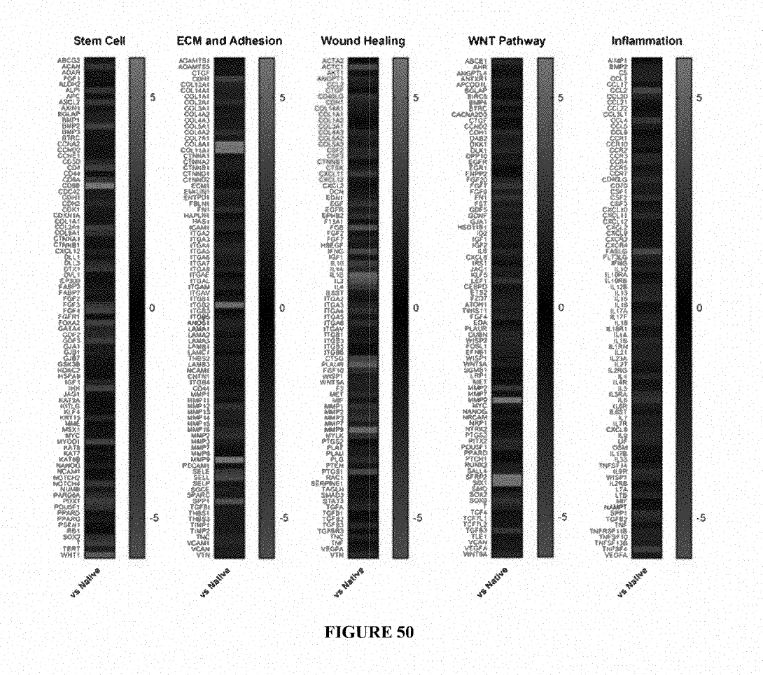

FIGS. 50-52 depict the tissue molecular analysis results of the study. Overall, gene expression changes are minimal when cutaneous-derived composition treated wounds are compared to native skin tissue. Significant down-regulation is observed for: CDH1, CTNNB1, BMP4, EGFR, FST, GJA1, JAG1, LEF1, FZD7, and VEGFA. All of these genes are known to have some role in or are targets of the WNT signaling pathway. Overall, trends are not noticeable for Stem Cell, E C M and Adhesion, and WNT Pathway profiles. Wound healing markers are overall upregulated and most inflammation markers tested are downregulated.

Minimal differential gene expression between cutaneous-derived composition treated wounds and native skin tissue suggests that cutaneous-derived composition treated wounds are almost indistinguishable from native skin at the molecular level. Significant changes in WNT Pathway players suggests that WNT signaling may be a critical mechanism by which wound healing is mediated in cutaneous-derived composition treatments.

The critical epithelial adhesion transcripts, CDH1 and COL7A1, are present in cutaneous-derived composition treated wounds and are absent in control wounds. CDH1 is necessary for cell-cell adhesion of epithelial cells, and COL7A1 has critical function as part of the basement membrane.

The expansion of the epidermis and growth of neodermal islands within the wound bed suggest this type of growth would continue until the wound is entirely repaired. The amount of a cutaneous-derived composition used in the current study promoted regeneration of ultrastructural features indicative of fully functional skin.

1. A composition comprising a stimulated heterogeneous mammalian tissue interface cell aggregate that is capable of producing functional polarized tissue when administered to a subject in need thereof.

2. The composition of claim 1, wherein the stimulated heterogeneous mammalian tissue interface cell aggregate is derived from an osseous tissue interface.

3. The composition of claim 2, wherein the osseous tissue interface is a peri-cortical tissue interface, a peri-lamellar tissue interface, a peri-trabecular tissue interface, a cortico-cancellous tissue interface, or any combination thereof.

4. The composition of claim 2, wherein the stimulated heterogeneous mammalian tissue interface cell aggregate comprises living core potent cellular entities and supportive entities.

5. The composition of claim 4, wherein the living core potent cellular entities express RNA transcripts and/or polypeptides of one or more Leucine Rich Repeat Containing G Protein-Coupled Receptors selected from the group consisting of LGR4, LGR5, LGR6, and any combination thereof.

6. The composition of claim 4, wherein the living core potent cellular entities express RNA transcripts and/or polypeptides of one or more of Pax 7, Pax 3, MyoD, Myf 5, or any combination thereof.

7. The composition of claim 2, wherein the stimulated heterogeneous mammalian tissue interface cell aggregate exhibits increased expression levels of parathyroid hormone, TLR4, and/or thymidine phosphorylase compared to that observed in native osseous tissue.

8. The composition of claim 2, wherein the functional polarized tissue shows decreased expression levels of one or more of IL2, MYOSIN2, ITGB5, and STAT3 compared to that observed in native osseous tissue.

8. (canceled)

9. The composition of claim 4, wherein the supportive entities comprise cellular populations, extracellular matrix elements, or any combination thereof, and optionally wherein the supportive entities comprise mesenchymal derived cellular populations.

10. The composition of claim 9, wherein the extracellular matrix elements comprise one or more of hyaluronic acid, elastin, collagen, fibronectin, laminin, extracellular vesicles, enzymes, and glycoproteins.

11. The composition of claim 2, further comprising a delivery substrate, wherein the delivery substrate comprises a scaffold.

12. The composition of claim 2, wherein the stimulated heterogeneous mammalian tissue interface cell aggregate has a diameter of about 40 to about 250 μm.

13. A kit comprising the composition of claim 2 and instructions for use.

14. A method for promoting tissue regeneration in a subject in need thereof comprising administering to the subject an effective amount of the composition of claim 2.

15. A method for treating a subject in need of tissue repair comprising administering to the subject an effective amount of the composition of claim 2.

16. The method of claim 15, wherein the subject is suffering from a degenerative bone disease or a bone fracture or break.

17. A method for preparing the composition of claim 2 comprising isolating at least a portion of a mammalian material interface to obtain a heterogeneous mammalian tissue interface cell aggregate, wherein the mammalian material interface comprises heterogeneous mammalian tissue interface cells; and stimulating the heterogeneous mammalian tissue interface cells.

18. The method of claim 17, wherein stimulating comprises mechanical stimulation, chemical stimulation, enzymatic stimulation, energetic stimulation, electrical stimulation, biological stimulation, or any combination thereof.

19. The method of claim 18, wherein chemical or biological stimulation comprises at least one of chemokine receptor binding, paracrine receptor binding, cell membrane alteration, cytoskeletal alteration, alteration of physiological gradients, addition of small molecules or addition of nucleotides and ribonucleotides.

20. A method for treating a subject in need of tissue repair comprising administering to the subject an effective amount of a composition comprising a stimulated heterogeneous mammalian tissue interface cell aggregate that is capable of producing functional polarized tissue when administered to a subject in need thereof, wherein administration of the composition results in an increase in at least one of parathyroid hormone, TLR4, thymidine phosphorylase in the subject compared to that observed prior to administration.
